(19) United States
(12) Patent Application Publication
(10) Pub. No.: US 2026/0234536 A1
(43) Pub. Date: Aug. 13, 2026

(54) POLYBIOSENSING AND IMAGING PLATFORM SYSTEM, METHOD AND DEVICE

(71) Applicant: POLYBIOMICS, INC., KIRKLAND, WA (US)

(72) Inventors: Mandana Veiseh, Kirkland, WA (US); David Stumbo, Boulder, CO (US); Bahram Bahrami, Kirkland, WA (US); Ava Aslanpour, Sunnyvale, CA (US); Shahab Sheikh Bahaei, Atherton, CA (US)

(21) Appl. No.: 19/508,259

(22) PCT Filed: Apr. 14, 2023
(86) PCT No.: PCT/US2023/018694

§ 371 Date: Feb. 3, 2026

Related U.S. Application Data
(60) Provisional application No. 63/331,510, filed on Apr. 15, 2022.
(60) Provisional application No. 63/334,001, filed on Apr. 22, 2022.
(60) Provisional application No. 63/334,009, filed on Apr. 22, 2022.

(51) Int. Cl. C12M 1/34; C12M 1/36; G01N 33/487

(52) U.S. Cl. CPC C12M 41/36; C12M 41/48; G01N 33/48735

(57) ABSTRACT

A polybiosensing-imaging system, method and device for continuous monitoring of multiple simultaneous parameters and functions of a living cell or cell clusters as contained in a well and/or controlled physiologically-relevant environment.

### RELATED APPLICATIONS

This application claims priority under 35 U.S.C. § 119(e) to the co-pending U.S. provisional patent application Ser. No. 63/331,510, filed Apr. 15, 2022, and entitled “PLATFORMS FOR PRECISE POLY BIOSENSING AND AUTOMATIC SELECTION OF IMAGE CAPTURE FREQUEN CY BY CONTINUOUS MOTION DETECTION,” co-pending U.S. provisional patent application Ser. No. 63/334,009, filed Apr. 22, 2022, and entitled “SINGLE CELL POLYSENSING AND IMAGING,” and co-pending U.S. provisional patent application Ser. No. 63/334,001, filed on Apr. 22, 2022, and entitled AHIGH THROUGHPUT POLYBIOSENSING AND IMAGE PLATFORM, @ all of which are hereby incorporated in its entirety by reference.

### FIELD OF THE INVENTION

The present invention is generally directed to the field of biosensors, biocompatible interfaces, bioinstruments for obtaining multiple cellular behaviors and environmental features. More specifically, the present invention is directed to systems and method of hosting, analyzing, manipulating and integrating information from biological samples using a polybiosensing-imaging system.

### BACKGROUND OF THE INVENTION

Technology is capable of sensing and tracking multiple physicochemical parameters and functions associated with the same cell(s) and other biological sample(s). Existing technologies require sensing of the various physicochemical parameters and functions of the related but different samples in separate environments and serially over time. Such restrictions may not be conducive for measuring changing characteristics over time or for deconvoluting the function of subpopulations that behave differently than the rest of the samples during processes such as cell progression from normal to disease state, or cell reaction over time to drugs, probes or toxins.

Characterization and control of biochemical processes may involve a multiplicity of intrinsic and extrinsic physical factors. Many processes are complex, and there may not be clear a-priori knowledge of what measurable factors may provide clear insight into the behavior of a given process. While there may be sensors available to measure individual physical or chemical factors, such as temperature, pH (acidity/alkalinity), or concentrations of substances in the reaction environment, introduction of said sensors can be disruptive to the process, and may be made difficult by the limited volume of some reaction spaces.

### SUMMARY OF THE INVENTION

Embodiments are directed to a polybiosensing-imaging system comprising a sensor positioning apparatus, an electrical interconnects substrate and well slide that includes one or more wells, such as the Genius WellTM by Polybiomics Inc., with integrated optical, chemical, electrochemical, and electromechanical sensors, imaging system, and software to capture and process multiple types of information from living biological samples. The test plates are able to function as multilayered transparent culture well(s) that hold(s) or transfer living sample(s) in native environment(s), and mechanically fit into a hybrid imaging and sensing system. The imaging system is able to view the evolving sample(s) and the sensors are able to measure and monitor multiple properties and biological functions (such as cellular growth, metabolism, movement, differentiation and transient events) according to user selected schedules and assays such as uptake of a functional imaging probe or drug. This approach may consider using multiple simultaneous phenotypic and functional data and/or information (i.e. PolyData™) that are captured from Genius Wells™ over time to improve optical imaging or image processing, including but not limited to electrical (such as impedance), biochemical, or optical modalities in order to improve sensitivity of detecting cellular metabolism, motility or tracking motion.

The polybiosensing-imaging system enables at least the following sensor positionings: sensors are connected to the well (sidewall or bottom); sensors are inserted inside the lid (e.g. Genius Wells™ lid), the wells (hanging); sensors are attached to the sidewalls (optical fibers); sensors are embedded in the wells (sidewall or bottom) and an imager performs multiple measurements of chemical analytes. Envisioned system deals with sensor cross-talks besides noise related to, for example, intrinsic biological noise, electrode noise, changed threshold due to continuous monitoring, or optical noise caused by vibration due to mechanical movements. The wells reside inside a cell culture incubator in an environmentally controlled condition (as far as temperature, humidity, CO2/O2 level) and/or an environmentally controlled condition is able to be structured around the wells and samples. One integrated system is able to perform simultaneous imaging (continuous from seconds to days), metabolite measurements (pH, Oxygen, and Glucose), and impedance measurements of live cells without perturbation of live cells.

Data is able to be captured, connected and processed for identifying noise or sensor cross-talks as part of cell analysis. Data from different modalities is able to be merged into one data set and time aligned, providing unified data at a given point in time. Synchronous data is able to be resampled and decimated at 3 different sampling rates: 10, 100, and 1000 seconds.

Information about which sensor is in which well is able to be looked up in experimental conditions database and translated. Signal unifications are able to continue until calibrated unified data, filtered unified data, and normalized unified data are generated according to a defined workflow.

Image analysis is able to run in the cloud and/or local memory (e.g. hard drive) and generate data structures representing living cells in time and space. These data structures are able to be stored in a database and their collection from an assay generate pairwise comparisons and correlations. By eliminating the biological noise inherent in comparing different measurements in different runs, repeatability issues are moved from biology to engineering. When all the measurements are performed on the exact same sample in the same experiment, the biological noise due to heterogeneity is minimized. Because in theory electrical signals can respond to, micro-/nano-environmental remodeling, such changes can be monitored sensitively and quantitatively too. In the polysensing and imaging system live samples are able to be preserved and tested in a physiologically relevant environment that mimic in-vivo condition. Some examples of the physiologically relevant environment are testing the samples in three-dimensional model, creating controlled environment as far as humidity, temperature, CO2 level, Oxygen level, pH, pressure, dissolved ions, glucose, polarity, viscosity, gas and liquid flow, and nutrition ingredient. These conditions can change to mimic disease model. For example, to mimic tumor microenvironment, live biological sample will be maintained and screened for any behavior or drug response analysis in high pressure, low oxygen level (hypoxic) or low pH (wherein the drugs are able to include, but are not limited to cellular therapeutics, where patient's live cells are used as therapeutic agents).

A first aspect is directed to a hybrid polybiosensing-imaging system. The system comprises a sensor positioning apparatus comprising a base having a well recess and a plurality of imaging holes within the well recess and a slide holder having a central slide window and one or more optical passages that extend from the central slide window to a perimeter of the slide holder opposite the central slide window and a well slide including a plurality of wells, the well slide positioned within the well recess such that each of the wells is above one of the imaging holes, wherein the slide holder is able to pivot with respect to the base between a closed position where the slide holder is substantially parallel to the base and the well slide is within the central slide window and an open position where the slide holder is angled away from the base.

In some embodiments, the system further comprises a lid covering each of the wells and including a separate sensor hole and syringe hole corresponding to each of the wells. In some embodiments, the lid further comprises a separate sensor support tab corresponding to each of the wells, wherein for each of the wells, the sensor support tab protrudes upward from the lid adjacent to a top opening of the sensor hole corresponding to the well. In some embodiments, the lid further comprises a separate rim guide corresponding to each of the wells, wherein for each of the wells, the rim guide protrudes downward from a bottom of the lid such that the rim guide abuts a top rim of the well. In some embodiments, for each of the wells, the sensor hole corresponding to the well is positioned adjacent an outer wall of the well and the syringe hole corresponding to the well is positioned adjacent to an inner wall of the well opposite the outer wall. In some embodiments, for each of the wells, a central axis of the syringe hole corresponding to the well is angled such that the central axis points from a top right corner of the inner wall of the well to a bottom left corner of the inner wall of the well. In some embodiments, the optical passages become narrower such that an outer opening of each of the optical passages facing the perimeter of the slide holder is larger than an inner opening of each of the optical passages facing the central slide window.

In some embodiments, the system further comprises one or more optical probes that each fit within the outer opening of one of the optical passages and having one or more optical fibers that fit within the inner opening of the one of the optical passages. In some embodiments, the system further comprises one or more guide plates coupled to the slide holder such that the guide plates extend over the central slide window, wherein when the slide holder is pivoted from the open position to the closed position, the guide plates contact angled outside-facing walls of the wells of the well slide causing the well slide to align with a center of the central slide window. In some embodiments, the system further comprises a flexible forked circuit including a network interface at a base of the circuit, one or more first electrical couplers at a first finger of the forked circuit and one or more second electrical couplers at a second finger of the forked circuit, wherein the first and second electrical couplers are configured to electrically couple the network interface with one or more sensors positioned within the sensor holes of the lid. In some embodiments, the system further comprises an electrical interconnect substrate including a plurality of electrodes positioned under the wells of the slide and an impedance printed circuit board configured to electrically couple with the electrodes when the slide holder is in the closed position. In some embodiments, the impedance printed circuit board is positioned on a printed circuit board platform at an end of the slide holder and has one or more electrical coupling pins that protrude through an impedance window of the end of the slide holder to electrically couple with the electrodes when the slide holder is in the closed position.

A second aspect is directed to a hybrid polybiosensing-imaging system. The system comprises a slide, a well wall including a holder configured to hold the slide and an optical assembly that mates to the outside of the well wall. In some embodiments, the system further comprises a chemical sensor element, wherein the slide is positioned between the chemical sensor element and the well wall. In some embodiments, the system further comprises a chemical sensor element positioned between the slide and the well wall. In some embodiments, the system further comprises a chemical sensor element positioned between the slide and the well wall and a reflective layer on a side of the slide opposite the chemical sensor element. In some embodiments, the system further comprises a chemical sensor element and a reflective layer, wherein the chemical sensor element is positioned between the reflective layer and the slide, and wherein the slide is positioned between the chemical sensor element and the well wall. In some embodiments, the optical assembly comprises a single optical fiber. In some embodiments, the optical assembly comprises a plurality of optical fibers.

In some embodiments, a first optical fiber of the plurality of optical fibers is configured for providing a stimulus to a chemical sensor, and additional optical fibers of the plurality of optical fibers are configured for performing calibration. In some embodiments, the optical assembly comprises a plurality of parallel optical fibers configured in a semi-circular phase. In some embodiments, the optical assembly further comprises a lens. In some embodiments, the system further comprises a multi-sensor chemical sensor element. In some embodiments, the optical assembly further comprises retractable pins configured to mate the optical assembly to the well wall. In some embodiments, the slide comprises notches configured for receiving pins of the well wall. In some embodiments, the system further comprises a clamp configured to mate the optical assembly to the well wall. In some embodiments, the clamp is angled to fit on the well wall. In some embodiments, the holder configured for holding the slide is configured for holding the optical assembly.

A third aspect is directed to a device. The device comprises a well structure, a sensor and a moveable wall within the well structure, the moveable wall configured to reduce a volume of media that is proximate to a cell and the sensor. In some embodiments, the sensor comprises an optical assembly. In some embodiments, the moveable wall comprises a piezo driver and a shaft. In some embodiments, the moveable wall comprises a flexible material. In some embodiments, the moveable wall comprises a rigid structure and a flexible structure. In some embodiments, the device further comprises a plurality of constricting walls. In some embodiments, each constricting wall of the plurality of constricting walls comprises a fluidic channel. In some embodiments, the device further comprises an electrode coupled to the moveable wall. In some embodiments, the device further comprises an imaging unit configured for acquiring an image of the cell. In some embodiments, the device further comprises a mechanism for performing depth calibration for the imaging unit. In some embodiments, the mechanism comprises equally spaced calibration spheres. In some embodiments, the mechanism comprises a spiral configuration.

A fourth aspect is directed to a hybrid polybiosensing-imaging system. The system comprises a well slide having two or more rows of a plurality of wells, a lid covering a top of each of the wells, a plurality of optical sensors that extend through the lid and into the wells and a light guide structure including a plurality of light guiding mechanisms exposed and extending from a bottom of the light guide structure through the light guide structure to a perimeter of the light guide structure, wherein the light guide structure is positioned on the well slide such that the light guide structure straddles two of the rows of wells and a top of each of the optical sensors under the light guide structure is aligned with one of the light guiding mechanisms exposed on the bottom of the light guide structure. In some embodiments, the optical sensors are positioned adjacent to sides of the wells. In some embodiments, each of the sensors has a chemical sensing dot that is coupled to a bottom of the sensors, the chemical sensing dot configured to fluoresce based on being exposed to a target chemical upon receiving excitation light.

In some embodiments, each of the wells has a chemical sensing dot is coupled to a top surface of a floor of the well, the chemical sensing dot configured to fluoresce based on being exposed to a target chemical upon receiving excitation light. In some embodiments, the system further comprises a plurality of insert sheets each positioned on a floor of one of the wells and having a chemical sensing dot coupled to a top surface of one of the insert sheets, the chemical sensing dot configured to fluoresce based on being exposed to a target chemical upon receiving excitation light. In some embodiments, each of the insert sheets has a central hole. In some embodiments, the system further comprises a plurality of alignment structures each positioned on the lid above the top of one of the optical sensors, each of the alignment structures having a central channel that is aligned with the one of the optical sensors and tapered outer side walls. In some embodiments, a bottom of the light guide structure has recesses surrounding a bottom of each of the light guiding mechanisms, wherein tapered inner walls of each of the recesses are congruent with the tapered outer side walls. In some embodiments, one or more of the light guiding mechanisms comprise a plurality of optical fibers in optical communication with each other via one or more mirrors.

In some embodiments, the system further comprises one or more additional light guiding mechanism positioned within one or more of the floor and side walls of the wells. In some embodiments, the system further comprises a plurality of lid protrusions that each protrude downward from the lid into one of the wells. In some embodiments, the lid protrusions include a plurality of apertures whose opening sizes are each based on a vertical position of the aperture on the lid protrusion. In some embodiments, each of the lid protrusions include a first electrode and each of the wells includes a second electrode. In some embodiments, the system further comprises a light guiding mechanism interrogator that selectively rotates one or more optical fibers such that the fibers align with a subset of tops of the light guide mechanisms.

A fifth aspect is directed to a system. The system comprises a well sealed against oxygen permeation, a fluorescent sensor within the well, the fluorescent sensor configured for measuring analyte concentration, a metering device configured for providing oxygen to the well and a measuring device configured to measure an oxygen level to control the metering device and keep the measured oxygen level constant. In some embodiments, the system further comprises a gain block configured to receive analyte concentration information and analyte target concentration information. In some embodiments, the gain block is further configured to generate an analyte metering control signal. In some embodiments, the analyte concentration information is received from the measuring device. In some embodiments, the metering device comprises a pump. In some embodiments, the measuring device comprises a fiber.

### BRIEF DESCRIPTION OF THE DRAWINGS

FIG. 1 illustrates a conceptual diagram of a polybiosensing-imaging system in an exemplary implementation according to some embodiments.

FIG. 2 illustrates a perspective view of an electrical interconnects substrate according to some embodiments.

FIG. 3 illustrates a perspective view of a well slide attached to the electrical interconnects substrate in FIG. 2 according to some embodiments.

FIG. 4 illustrates a cut-out side view of an exemplary well having a biological sample to be sensed according to some embodiments.

FIG. 5 illustrates a cut-out side view of an imaging unit according to some embodiments.

FIG. 6 illustrates a cut-out side view of an imaging unit according to some embodiments.

FIG. 7A illustrates a top perspective view of the polybiosensing imaging system with the sensor positioning system in a closed position according to some embodiments.

FIG. 7B illustrates a top perspective view of the polybiosensing imaging system with the sensor positioning system in an open position according to some embodiments.

FIG. 7C illustrates a top perspective view of the sensor positioning assembly in a closed position according to some embodiments.

FIG. 7D illustrates a top perspective view of the sensor positioning assembly in an open position according to some embodiments.

FIG. 7E illustrates a perspective view of a guide plate having two prongs according to some embodiments.

FIG. 7F illustrates a perspective view of a guide plate having one prong according to some embodiments.

FIG. 7G illustrates a perspective view of a push rod according to some embodiments.

FIG. 7H illustrates a perspective exploded view of the hinge mechanism according to some embodiments.

FIG. 7I illustrates a top view of the base plate according to some embodiments.

FIG. 7J illustrates a top perspective view of the base plate according to some embodiments.

FIG. 7K illustrates a bottom perspective view of the base plate according to some embodiments.

FIG. 7L illustrates a top perspective view of the slide holder according to some embodiments.

FIG. 7M illustrates a bottom perspective view of the slide holder according to some embodiments.

FIG. 7N illustrates a top view of the slide holder according to some embodiments.

FIG. 7O illustrates a bottom view of the slide holder according to some embodiments.

FIG. 7P illustrates a side view of the slide holder according to some embodiments.

FIG. 7NN illustrates a top cross-sectional view of the slide holder at line NN of FIG. 7P according to some embodiments.

FIG. 7Q illustrates a top perspective view of the impedance PCB according to some embodiments.

FIG. 7R illustrates a perspective view of the sensor interconnect circuit according to some embodiments.

FIG. 7S illustrates a top view of a sensor according to some embodiments.

FIG. 7T illustrates a top perspective view of the lid according to some embodiments.

FIG. 7U illustrates a bottom perspective view of the lid according to some embodiments.

FIG. 7V illustrates a top view of the lid according to some embodiments.

FIG. 7W illustrates a front view of the lid according to some embodiments.

FIG. 7X illustrates a side view of the lid according to some embodiments.

FIG. 7Y illustrates a method of implementing the SPA according to some embodiments.

FIG. 8A illustrates a high-level view of the variant of well construction according to some embodiments.

FIG. 8B illustrates a combination of a slide, a well wall and an optical assembly according to some embodiments.

FIG. 8C illustrates a combination of a slide and a well wall according to some embodiments.

FIG. 8D illustrates an alternate construction of a slide and a well wall according to some embodiments.

FIG. 8E illustrates an alternate construction of a slide and a well wall according to some embodiments.

FIG. 8F illustrates an alternate construction of a slide and a well wall according to some embodiments.

FIG. 8G illustrates a front view of an optical assembly according to some embodiments.

FIG. 8H illustrates a front view of an optical assembly with multiple fibers according to some embodiments.

FIG. 8I illustrates a diagram of a composite assembly with parallel fibers and a semi-circular phase for higher effective NA collection according to some embodiments.

FIG. 8J illustrates a variant of an optical assembly according to some embodiments.

FIG. 8K illustrates a multi-sensor element according to some embodiments.

FIG. 8L illustrates a diagram of an optical assembly and a well side-wall using pin/protrusions according to some embodiments.

FIG. 8M illustrates a mechanism to align the slide/sensor element to the well side-wall using pins/protrusions provides lateral mechanical alignment according to some embodiments.

FIG. 8N illustrates a side view of well wall exterior according to some embodiments.

FIG. 8O illustrates a side view of a gap between the well wall and an optical assembly according to some embodiments.

FIG. 8P illustrates a side view of a diagram of a clip-on attachment according to some embodiments.

FIG. 8Q illustrates a side view of a diagram of a clip-on attachment according to some embodiments.

FIG. 9A illustrates a top view of an assembly with solid state sensors arranged on a plane according to some embodiments.

FIG. 9B illustrates a side view of an assembly with solid state sensors arranged on a plane according to some embodiments.

FIG. 9C illustrates a top view of an assembly with solid state sensors arranged in a stacked fashion according to some embodiments.

FIG. 9D illustrates a side view of an assembly with solid state sensors arranged in a stacked fashion according to some embodiments.

FIG. 9E illustrates a view of a generalized shape of an assembly with solid state sensors according to some embodiments.

FIG. 9F illustrates a diagram of an opening within a well wall according to some embodiments.

FIG. 9G illustrates a diagram of a sensor assembly within an opening within a well wall according to some embodiments.

FIG. 9H illustrates top views of a collapsible well according to some embodiments.

FIG. 9I illustrates top views of a collapsible well according to some embodiments.

FIG. 9J illustrates top views of a collapsible well according to some embodiments.

FIG. 9K illustrates top views of a collapsible well according to some embodiments.

FIG. 9L illustrates top views of a collapsible well according to some embodiments.

FIG. 9M illustrates top views of a collapsible well according to some embodiments.

FIG. 9N illustrates a side view of a mechanism for more accurate depth calibration for the imaging system according to some embodiments.

FIG. 9O illustrates a top view of a mechanism for more accurate depth calibration for the imaging system according to some embodiments.

FIG. 10A illustrates a perspective view of a polybiosensing-imaging system for high throughput according to some embodiments.

FIG. 10B illustrates a side cross-sectional view of a polybiosensing-imaging system for high throughput according to some embodiments.

FIG. 10C illustrates a top cross-sectional view of a polybiosensing-imaging system for high throughput according to some embodiments.

FIG. 10D illustrates a close up side cross-sectional view of the lid and one of the wells according to some embodiments.

FIG. 10E illustrates a close up side cross-sectional view of the lid and one of the wells with sensor-attached chemical sensor dots according to some embodiments.

FIG. 10F illustrates a close up side cross-sectional view of the lid and one of the wells with well-attached chemical sensor dots according to some embodiments.

FIG. 10G illustrates a close up side cross-sectional view of the lid and one of the wells including an insert having sensor dots according to some embodiments.

FIG. 10H illustrates a top view of an insert according to some embodiments.

FIG. 10I illustrates a side cross-sectional view of the lid, the support structure, one of the wells and alignment protrusions separated according to some embodiments.

FIG. 10J illustrates a side cross-sectional view of the lid, the support structure, one of the wells and alignment protrusions coupled together according to some embodiments.

FIG. 10K illustrates a close up side cross-sectional view of the lid, the support structure having discontinuous light guides, mirrors and one of the wells according to some embodiments.

FIG. 10L illustrates a close up side cross-sectional view of the wells including different well light guide structures according to some embodiments.

FIG. 10M illustrates alternate embodiments of the well light guide structures according to some embodiments.

FIG. 10N illustrates a side cross-sectional view of the lid and one of the wells with a lid protrusion according to some embodiments.

FIG. 10O illustrates a top view of a lid protrusion according to some embodiments.

FIG. 10P illustrates a bottom view of a lid protrusion according to some embodiments.

FIG. 10Q illustrates a method of implementing a high throughput polybiosensing-imaging system according to some embodiments.

FIG. 10R illustrates an interrogator according to some embodiments.

FIG. 11 illustrates a diagram of an implementation to accurately measure analyte consumption at controlled concentrations according to some embodiments.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed. Embodiments of the present application are directed to a polybiosensing-imaging system. Those of ordinary skill in the art will realize that the following detailed description of the polybiosensing-imaging system is illustrative only and is not intended to be in any way limiting. Other embodiments of the polybiosensing-imaging system will readily suggest themselves to such skilled persons having the benefit of this disclosure.

Reference will now be made in detail to implementations of the polybiosensing-imaging system as illustrated in the accompanying drawings. The same reference indicators will be used throughout the drawings and the following detailed description to refer to the same or like parts. In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application and business related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

A polybiosensing-imaging system is capable of tracking multiple phenotypical, functional and/or physicochemical parameters of biological samples non-invasively and continuously. The polybiosensing-imaging system and methods of using the polybiosensing-imaging system enable new ways of detecting, monitoring, and interrogating live cell for various application such as multiplexed biomarkers analysis. For example, new polymodal signatures and temporal/spatial correlations can be measured that would otherwise be missed by static endpoint measurements on dead cells, single endpoint measurements one live cells and/or merging of single mode signatures sensed by separate equipment or at different times. Intrinsic and/or extrinsic sample heterogeneity can be detected and predicted. Rates of heterogeneity and regulatory elements can be compared to explore pattern of variations in depth.

In some embodiments, the polybiosensing-imaging system includes wells having multiple different sensor types integrated into, attached onto, and/or inserted into each well, and an incubator-friendly reader for continuous and simultaneous capture of physicochemical, functional and/or phenotypic characteristics from biological samples. The polybiosensing-imaging system enables hosting, analyzing, and manipulating biological samples within each well. In other embodiments, every well does different sensing but at the same time.

Some examples for modalities of measurements include, but are not limited to, electrical, optical, acoustic and chemical including ion measurements. For example, the system is able to perform: visible light imaging of the samples/cells via an imaging unit of an illumination and microscopy platform; fluorescent light imaging of the samples/cells via the imaging unit; monitoring of local temperature pressure, humidity and carbon dioxide (CO2) (e.g. via incubator sensors, lid sensors and/or well sensors); monitoring of ambient oxygen (O2) levels and/or O2 levels in the media and/or samples within the wells; monitoring of pH conditions in the media and/or samples within the wells; monitoring of ambient CO2 levels and/or CO2 levels in the media and/or samples within the wells; and monitoring of lactate, glucose and/or other chemistries of interest levels in the media and/or samples within the wells.

Some of the sensors are thin Piezoelectric transducers (PZT) or ultrasonic transducers on the sidewalls of the well or on the inserts that are placed inside the well. Some of sensors are a large array of densely packed and biocompatible PZTs closer to cell clusters. Some are miniaturized sensitive PZT sensors that can distinguish between signal attenuation and random phase change or destructive or constructive interferences, because different locations of the cell clusters may receive different amounts of sound waves. Those include sparse array of transducers at sizes that are one-half of the wavelength. In all cases, a mechanism to provide multiplexing of isolated wires to fewer number of wires in a defined spatial configuration according to form factor of the well or inserts are considered. Some examples of chemical sensors are sensor dots and foils that measure chemical analytes and create signals such as optical or electrical when exposed to different analytes.

Examples of sensors include, but are not limited to, ion-sensitive FETs, chemical-sensitive FETs or sensors whose color changes, sensors whose electrical or chemical, or physical properties change. Ion concentrations can be measured in various ways such as ion-selective membrane based sensors. When this membrane is immersed in the fluid under test, a potential is generated that scales with the logarithm of the ion activity under test, which is a measure for its concentration. Such ion selective electrode can be significantly miniaturized. An example is the chemical field effect transistor (FET) in which the modified gate of a FET is in contact with the fluid under test and influences the source-drain current depending on the ion level of interest.

Any ion selective sensor consists of two essential parts: the ion selective electrode is the first, while the second is the reference electrode: the ion selective electrode is immersed in the solution under test as well and the reference electrode potential should be independent of the solution composition. One example of a commonly used type of reference electrode is a silver chloride (AgCl) electrode in contact with a reservoir with a fixed Cl-concentration. The internal reservoir is separated from the fluid under test by a porous frit (junction). The reference potential remains stable if the Cl-concentration remains unaltered. Some examples of a miniaturized, long-term stable reference electrode planar AgCl for Cl-measurements or Iron oxide for pH measurements on a Si substrate to form the multi-ion sensor. Other types of sensors are immersion sensors that are connected to a chemical reader through wired or wireless connections and measure chemical analyte changes inside the well. Another example is a system that collects samples from each well and send the samples to a chemical reader to sense and analyze chemical changes. Chemical sensors also can be implemented as dyes that can be sensed using a fluorescence process.

In general, sensors can be implemented either as probes inserted into the well, they can be implemented as packets that are dropped in the well, they can be printed on the bottom or side of the well surface, and/or they can be attached to the bottom or side of the well surface. The dyes could be fluorescent voltage-sensitive dyes to detect mitochondrial function as a measure of cell energetic activities. A distinctive feature of the early stages of apoptosis is the disruption of the mitochondria, including changes in membrane and redox potential, which can be tracked specifically by assaying mitochondrial membrane potential using dyes that are positively charged. Dyes accumulate in the electronegative interior of the mitochondrion.

When extending this concept to a multi-well implementation, the different wells can either have identical sensor capabilities, or different wells can be equipped with different sensor types (e.g. electrochemical and/or enzyme-based glucose or lactate sensors, or other types of sensors described herein). In other words, mix and match of different sensor types for different wells can be applied. Optics or electronics are used to measure the sensor outputs of the different wells.

A biological sample may be obtained from a subject. A subject can be a biological entity containing expressed genetic materials. The biological entity can be single or multiple cells, cell compartment, tissue, organel, organoid, plant, animal, or microbe, including, e.g., bacteria, bacterial plasmids, viruses, fungi, and protozoa. The subject can be tissues, single cells, cell clusters and their progeny of a biological entity obtained in vivo or cultured in vitro. The subject can be a mammal. The mammal can be a human. A biological sample may be an environmental sample. Examples of environmental samples can include air, water, soil, agricultural, or geologic.

A biological sample can comprise a plurality of cells. A plurality of cells may be present in a variety of three dimensional structures. A plurality of cells can be adherent, suspended, cultured on a substrate such as extracellular matrices, gel, hydrogel, or a combination thereof. A plurality of cells can be adherent to one another or to a surface. Cells can be adherent to a surface and present in a monolayer, bi-layer, multilayer, 3D structure, organic spheroid and the like. A plurality of cells can be heterogeneous or homogenous. A plurality of cells may be initially homogenous and change over time to become heterogeneous. A plurality of cells may be heterogeneous and the heterogeneity may change over time, along with the properties of the cells. An example of a heterogeneous cell population that may change over time are cancer cells, which may exhibit abnormal proliferation or differentiation (e.g., as exhibited in tumor growth or tumor metastasis).

A biological sample may be cell, cell compartment, tissue, organelle, organoid, solid matter, such as biological tissue. A biological tissue may comprise a plurality of cells, such as primary cells, cell lines, suspension cells, stem cells, progenitor cells from different type and tissue such as endothelial cells, fibroblasts, stellate cells, and the like.

A biological sample may be a fluid, such as biological fluid such as blood or cells in a culture media. A biological fluid can include any fluid associated with living organisms. A biological fluid may include components within the fluid. For example, a biological sample can include blood with components of the blood, such as white blood cells, red blood cells, platelets, and the like, and components thereof. A biological sample may comprise cellular components, including, for example, biomolecules and intracellular structures. Non-limiting examples of biomolecules include proteins, nucleic acids, lipids, carbohydrates, hormones, extracellular matrix, extracellular components, secretome, or exosomes, and the like. Non-limiting examples of intracellular structures include organelles such as vesicles, mitochondria, lysosomes, centrosomes, exosomes, etc. A biological sample may comprise in vitro models, such as induced pluripotent stem cells (iPS), spheroids, organoids, in vitro fertilization samples (e.g., eggs, sperms, embryo), or tumor models. A biological sample, such as tissue, may be cultured in a three-dimensional environment. A biological sample may comprise non-host components, such as bacteria, viruses, fungi, yeast, nematodes, or other microbes.

A biological sample may be analyzed to detect a single analyte (e.g., protein, amino acid, or nucleic acid) or multiple analytes (e.g., protein and nucleic acid). The multiple analytes may be detected concurrently or subsequently. Analytes may be cellular and/or acellular analytes.

Non-limiting examples of cellular analytes may include ions, proton, oxygen, peptide, protein, enzymes, exosomes, or nucleic acid molecules.

Analytes of a biological sample may be detected by labeling the analytes. Analytes may be coupled to a label for detection by a sensor. A label may be a composition that yields a detectable signal, indicative of the presence or absence of the analyte (e.g. chemical sensor dot). A label may be directly detectable label (e.g., a fluorescent label). A fluorescent label may be any fluorescent label such as a fluorescent label (e.g., fluorescein, Texas red, rhodamine, ALEXAFLUOR7 labels, and the like), a fluorescent protein (e.g., GFP, EGFP, YFP, RFP, CFP, cherry, tomato tangerine, and any fluorescent derivate thereof). A label may be indirectly detectable label (e.g., a binding pair member). An indirect label may include biotin (a binding pair member), which may be bound by streptavidin (which may itself be directly or indirectly labeled). Non-limiting examples of labels include: a radiolabel (a direct label) (e.g., 3H, 125I, 35S, 14C, or 32P); an enzyme (an indirect label) (e.g., peroxidase, alkaline phosphatase, galactosidase, luciferase, glucose oxidase, and the like); a fluorescent protein (a direct label) (e.g., GFP, RFP, YFP, and any derivatives thereof); a metal label (direct label); a colorimetric label; a binding pair member; nanoparticles such as metalioc, non-metalic, or polymetic based and the like. Binding pair member may refer to one of a first and a second moiety, wherein the first and the second moiety have a specific binding affinity for each other. Non-limiting examples of binding pairs include: antigen/antibody (e.g., digoxigenin/anti-digoxigenin, dinitrophenyl (DNP)/anti-DNP, dansyl-X-anti-dansyl, fluorescein/anti-fluorescein, Lucifer yellow/anti-lucifer yellow, and rhodamine anti-rhodamine), biotin/avidin (or biotin/streptavidin) and calmodulin binding protein (CBP)/calmodulin. Any binding pair member may be suitable for use as an indirectly detectable label.

A label may be detected using suitable detection methods. For example, cellular components such as mitochondria may be labeled with a directly detectable label, such as a fluorescent label (e.g., MitoSox Red dye). The fluorescent label may be detected using an optical measuring modality. In another example, cellular components such as proteins may be detected using a binding member pair, such as antigen/antibody. The protein may be contacted with a labeled primary or labeled secondary antibody and binding of the protein with the labeled antibody may be detected using suitable modality, such as chemical modality. The chemical modality may detect activity of an enzyme (e.g., peroxidase) coupled to the antibody, indicative of binding of the antibody to the protein.

Analytes may be detected using label-free techniques. Label-free detection may be accomplished, for example, using label-free imaging, sensor dots, electrical, impedance, spectrometric methods, magnetic, microscopy, biomolecular interactions, chemical, electrochemical, electromechanical, or acoustic measurements.

Analytes may be cellular components, such as nucleic acid molecules, DNA or RNA, for example. Nucleic acid molecules may be coupled to a label for detecting the nucleic acid molecules. Nucleic acid molecules may be processed prior to detection. For example, nucleic acid molecules may be amplified, prior to detection. In such cases, the label may be detectable as nucleic acid molecules undergo amplification. In another example, nucleic acid molecules, such as RNA, may be reverse transcribed in order to detect the nucleic acid molecules. Labels may be covalently or non-covalently (e.g., ionic interactions) coupled with the nucleic acid molecules. In some cases, a label coupled to nucleic acid molecule may be an optically-active dye (e.g., a fluorescent dye). In some cases, a label may be a sequence-specific oligonucleotide probe that is optically active when hybridized with a complementary nucleic acid molecule. In some other cases, a label may be a radioactive species. Methods for detecting nucleic acid molecules may include optical detection methods (e.g., fluorimetry and UV-vis light absorbance), spectroscopic detection methods (e.g., mass spectrometry, nuclear magnetic resonance (NMR) spectroscopy, and infrared spectroscopy), electrostatic detection methods (e.g., gel based techniques, such as, gel electrophoresis) or electrochemical detection methods (e.g., electrochemical detection of amplified product after high-performance liquid chromatography separation of the amplified products).

Modalities may be selected based on the detection methods. For example, optical measuring modality may use a confocal microscopy module for detecting a fluorescent label. In another example, an impedance measuring modality may use a module for measuring conductivity in order to determine the changes in impedance. Chemical measuring modality may use detect products of a chemical reaction, selective ion, polarity, such as substrate conversion by an enzyme. Acoustic measuring modality may detect absorbance and/or transmission of sound waves through a biological sample for measuring stiffness.

Signals obtained from the detection method using one or more modalities may be measured by one or more sensors routed to and from the wells. The measured signal may be combined or separately analyzed to generate a profile for a biological sample, such as a cell population. For example, the signals from an optical measuring modality may be measured by sensors to determine physical, and/or chemical profile of the cell population. The signals may be used to determine physical profile, such as morphological profile including size, shape, cellular components, and the like. The signals may be used determine chemical profile, such as concentration of ion, peptide, amino acid, protein, antibody, carbohydrate, lipid, biomolecule, DNA, RNA, exoxome analytes.

Each individual well is formed for storing a fluid sample. A fluid sample is a gel, liquid, or other medium that includes the biological sample to be analyzed. Although subsequently described below in terms of analyzing a cell, it is understood that application is not limited to a cell, or a cell colony, and generally applies to a biological sample. Each well can also contain a culture media that is specific for each biological sample to be analyzed and provides, for example, nutrient materials, serum, and/or antibiotic for culturing each sample type or cell flowing in a media or liquid that is passing through polysensors.

The polybiosensing-imaging system enables at least the following features: sense sample within the environment of the well; extract sample from the environment of the well and sense outside of the well; extract sample from the environment of the well and sense on microfluidics incorporated wells; a mechanism of preventing penetration of cells (like a cut-off filter) to the microfluidics that sample some media from the cellular environment and sense chemicals of the media; a mechanism of sampling media from the cellular environment and sensing exosomes; a mechanism of sensing exosomes in the entire well; a mechanism to clip the well holder to the moving stage to improve quality of continuous imaging. The polybiosensing-imaging system also enables at least the following sensing features: sensors are connected to the well (sidewall or bottom); sensors are inserted inside the wells (hanging); sensors are attached to the sidewalls (optical fibers); sensors are embedded in the wells (sidewall or bottom) and an imager performs multiple measurements of chemical analytes; methods for optimizing noise and cross-talk.

The multiple sensors placed directly into the wells (side and/or bottom) or on inserts that go inside the wells generate signals from biological samples and analyte(s) continuously. The envisioned system, which is able to include a polysensor incorporated culture plate (PICP), deals with sensor cross-talks besides noise related to, for example, intrinsic biological noise, electrode noise, changed threshold due to continuous monitoring, or optical noise caused by vibration due to mechanical movements.

In some embodiments, the wells reside inside a cell culture incubator 2 in an environmentally controlled condition (as far as temperature, humidity, CO2/O2 level) and enables simultaneous imaging, metabolite measurements (pH, Oxygen), and impedance measurements of live cells using one integrated system continuously over hours to days without perturbation of live cells. Such polymodal multiplexing approach: a) eliminates rigorous signal to noise elevation and spectral subtractions processes that are associated with unimodal fluorescent multiplexing thus leads to higher precision, b) provides kinetics data through continuous measurements, c) minimizes labor and operation errors because one trained operator would run the system, d) provide multiple yet complementary information about live cells e) generates previously-unattainable cellular insight that enables informed decision making.

In particular, this approach can reveal new dynamic spatiotemporal correlations that would otherwise be missed by non-continuous measurements on fixed/dead cells or merging of single measurements made by separate instruments or at different times on live cells. The polybiosensing-imaging system also enables at least the following sampling features: sense sample within the well by inserting sensors into the well; extract fluid samples from the well and run through sensors that are external to the well; extract fluid samples from the well and run through sensors that are embedded in the well; extract fluid samples from the well and run through sensors that are embedded in the hanging inserts or as part of flow-through loop; methods for optimizing noise and cross-talk.

The approach used herein generates mass amounts of datasets without perturbing or changing the environment of the sample, which leads to better noise recognition and optimization. This ensures that a readout is not the result of under sampling and minute cell-intrinsic changes can be detected at higher probability. To optimize the placement of the sensors in multiple locations, profile spatial and temporal monitoring and measurement of O2 and pH can be profiled, taking into consideration the diffusivity of dissolved O2 (dO2) and ions such as H+ and OH− into cell culture nutrients according to Fick's Law. This profiling may help support use of a sensor in the wells described herein to provide information about the entire cell culture environment and subtle changes. Because cells make up a small volume of cell culture, changes in the concentration of oxygen in the media due to the cells will be minute and hard to distinguish from environmental noise.

In all cases, the cross-talk and noise is able to be quantified. If high cross-talk or occlusion of optical path inhibits optimal performance of the wells described herein for poly-biosensing and imaging, select sensors are able to be placed in different wells to accommodate fewer sensors per well, thus poly-biosensing and imaging can be performed per plate that holds multiple wells. In some cases, signal to noise can be managed by high localization of signal where the measurement is done at short wavelengths/high frequencies (in case of acoustic impedance sensing). In some cases, it is managed by having a-priori knowledge of where the cell is, so that one can focus the measurement to/about it. In other cases, averaging is done to sense a change in the cells out of a stable inert (non-changing) background.

### Polybiosensing-Imaging System

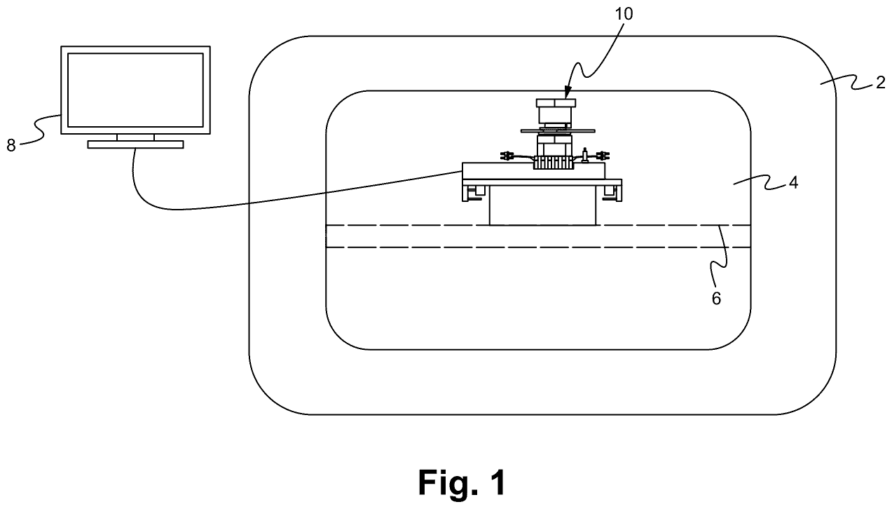

FIG. 1 illustrates a conceptual diagram of a polybiosensing-imaging system in an exemplary implementation according to some embodiments. A polybiosensing-imaging system 10 is configured to be removably installable within an interior chamber 4 of an incubator 2. In some embodiments, the polybiosensing-imaging system 10 is configured to be positioned on a rack 6 within the interior chamber 4. The polybiosensing-imaging system 10 is coupled to an external electronic device 8. In some embodiments, the incubator 2 includes an interface (not shown) that enables electrical and network interconnects to be made between devices within the incubator 2, such as the polybiosensing-imaging system 10, and devices external to the incubator 2, such as the external electronic device 8. In other embodiments, the incubator can be a smaller chamber that controls pressure, temperature, humidity, O2, and CO2 level around the cell (not shown), in other words a small incubator may only cover the area around the cell culture chamber and not an entire area around the imager and other components of the polybiosensing-imaging system. In some embodiments, relative humidity and temperature in the wells 24 is able to be detected (e.g. via immersed sensors 710 or lid 26 sensors). In some embodiments, metabolic activity in each of the wells 24 is able to be identified based on the well media/fluid temperature (and/or changes thereto). In some embodiments, the external electronic device 8 is one or more host processing devices, such as computers and/or servers.

In some embodiments, the incubator 2 comprises an environmental sensor board that is able to detect a temperature, pressure, humidity and/or CO2 concentration within the incubator 2. In particular, the temperature values are able to be used to correct sensed pH, lactate, glucose, O2 or other temperature dependent values detected within the media/wells 24. Similarly, the atmospheric pressure and relative humidity values within the incubator 2 are able to be used to adjust O2 levels detected within the media/wells 24. In some embodiments, the system 10 is able to determine whether the incubator 2 door was opened (such that values during that time may be inaccurate) based on measured relative humidity and CO2 levels. In some embodiments, a smaller incubator is able to be used in which the slide, or slide and slider holder, is placed. In this case, the incubator can include a O2, CO2 port and modules for controlling temperature and humidity and pressure. The smaller sized incubator is able to be made from a transparent material such as glass or plastic.

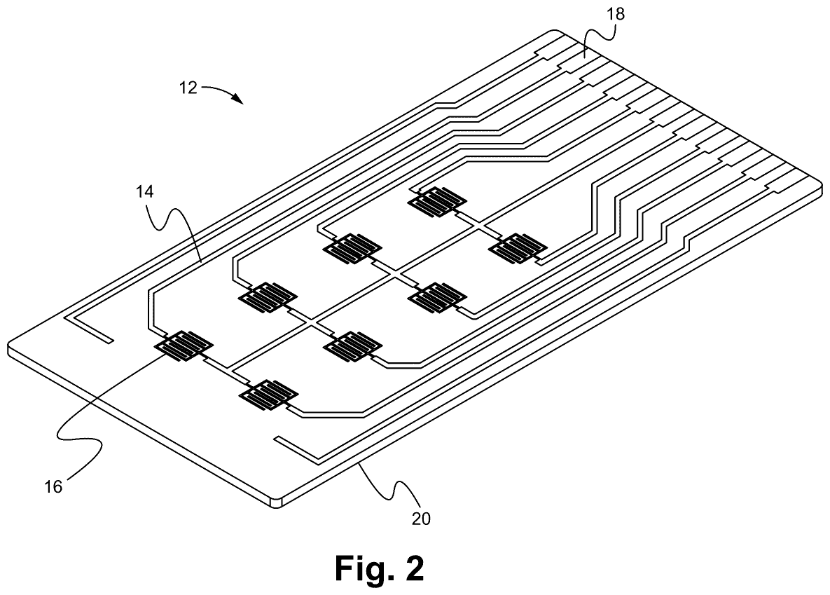

The polybiosensing-imaging system 10 is able to combine stand-alone disposable well slides 22 with disposable or non-disposable electrical interconnects substrates 12 and disposable or non-disposable electronics (e.g. sensor positioning assembly) each configured for placement and continuous use within the incubator 2. FIG. 2 illustrates a perspective view of an electrical interconnects substrate 12 according to some embodiments. The electrical interconnects substrate 12 includes a substrate 20 onto which a plurality of electrically conductive interconnects 14, electrodes 16, and external connectors 18 are formed. The interconnects 14 provide electrical interconnectedness between the electrodes 16 and the external connectors 18. The substrate 20 is able to be made of an optically transparent material, such as glass or plastic. The interconnects 14, the electrodes 16, and the external connectors 18 can be formed on a surface of the substrate 20. In some embodiments, the interconnects 14, the electrodes 16, and the external connectors 18 are printed onto the substrate 20. It is understood that the interconnects 14, the electrodes 16, and the external connectors 18 can be formed on the substrate 20 using any conventional techniques for forming and patterning electrically conductive material on a substrate including, but not limited to, photolithography. In some embodiments, the interconnects 14, the electrodes 16, and the external connectors 18 are made of gold, or an optically transparent material, such as ITO, silver ink, or carbon ink. Each electrode 16 can represent a single electrode or a plurality of connected electrodes, such as a plurality of interdigitated electrodes as shown in the circuit design of FIG. 2. The electrodes 16 are able to be used to measure impedance, pH, O2, glucose or other chemistries of the samples within the wells 24.

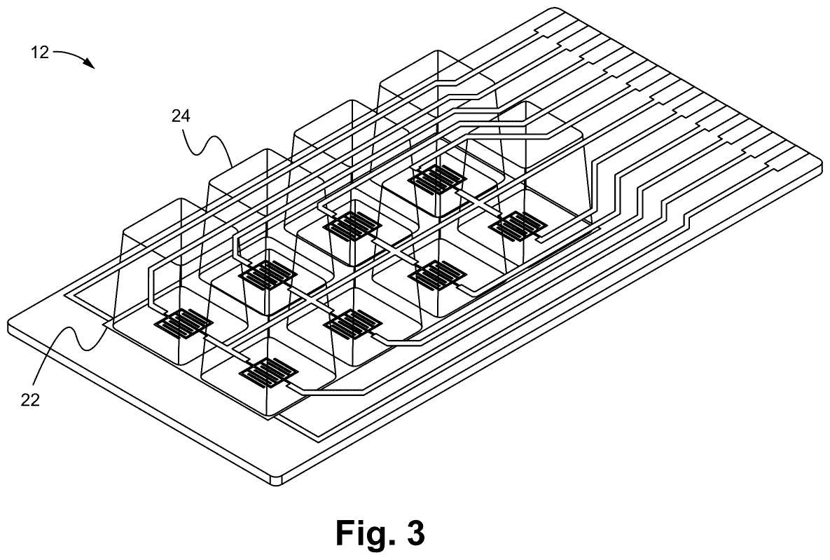

FIG. 3 illustrates a perspective view of a well slide 22 attached to the electrical interconnects substrate 12 in FIG. 2. The well slide 22 provides side wall and bottom wall structure for a plurality of wells 24. The well slide 22 is made of an optically transparent material, such as glass or plastic. The wells can be arranged in a variety of patterns and/or sizes, for example diameters and depths. For example, in some embodiments the wells 24 and/or well slide 22 are able to have one or more angled or tilted perimeter walls extending upward from a base (e.g. forming a partial pyramid, cone or other similar structure). In particular, instead of extending upward perpendicularly from the base, at least the outside facing walls of the wells 24 (e.g. walls not facing another well 24) are able to extend upward at an angle such that the veer toward a middle of the well 24. As a result, in such embodiments a perimeter around a top of the slide 22 (e.g. along the top of the outside facing walls of the wells 24) is larger than the perimeter around a bottom of the slide 22 (e.g. along the bottom of the outside facing walls of the wells 24) with a taper in perimeter size in between. This tapering is able to provide the benefit of enabling the slide 22 to be securely held in a desired position (e.g. with respect to optical probes and/or an imaging unit 36) by the sensor positioning assembly (described below). Alternatively, one or more of the walls of the wells 24 are able to be perpendicular to the base or outwardly angled away from a middle of the well 24.

In some embodiments, the wells 24 and/or slide 22 is able to include one or microfluidic channels through the bottom and/or walls of the wells 24 for introducing liquid and non-liquid samples and/or media culture to and retrieving liquid or non-liquid samples and/or media culture from the wells 24. In such embodiments, exchange mechanisms of the action can occur through a micro pump, microfluidic channels, passive mechanism by capillary force, connection through valves, manual use of a syringe or automated syringe exchanges powered by pumps. In some embodiments, the slide 22 is able to include one or more reservoirs for storing and introducing chemicals or drugs of interest to some or all of the wells 24 through one of the mechanisms described above via one or more channels or tubes. In some embodiments, the well slide 22 and/or wells 24 are able to include printed circuits for measuring pH, O2, glucose or lactate levels within the wells 24. In particular, the circuits are able to include electrodes that are treated with activators to facilitate the sensing of each analyte (wherein the surface treatment methods are able to include the use of different chemicals or biologics, such as, enzymes, proteins, antibodies, peptides, or other chemicals). These circuits are able to be printed on the inside of the bottom of the wells 24 and/or on an insert that is positioned within the wells 24. Further, the circuits are able to be controlled by and/or transmit measurements to the impedance printed circuit board 708, the flex circuit 706, the interconnect 12, other network devices 8, and/or a combination thereof.

It is understood that the well configuration shown in FIG. 3 is for exemplary purposes only, and that many alternative configurations are also contemplated. It is also understood that although the configuration shown in FIG. 2 shows wells having the same size and spacing, it is understood that slides can be configured with wells having different sizes and patterns. The exemplary well slide 22 has 2×4 array of individual wells 24. The well slide 24 and the electrical interconnects substrate 12 are configured such that at least one corresponding electrode 16 is aligned with each well 24. Although the configuration shown in FIGS. 2 and 3 shows a single electrode per well, it is understood that more than one electrode can be aligned with each well.

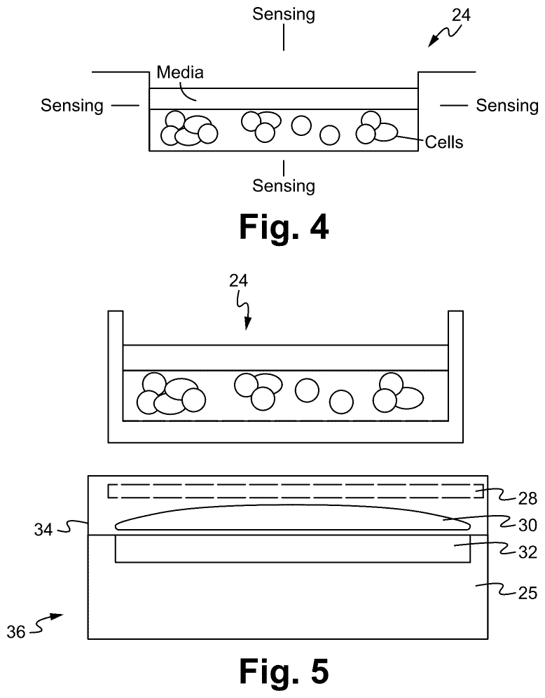

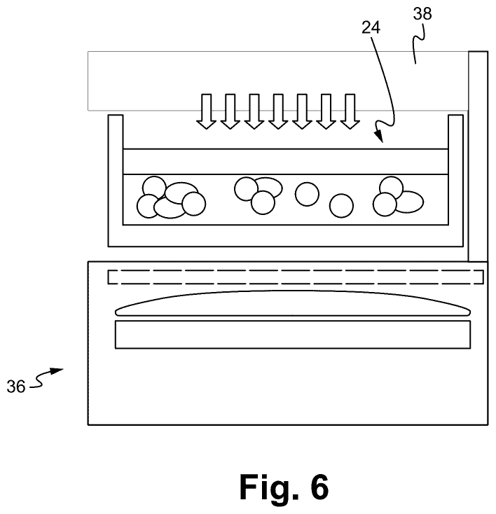

The polybiosensing-imaging system 10 is designed to sense multiple different characteristics of fluid sample within a given well. FIG. 4 illustrates a cut-out side view of an exemplary well having a biological sample to be sensed. The biological sample is suspended in a gel, and covered by a culture media. Sensing can be performed from above, below, and/or the sides of the well. For example, electrical sensing can be performed by electrodes (FIG. 2) included on the electrical interconnects substrate (FIG. 2) positioned under the bottom wall of the well. Additional electrodes can also be positioned on an interior and/or exterior surface of the well side walls. The electrodes enable impedance measuring. Optical sensing can be performed by optical interrogation, which may include providing illumination from the top of the well (FIG. 6), through the well side walls, and/or from the bottom of the well (FIG. 5), and sensing light by an optical reader positioned underneath the well bottom well. Acoustic sensing can be performed by attaching one or more acoustic sensors (not shown) on an interior and/or exterior surface of the well side walls and/or well bottom wall. Chemical sensing can be performed by attaching one or more chemical sensors (not shown) on an interior and/or exterior surface of the well side walls and/or well bottom wall. There are multiple use cases for which this is important. The cells are living organisms and modify their environment from a chemical perspective. It is important to be able to measure the change in chemistry that is caused by these cells. The chemistry to be measured can include, but is not limited to, pH, selective ions such as Chloride, Sodium, Potassium, Nitrate, Calcium, O₂, CO₂, cellular metabolites, reactive oxygen species (ROS), sugars and glucose, fat and other relevant chemistries such as secreted glycosaminoglycans and exosomes.

In some embodiments, the chemical sensors are ISFETs (ion-sensing field-effect-transistor) for measuring ion-concentrations in the fluid sample. The electrical signals output from the ISFETs can be translated to pH measurements. In other embodiments, the chemical sensors are chemical sensor dots whose color changes based on the chemistry they are exposed to. The chemical sensor dots can be interrogated optically. Similar to above, each chemical sensor dot can be tuned for sensitivity to one type of chemistry and for a specific range of concentrations. These chemical sensor dots can be printed on the bottom surface of a well and their color can be measured by the imaging unit. Chemical sensing can also be performed using electrochemical sensors, such as an integrated circuit configured to electrically measure chemical characteristics, for example pH and O2, and output a corresponding electrical signal. Such an integrated circuit has a sensing portion and an external connection pad. The integrated circuit can be positioned on the bottom or side wall of the well such that the sensing portion is immersed in either the fluid sample or culture media. The well can be adapted to include an electrical interconnect for coupling to the integrated circuit external connection pad, which in turn can be connected to an external electronic device, such as an imaging unit described below. Other types of sensors can be positioned on the well bottom and/or side walls and immersed within the fluid sample or the culture media to sense secretomes. Such a secretome sensor can include specific antibodies that attach to specific secretomes to be sensed, which when joined fluoresce or other modalities. This fluorescence can be sensed and measured using appropriate optical interrogation. It is understood that other sensing can be performed using alternative types of sensors. In some embodiments, sensors are not restricted to the interior surfaces of the well side and bottom walls, and can be inserted and suspended in either the biological sample or the culture media, as described in greater detail below.

The polybiosensing-imaging system 10 also is able to include non-disposable structure and/or electronics, which can be configured to perform optical interrogation of each well and related data capture, as well as provide electronic circuitry for implementation of impedance measuring using electrodes and acoustic measuring using acoustic sensors coupled to each well. The non-disposable electronics can include components such as a sensor positioning assembly (SPA), a reader, a filter, optical components, and a camera. The reader can also include additional control and processing circuitry, such as actuators and actuator control circuitry, system control, and data and image processors. The optical components can include any number of optical components configured to receive light from the well and to optically transmit the received light to an imaging sensor within the camera. The filter, the optical components, and the camera are collectively referred to as an imaging unit.

FIG. 5 illustrates a cut-out side view of an imaging unit according to some embodiments. The exemplary configuration shown in FIG. 5 is applied to a single well but can readily be applied to each well in a well slide. The imager or imaging unit 36 is able to include a reader 25, a filter 28, optical components 30, and a camera 32. Further, in some embodiments, the system 10 is able to include an imaging stage as described in detail below. The reader 25 is electrically coupled to the external connectors 18 (FIG. 2) of the electrical interconnects substrate 12 (FIG. 2). In some embodiments, the reader 25 includes pogo pins arranged to mate with the external connectors 18. In this case, the external connectors can be flat contact pads. In other embodiments, the external connectors 18 are configured as a plug or other type of adapter, and a connecting wire with appropriate configured adapter can be used to connect the external connectors 18 to the reader 25. The polybiosensing-imaging system 10 also is able to include a light source used to illuminate the interior of each well 24 and enable optical interrogation of the fluid sample stored therein. In some embodiments, a light source is positioned below the well 24, included as part of the camera 32. In other embodiments, the light source is positioned above the well, such as the light source 38 positioned over the well 24 in FIG. 6 or incorporated into the lid 26 as described below. In still other embodiments, the light source is positioned on the side or angular to (not shown). The light source can be in different form and type such as LED, laser, or light sheet.

In order to determine the color of any of the aforementioned sensors and/or capture images of the samples, these sensors and/or samples need to be illuminated. In some embodiments, the sensors and/or samples are illuminated by the light applied generally to the rest of the interior of the well, such as by the light source positioned below the well or above the well. In this case, reflectance characteristics of the sensors and/or samples are measured. If it is intended to measure the optical transmission characteristics of the sensors and/or samples, then a light source that illuminates the sensors from above is able to be used and the resulting transmitted light is sensed at the bottom side of each well 24. In other embodiments, light is directed through the well side walls onto the sensors and/or samples.

In the exemplary embodiment described above, the imaging unit 36 includes the filter, the optical components, the light source, and the camera. In general, the imaging unit 36 includes light sources, optical components, light sensors, and electronic circuitry used to generate and direct light into the well, and to receive and image resulting light, reflected and/or transmitted. The system 10 is able to include the imaging unit, electronic circuitry used to process image signals resulting from the sensed received light, mounting and movement mechanisms configured to move the imaging unit 36 to be directed as desired wells 24 and/or sensors, and electronic circuitry used to control the imaging unit 36 and the mounting and movement mechanisms. In some embodiments, the imaging unit 36 is able to comprise a second objective, optical fibers or an optical fiber bundle spaced one well 24 away from the first objective such that adjacent wells 24 are able to be sensed at the same time. In some embodiments, a deflection mirror or digital micromirror device (e.g. DLP microchip) is able to be positioned under each of the wells 24 in order to receive images from one or more of the wells 24.

In some embodiments, a partially silvered mirror is able to be used to transmit image data from the underside of the wells 24 to two locations. In particular, a portion of the light is able to be used for capturing images of the sample by the imaging unit 36 and a second portion of the light is able to be captured by one or more optical probes 704 (and/or a reader of the imaging unit 36) to interrogate/read the indicators (e.g. sensor dots). In some embodiments, one or more optical probes 704 are able to be co-mounted with a microscope objective of the imaging unit 36. In particular, the co-mounting with the object enables both the indicators and the sample images to be captured together. In such embodiments, a movement mechanism as described herein is able to align the co-mounted elements with the desired sensor/samples as needed. Alternatively, the movement mechanism is able to selectively move mirrors or other optical conduits for selectively directing light from the desired wells 24 to the imaging unit 36. In some embodiments, an objective can be used to create a virtual image of the bottom of the well(s) 24 at a desired location for excitation and readout of the sensors with low light loss. In particular, as long as the numerical aperture of the lens is large enough, the optical fibers (e.g. optical probes 704) are able to be placed at an image plane of the bottom of the well(s) 24.

The polybiosensing-imaging system 10 also is able to include a sensor positioning assembly (SPA) configured to attach to the well slide. The SPA enables one or more sensors to be precisely operably coupled with one or more of the wells 24 of the well slide 22. The SPA is configured such that the optical system including the imaging unit is still enabled to optically interrogate the wells.

The polysensing and imaging by the polybiosensing-imaging system 10 is able to occur for cells/samples in suspension or attached cells. In particular, cells/samples in suspension are able to be counted by different methods such as imaging, acoustic sensing, transmission light, light refraction, colorimetry and fluorometry. In some embodiments, images captured by the imaging unit 36 are able to be analyzed to count a quantity of suspended cells. In some embodiments, for each of the methods described herein, instead of counting a number of cells in the entire well 24 (using one or more of the methods), a number of cells in a sub-portion of the well 24 is able to be determined and then the number of cells in the entire well 24 calculated assuming a uniform distribution by multiplying the number in the sub-portion by the number of sub-portions that fit within the well 24. In some embodiments, acoustic sensing is able to be used to determine a cell count based on the real and imaginary part of the propagation constant of the sound waves. For example, the well 24 is able to be used as an acoustic resonant cavity and thus measure loss more sensitively by measuring resonant frequency and a quality factor Q of the resonator. Alternatively or in addition, the propagation characteristics are able to be used directly.

In some embodiments, transmission light is able to be used to determine a number of cells within a well 24 by using an etalon and measuring Q (where cells and the media having different optical properties (complex refractive index) and thus produce different wavelengths for measuring with the etalon). In particular, the ratio of cell volume over the whole well media volume is able to be used in combination with the wavelength measurements to determine an estimate of the number of cells. Alternatively, in some embodiments, the cells are able to be labeled with a contrast agent (e.g. fluorophore, monochromatic dye) such that the total light intensity from the well 24 is able to be measuring and the number of cells is able to be extracted by divided the total signal by the signal of one cell.

### Polybiosensing-Imaging System Including Pivotable Spa

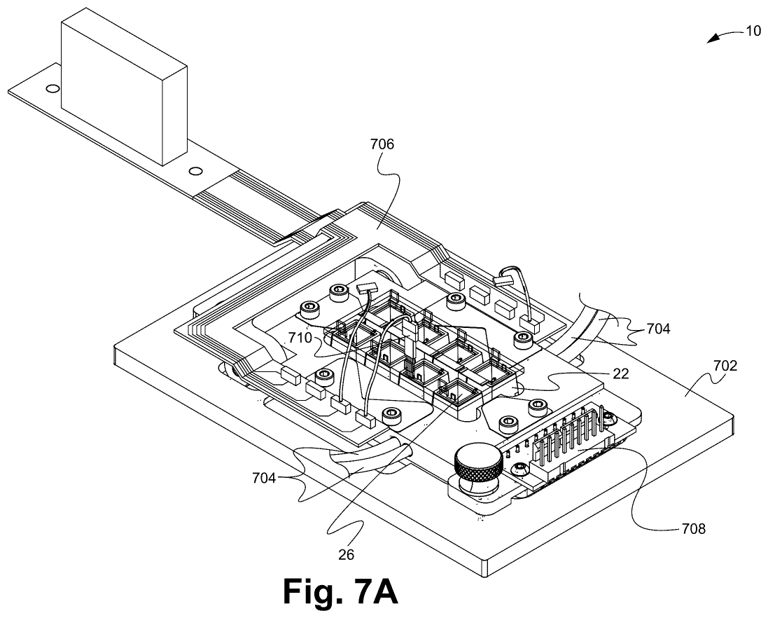

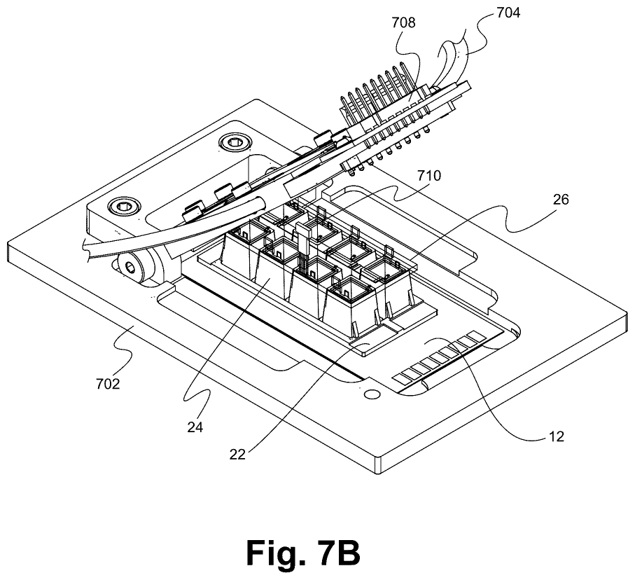

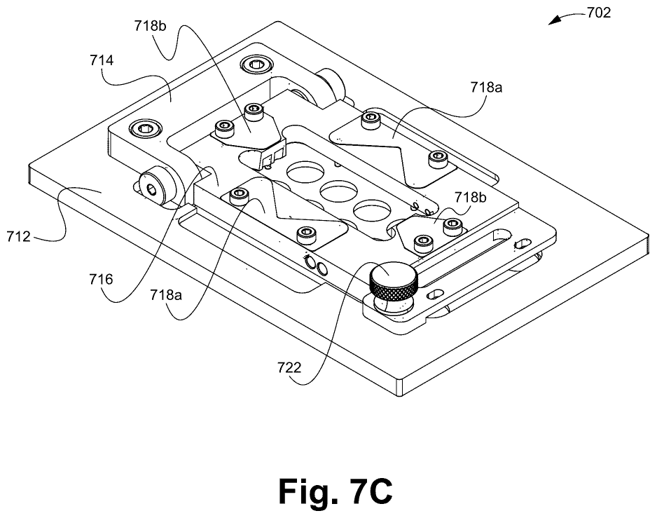

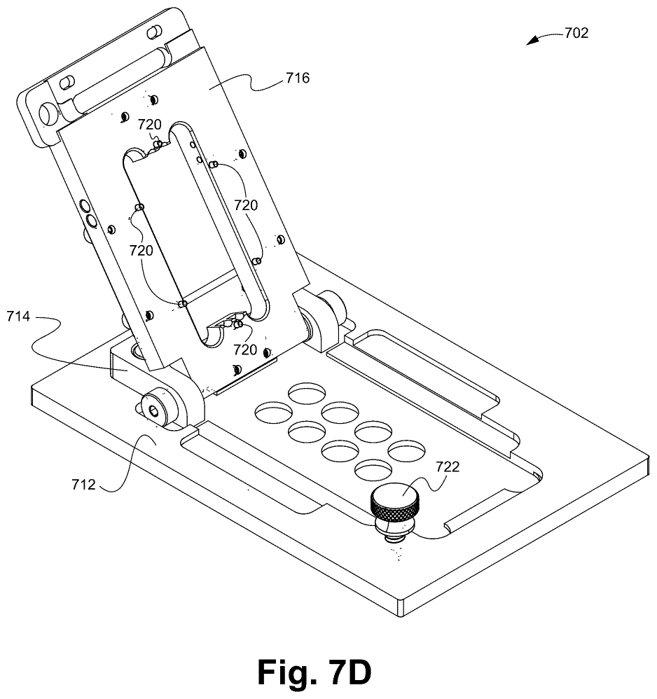

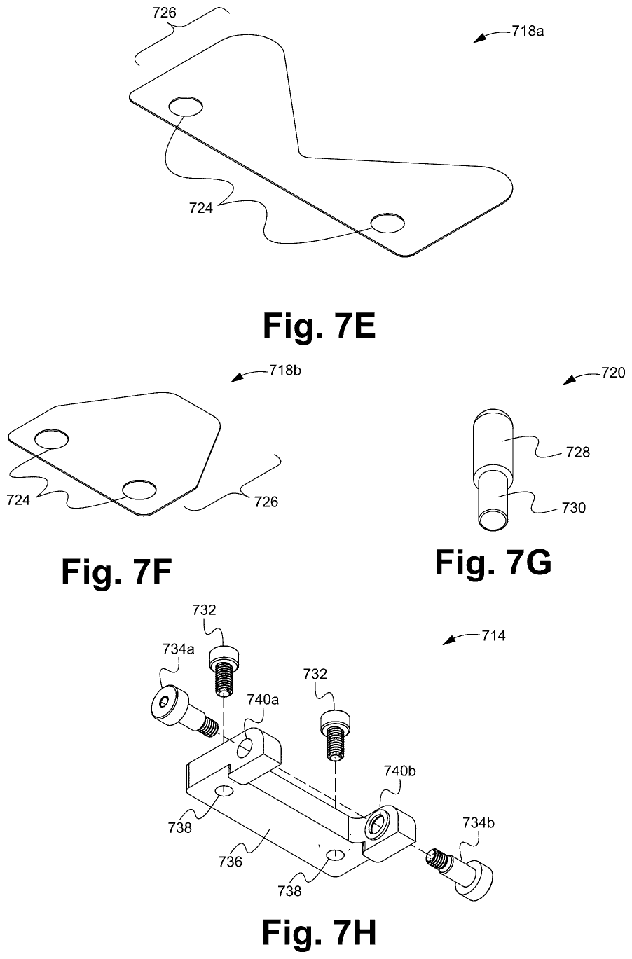

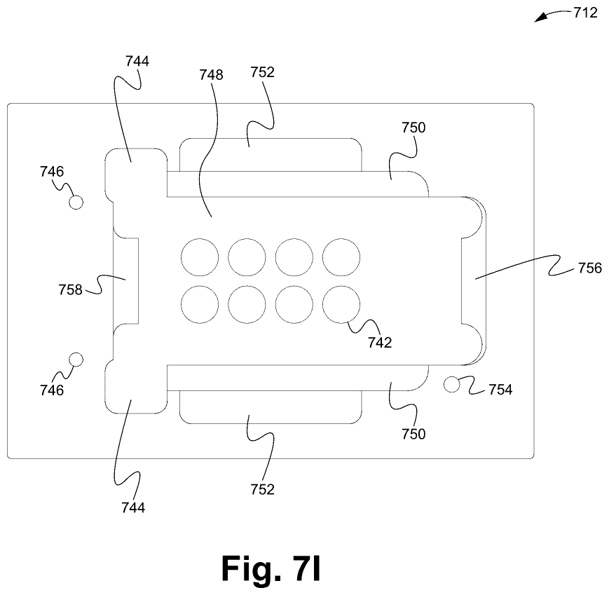

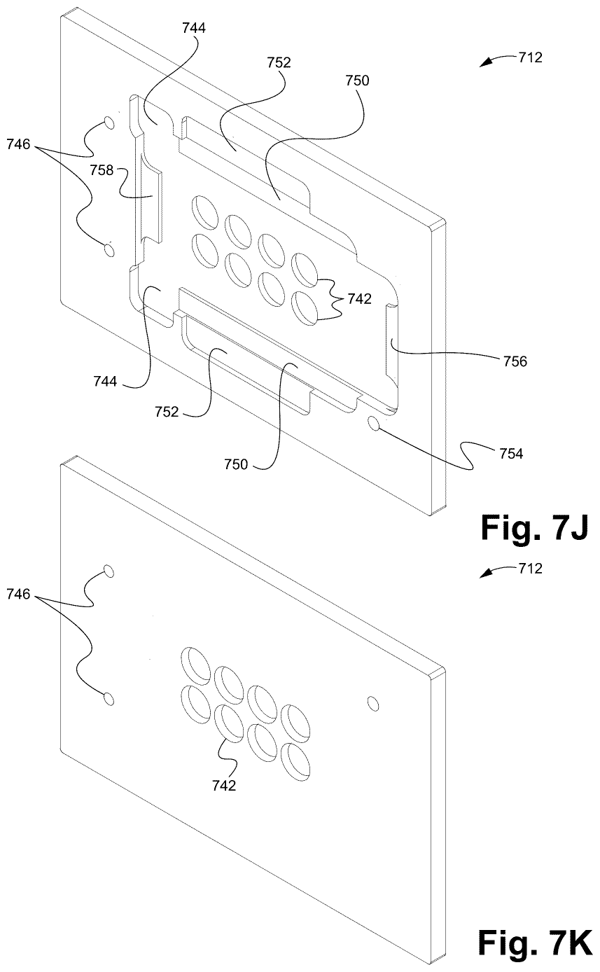

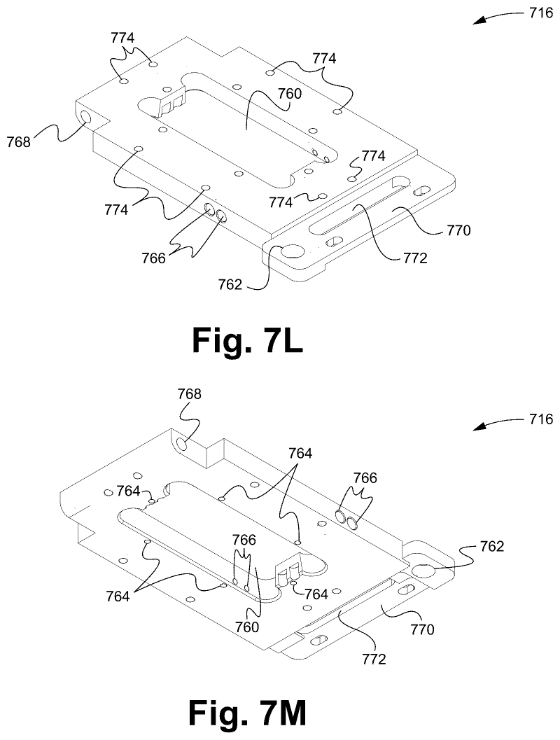

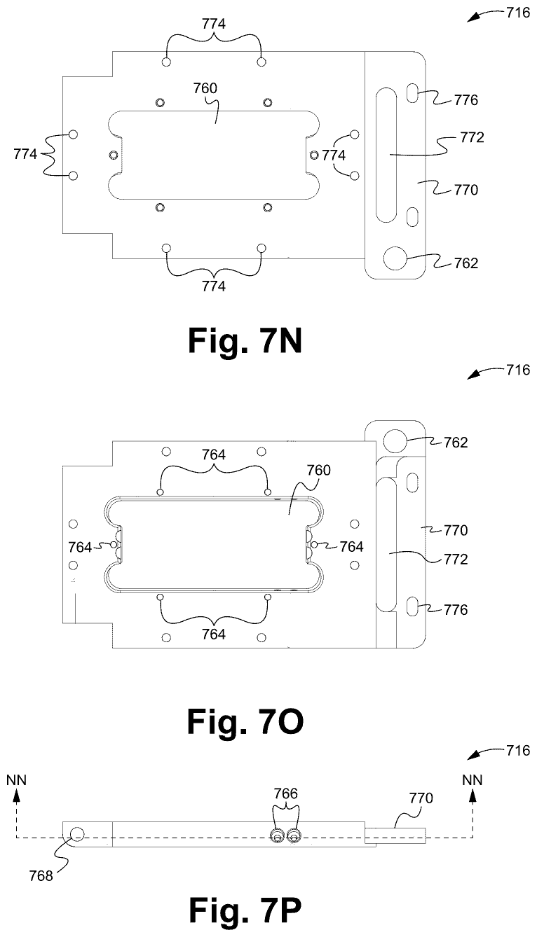

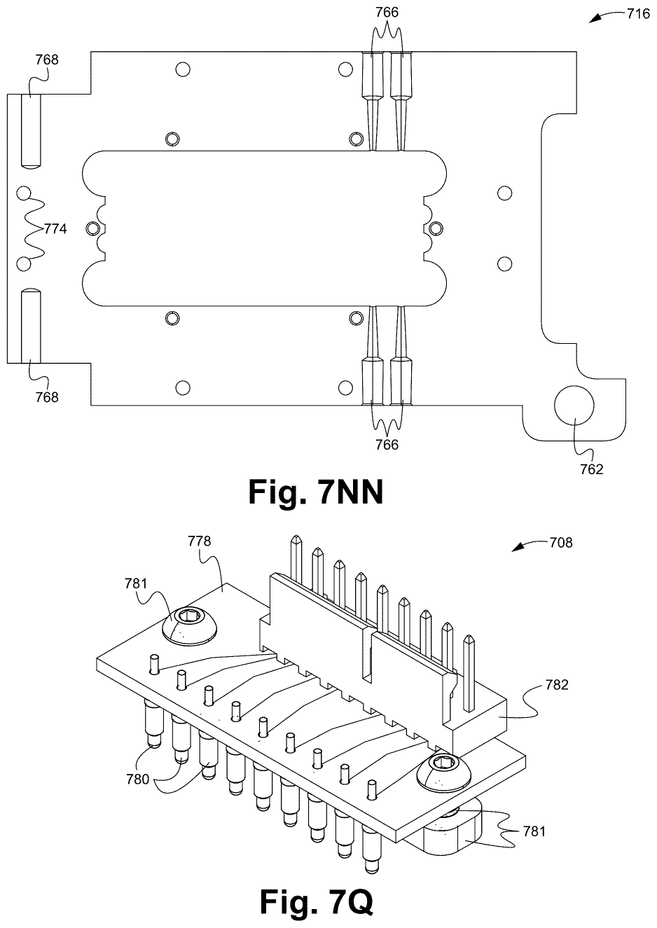

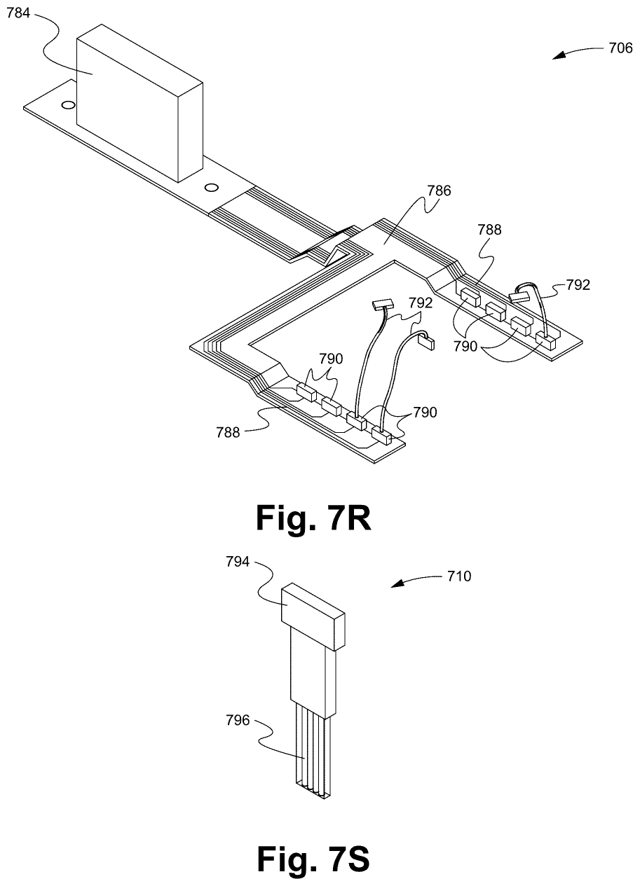

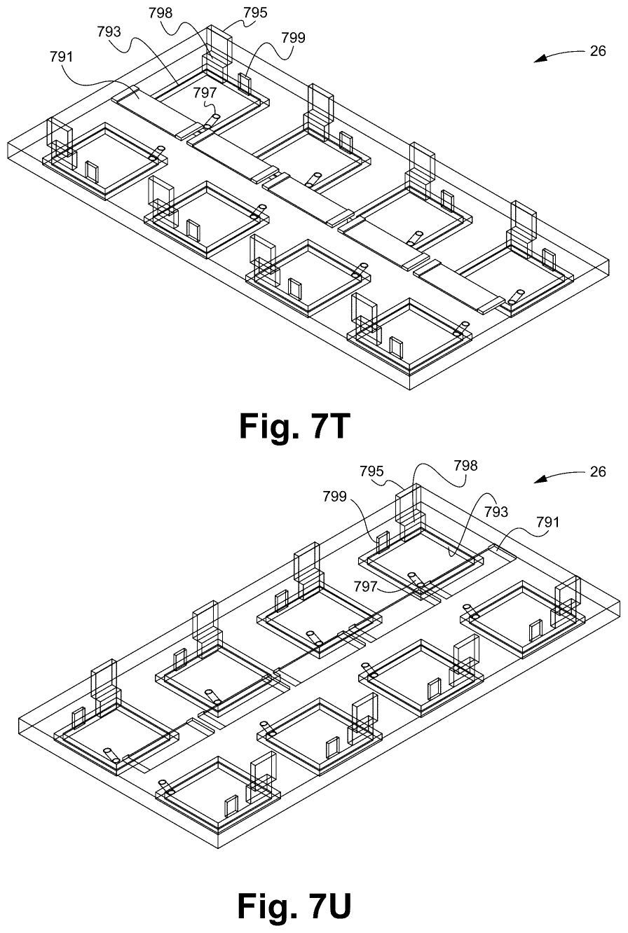

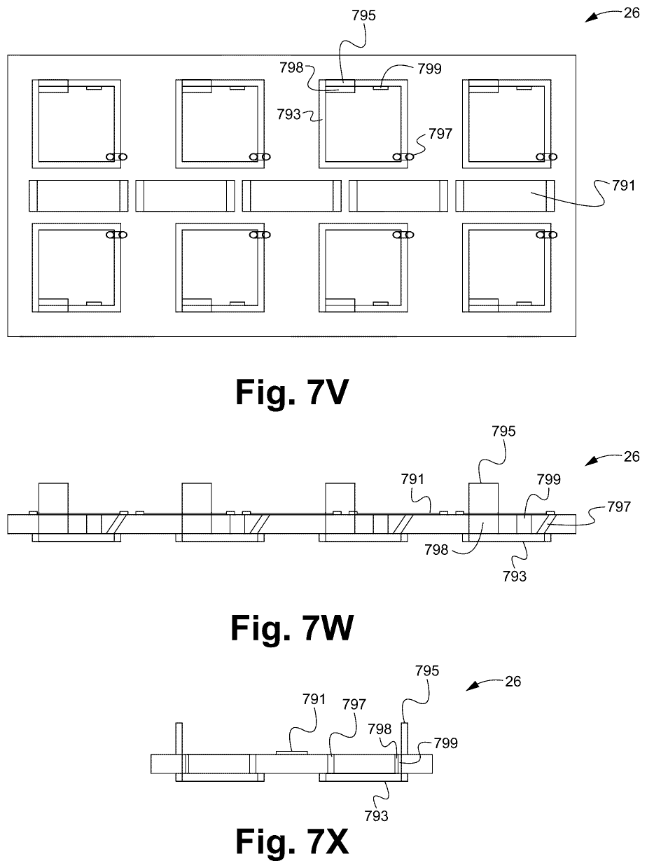

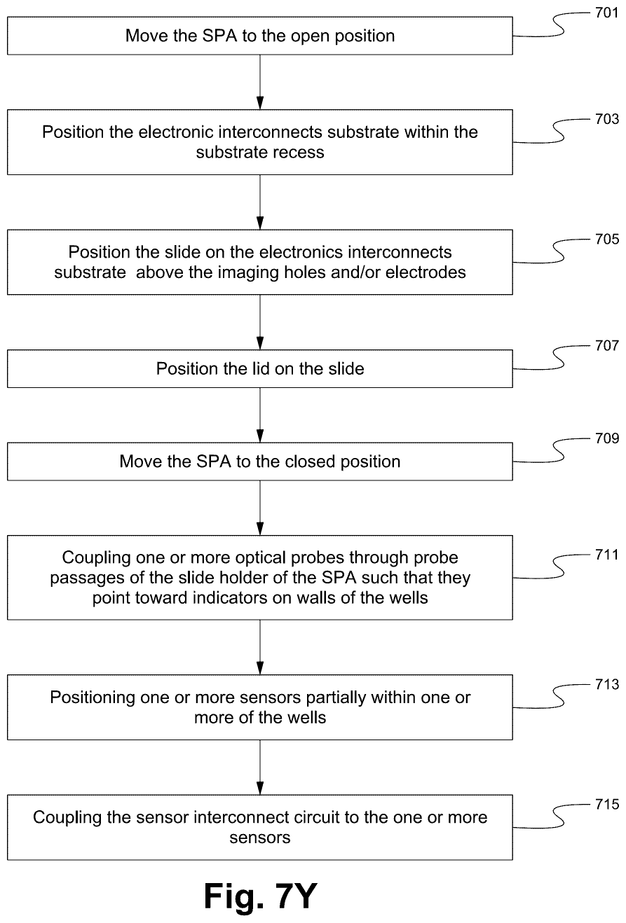

FIGS. 7A and 7B illustrate top perspective views of the polybiosensing-imaging system 10 with a sensor positioning system in a closed and open position, respectively, according to some embodiments. As shown in FIGS. 7A and 7B, the polybiosensing-imaging system 10 comprises a well slide 22 including a plurality of wells 24, a slide lid 26, an electrical interconnect substrate 12, an imaging unit 36 (not shown), a sensor positioning assembly 702, one or more optical probes 704 (e.g. light guides/light sensors), a sensor interconnect circuit 706 (e.g. flexible circuit), an impedance printed circuit board (PCB) 708 and one or more external sensors 710 (e.g. glucose sensors). Alternatively, one or more of the above components are able to be omitted. As described above, the imaging unit 36 is not shown for the sake of clarity, but is able to operably couples to (and/or be positioned adjacent to) a bottom of the SPA 702 for capturing images of the target sample within the wells 24 from underneath the wells 24 (through holes in a base plate of the SPA 702 (and the transparent electrical interconnects substrate 12) as described in detail below). Further, the imaging unit 36 is able to include multiple image capturing components for capturing images of the samples from the underside of each well 24, image capturing components that are able to capture images of the samples from the underside of multiple wells 24 at once, and/or movement mechanisms and associated electronics for moving image capturing components below each of the wells 24 in sequence for capturing images of the samples from below the wells 24.

The impedance PCB 708, the imaging unit 36, the optical probes 704 and/or the sensor interconnect circuit 706 are able to be coupled with the external electronic device 8 for receiving data detected from the wells/sample and/or transmitting control and/or excitation signals to the wells/sample.

As also shown in FIGS. 7A and 7B, the lid 26 is coupled on top of the well slide 22, which is positioned on top of the electrical interconnect substrate 12, all of which are held in place by a slide holder and base plate of the SPA 702 in a closed position. In particular, as described above, the well slide 24 and the electrical interconnects substrate 12 are configured such that at least one corresponding electrode 16 is aligned with each well 24. As a result, the electrodes 16 are able to be used to measure impedance of the target sample within the wells 24. Although the configuration shown in FIGS. 2 and 3 shows a single electrode 16 per well 24, it is understood that more than one electrode 16 can be aligned with each well 24.

The external sensors 710 are coupled with the sensor interconnect circuit 706 (e.g. via jacks) and are able to extend through the lid 26 into one or more of the wells 24 for measuring one or more characteristics of the samples within the wells 24. In particular, similar to the other sensors described herein, the external sensors 710 are able to be configured to detect one or more of pH, oxygen (O2), glucose, carbon dioxide (CO2), secretome, lactate (or other metabolites), mechanical sensing (e.g. via acoustic signals), temperature and/or any other chemistry that is able to be detected via amperometric/solid state sensors. In some embodiments, the cross-talk between different sensor types is able to be measured and calibrated. For example, it is possible that a sensor 710 for glucose might have a dependency on pH or O2 levels. In such cases, a calibration curve for the sensor 710 based on pH, O2 or other chemistry levels around the sensor 710 is determined and subsequently used to adjust the measured values of the sensor based on the pH, O2 or other chemistry levels around the sensor 710 at the time the value was measured.

The optical probes 704 are able to be detachably coupled through probe passages 766 of the slide holder of the SPA 702. Alternatively, the optical probes 704 are able to be permanently coupled through the probe passages of the slide holder (e.g. via glue, epoxy or other coupling mechanism). In either case, when the SPA 702 is in a closed position, the optical fibers of the optical probes 704 are each able to be adjacent to and pointed toward a side wall of one of the wells 24 for providing excitation optical signals to and/or detecting (e.g. fluoresced) optical signals from internal sensors within the wells 24. For example, the side walls of the wells 24 (or an insert within the wells 24) are able to include one or more indicators at which the optical probes 704 are directed for detecting one or more specific chemistries of the target sample within the well 24. In some embodiments, as described herein, the indicators printed on the wells and/or on an insert within the wells are implemented as a fluorescent dye (e.g. chemical sensor dots). The type of dye is selected so that a specific chemistry to be sensed, such as pH or O2, and selective ions attaches to the dye. The indicators/films/foils/dots are able to be read (sensed) with corresponding optical probes 704 positioned adjacent thereto via the SPA 702.

Alternatively or in addition, in some embodiments, imaging unit 36 is able to be used to measure from films/foils/dots placed on the side wall, top of bottom of the plate to visualize and quantify the signal from films/foils/dots. In some embodiments, the optical probes 704 are able to be clamped to the SPA 702 to relieve strain on the coupling between the probes 704 and the fiber passages when the SPA 702 moves between closed and open positions.

The optical probes 704 are able to be substantially similar to the light guides and/or light sensors described herein. For example, each optical probe 704 (e.g. light guide/waveguide/light sensor) is able to include an outer protective sheath surrounding one or more optical fibers. At least a proximate end of the optical fibers is able to protrude through the probe passages such that the fibers are adjacent to one or more indicators/sensors of the wells 24. The distal end of optical fibers is able to be operably connected to a measurement device that can emit light at specific wavelengths (e.g. produce excitation signals for transmission to sensors within the wells 24). As a result, a fluorescence process can be used in which the measurement device directs a first wavelength of light, via the optical fiber, to the indicator/sensor within the well 24, which results in a fluorescent emission by the indicator/sensor when in the presence of and/or based on the associated chemistry within the well 24. The fluorescent emission is able to be at a second wavelength different than the first wavelength. The proximate end of the optical fibers receives and transmits the fluorescent emitted light back to the measurement device at their distal ends. Alternatively, one or more of the probes 704 can include two or more separate optical fibers, one or more optical fibers configured to emit light at the first wavelength to the indicator/sensor, and one or more other optical fibers configured to receive and transmit the fluorescent emitted light back to the measurement device. By way of example, the range of excitation wavelength (first wavelength) for O2 sensing is 500-650 nm and the range of emission wavelength for O2 sensing is 575-775 nm, and the range of excitation wavelength (first wavelength) for pH sensing is 460-650 nm and the range of emission wavelength for pH sensing is 525-700 nm.

In some embodiments, one or both of the distal and proximate ends of the optical fibers of the probes 704 extend beyond the outer sheath. In some embodiments, the outer sheath is able to comprise a transparent material that minimizes, if not prevents, optical occlusion due to the probe 704. Minimizing, if not preventing, optical occlusion within the well 24 helps enable simultaneous multi-modal functionality, such as simultaneous use of the probes (chemical sensing) and optical interrogation of the well interior (optical sensing). In some embodiments, as described above, the probes 704 are able to be operably coupled with the external electronic device 8. In this manner, data signals output from the sensor/indicator is able to be communicated to the external electronic device 8 for further processing or routing. Various types of sensors, such as voltage sensitive dye, can be used instead of fluorescence based sensor for chemical sensing.

The impedance PCB 708 is able to be coupled to a PCB platform of the slide holder of the SPA 702 and electrically detachably coupled or decoupled with the external connectors 18 based on whether the SPA 702 is in the closed or open position (e.g. via one or more pogo pins). Further, the impedance PCB 708 is able to be operably coupled with the external electronic device 8 (e.g. via a network interface) for controlling and/or receiving impedance indicating signals from the electrical interconnects substrate 12. As a result, the impedance PCB 708 is able to provide impedance data of the wells 24 to the electronic device 8 and/or receive commands from the electronic device 8 based upon which the impedance is measured. Additionally, in some embodiments the electrodes 16, the substrate 12 and/or the impedance PCE 708 are able to be used to measure other characteristics of the target sample within the wells 24. Specifically, electrical based sensors of other chemistries are able to be used and in communication with the electrodes 16 such that the electrodes 16 are able to be used to measure the other chemistries as desired.

FIGS. 7C and 7D illustrate top perspective closed and open views, respectively, of the SPA 702 according to some embodiments. As shown in FIGS. 7C and 7D, the SPA 702 comprises a base plate 712, a hinge mechanism 714, a slide holder 716, one or more guide plates (e.g. double prong 718a, single prong 718b), one or more push rods 720 and a lock bolt 720. Alternatively, one or more of the base plate 712, the hinge mechanism 714, the slide holder 716, the one or more guide plates (e.g. double prong 718a, single prong 718b), the one or more push rods 720 and/or the lock bolt 720 are able to be omitted. The hinge mechanism 714 is coupled to the base plate 712 and the slide holder 716 and enables the slide holder 716 to pivot about the hinge mechanism 714 with respect to the base plate 712 between an open position where the slide holder 716 is angled away from the base plate 712 (see FIG. 7D) and a closed position where the slide holder 716 is adjacent and/or parallel to the base plate 713 (see FIG. 7C). The lock bolt 720 is able to detachably couple to a lock bolt aperture 754 (e.g. external threads of the lock bolt 720 threaded into internal threads of the lock bolt aperture 754) in order to hold/lock the SPA 702 in the closed position. In particular, as described below, in some embodiments the push rods 720 are able to provide a spring force that resists the closing of the SPA 702 such that the lock bolt 720 is able to force at least partial compression of the push rods 720 when securing the slide holder 716 in the closed position.

As shown in FIGS. 7E and 7F, the guide plates 718a, 718b each comprise one or more coupling holes 724 and one or more prongs 726. As shown in FIG. 7C, the guide plates 718a, 718b are able to be coupled to a top of the slide holder 716 (e.g. via the coupling holes 724 and a plurality of screws) such that their prongs 726 extend inwards above a slide window of the slide holder 716 configured to receive a slide 22 when in the closed position. In particular, as SPA 702 is moved from the open to the closed position, the slide 22 moves through the slide window of the slide holder 716 such that the prongs 726 of the guide plates 718a, 718b contact the outward facing walls of the wells 24 of the slide 22 and thereby guide the slide/wells into the correct position within the SPA 702. This guiding is able to be further facilitated in embodiments where in the outer walls of the wells 24 are inwardly angled such that the contact between the guides 718a, 718b and the outer walls causes the wells/slide to move toward the correct position.

The push rods 720 are coupled to a bottom of the slide holder 716 such that they protrude from the bottom of the slide holder 716 and contact the base plate 712, slide 22 and/or electronic interconnects substrate 12 when the SPA 702 is in the closed position. Specifically, as shown in FIG. 7G, the push rods 720 comprise a head 728 and a stem 730 and as shown in FIG. 7D, the heads 728 of the push rods 720 are able to fit within rod apertures of the slide holder 716 such that the stems 730 protrude downward from the bottom of the slide holder 716. As a result, the push rods 720 prevent the slide holder 716 from closing too much and damaging the slide 22 and/or substrate 12. Further, in some embodiments the head 728 is able to be hollow and/or include a spring such that when a compression force is applied to the stem 730 the stem 730 is able to telescope into the head 728 as resisted by the spring. As a result, when coupled with the slide holder 716, the push rods 720 are able to provide a springing/flexible/elastic downward force the prevents the slide holder 716 from being clamped down too much and/or contacts the top of the slide 22 and/or substrate 12 thereby holding the slide 22 and/or substrate 12 in place. Indeed, the push rods 720 improve image and measurement stability and planarity despite the opening/closing or other movement of the SPA 702. Although as shown in FIG. 7D, the SPA 702 comprises six push rods 720 positioned around the slide window, more or less push rods 720 positioned in different and/or the same positions on the bottom of the slide holder 716 are able to be used.

FIG. 7H illustrates a perspective exploded view of the hinge mechanism 714 according to some embodiments. As shown in FIG. 7H, the hinge mechanism 714 includes one or more hinge fasteners 732, one or more axles 734 a, b and a hinge base 736 having one or more coupling holes 738 and one or more axle holes 740 a, b. The hinge base 736 is coupled to the base plate 702 by the fasteners 732 and the coupling holes 738 (e.g. with the fasteners threaded into threaded holes of the base plate). The axle 734a is positioned through a first of the axle holes 740a and at least partially into a first end of the axle channel 768 of the slide support 716 and the axle 734b is positioned through a second of the axle holes 740b and at least partially into a second end of the axle channel 768 such that the slide support 716 is able to rotate about the axles 734 a, b between the open and closed positions.

FIGS. 7I, 7J and 7K illustrate top, top perspective and bottom perspective view, respectively, of the base plate 712 according to some embodiments. As shown in FIGS. 7I-K, the base plate 712 comprises one or more imaging holes 742, a hinge recess 744, one or more hinge coupling apertures 746, a substrate recess 748, a slide support recess 750, a probe recess 752 and a lock bolt aperture 754. As described above, the lock bolt aperture 754 is able to have internal threads for coupling with the lock bolt 722. The hinge recess 744 is able to receive a bottom of the hinge mechanism 714 such that the hinge fasteners 732 are able to couple with the hinge coupling apertures 746 (e.g. thread together) in order to secure the hinge mechanism 714 to the base plate 712. The substrate recess 748 is able to be configured to receive the electronics interconnect substrate 12 with an upper ledge 756 abutting a first end of the substrate 12 and a lower ledge 758 abutting a second end of the substrate 12 (thereby holding the substrate 12 within the recess 748. Similarly, the slide support recess 750 and the probe recess 752 are configured to receive at least a bottom of the slide support 716 and the optical probes 704, respectively.

Lastly, the imaging holes 742 are able to be positioned such that they each align with a bottom of one of the wells 24 when the slide 22 is coupled within the slide holder 716 (e.g. the SPA 702 is in the closed position). Indeed, this enables the imaging unit 36 to capture images and/or other data from the samples within the wells 24 from underneath the base plate 712 via the holes 742. In particular, these imaging holes 742 provide the advantage of reducing optical interference and the associated loss caused by the optical properties of transparent base plates (e.g. glass base plates without holes) as wells as eliminating issues with debris on the base plate occluding vision of the samples.

FIGS. 7L, 7M, 7N, 7NN, 7O and 7P illustrate top perspective, bottom perspective, top, top cross-sectional, bottom and side views, respectively, of the slide holder 716 according to some embodiments. As shown in FIGS. 7L-P, the slide holder 716 comprises a slide window 760, a lock bolt channel 762, one or more push rod slots 764, one or more probe passages 766, a hinge passage 768, a PCB platform 770, a PCB window 772 and one or more guide plate holes 774. Although as shown in FIGS. 7L-P, the slide holder 716 comprises four probe passages 766 and six push rod slots 764, more or less probe passages 766 and/or push rod slots 764 are contemplated. In particular, in some embodiments the slide holder 716 has two or more probe passages 766 for each of the wells 24 of the slide 22 configured to fit within the slide window 760 (with each of the passages 766 being positioned adjacent to an outer facing wall of one of the wells 24).

The slide window 760 is able to be sized to fit around the slide 22 with the guide plates 718a, 718b coupled to the top of the slide holder 716 (via the guide plate holes 774 and fasteners/screws) and protruding over at least a portion of the top of the slide window 760 for contacting the outside walls of the slide 22. The lock bolt channel 762 is configured to receive as shaft, but block the head of the lock bolt 722 such that when the lock bolt 762 extends through the channel 762 to couple with the lock bolt aperture 754, the lock bolt 762 applies a closing/downward force on the slide holder 716 thereby holding it in the closed position. The push rod slots 764 are configured to receive the heads 728 of the push rods 720 such that the stems 730 protrude down from a bottom of the slide holder 716 (as described above). The push rod slots 764 are able to be positioned in any location on the bottom of the slide holder 716 and in any quantity as desired for locations and quantities for the push rods 720.

The probe passages 766 are able to extend from a side of the slide holder 716 into the slide window 760 such that when a slide 22 is within the window 760, the inside of the probe passages 766 face a side of one of the wells 24 of the slide 22 (e.g. at a location of a sensor/dot within the wells 24). As described above, the probe passages 766 are sized to receive an end portion (e.g. one or more optical fibers and/or protective sheaths) of the probes 704 such that the probes 704 fit through the passages 766 and face a side of one of the wells 24 of the slide 22 (e.g. either flush with or partially protruding out of the inner opening of the passage 766). In some embodiments, as shown in the top cross-sectional at line NN of FIG. 7P view of the slide holder 716 of FIG. 7NN, the passages 766 are able to narrow in diameter from their outer opening to their inner openings such that the portion of the probes 704 at or extending from the inner openings is more precisely pointed at the side of one of the wells 24 (e.g. at a location of a sensor/dot within the well 24). In such embodiments, while a large diameter of the probe 704 is able to extend through the first larger diameter portion of the passage 766, after a narrowing point or portion of the passage 766, only smaller diameter of the probe 704 (e.g. the one or more central optical fibers) extends further through the narrowed portion of the passage 766 to the end of the passage 766 proximate one of the wells 24. Alternatively, the passages 766 are able to have the same diameter from the outer opening to the inner opening.

The hinge passage 768 is configured to receive the axle 734 of the hinge mechanism 714 such that the slide holder 716 is able to pivot about the hinge mechanism 714 (and couple with the base plate/hinge mechanism). The PCB platform 770 is configured to couple to the impedance PCB 708 via PCB coupling mechanism 781 and the PCB platform apertures 776. Further, the PCB platform 770 is able to include the PCB window 772 such that electrical couplers 780 (e.g. pogo pins) are able to extend from the PCB 708 as coupled to the platform 770 through the PCB window 772 and electrically couple with the external connectors 18 of the substrate 12 when the SPA 702 is in the closed position.

FIG. 7Q illustrates a top perspective view of the impedance PCB 708 according to some embodiments. As shown in FIG. 7Q, the impedance PCB 708 comprises a coupling mechanism 781 and a printed circuit 778 that is electrically coupled between one or more electrical couplers 780 (e.g. pogo pins or other electrical coupling mechanism known in the art) and a network interface 782. Specifically, the impedance PCB 708 is configured to electrically couple to the electrodes 16 of the substrate 12 (via the external connectors 18) and electrically couple with external devices 8 (via the network interface 782) thereby enabling the electrodes 16 to be controlled by the devices 8 and the impedance or other well data measured/detected by the electrodes 16 to be transmitted to the devices 8 for analysis. Additionally, as described above, the coupling mechanism 781 is able to couple the impedance PCB 708 to the PCB platform 770 via the PCB platform apertures 776 (with the electrical couplers 780 extending through the PCB window 772 and electrically coupling with the external connectors 18 of the substrate 12 when the SPA 702 is in the closed position).

In some embodiments, the electrical couplers 780 comprise a plurality of electrical coupling components (e.g. pins) that are each coupled to the electrodes 16 via the external connectors 18. In some embodiments, two or more of the electrical coupling components are electrically coupled to each of the external connectors 18 in order to improve the electrical connection between the PCB 708 and the electrodes 16. In particular, this provides the advantage of being able to use the signals received from the two or more components coupled to each connector 18 to determine an average signal, identify noise and multi-check measurement integrity, as well as lowering connection resistance between the PCB 708 and the electrodes 16. In some embodiments, the impedance PCB 708 is able to further electrically couple with the lid 26 in order to control heating resistors of the lid 26 (and thus the temperature of the wells 24 under the lid 26). This coupling is able to be via the substrate 12 or directly from the PCB 708. Alternatively, the heating resistors of the lid 26 are able to be controlled by the substrate 12 and/or otherwise controlled via a separate circuit.

FIG. 7R illustrates a top view of the sensor interconnect circuit 706 according to some embodiments. As shown in FIG. 7R, the interconnect circuit 706 comprises a network interconnect 784, a forked circuit substrate 786 having a plurality of tines 788, a plurality of sensor adapter jacks 790 and a plurality of sensor adapters 792. Although as shown in FIG. 7R, the circuit 706 includes eight jacks 790 and adaptors 792 more or less jacks 790 and/or adaptors 792 are able to be used. In particular, in some embodiments each tine 788 includes at least one jack 790 and at least one adaptor 792 for each of the wells 24 adjacent to that tine 788. The forked circuit substrate 786 provides an electrical connection between each of the sensor adaptor jacks 790 positioned on the tines 788 and the network interconnect 784 (e.g. via a plurality of traces). The sensor adapters 792 are configured to electrically couple between the sensors 710 and the sensor adapter jacks 790 such that sensors 710 are able to be controlled and sensor data is able to be read from the sensors 710. Specifically, the network interconnect 784 is able to be operably coupled with the devices 8 such that the devices 8 are able to send commands for controlling the sensors 710 through the interconnect 784, the circuit substrate 786, the jacks 790 and the adaptors 792 to the sensors 710. Similarly, the adaptors 792 are able to receive and transmit sensor data (e.g. glucose sensor data) from the sensors 710 to the devices 8 via the jacks 790, substrate 786 and interconnect 784. In some embodiments, the forked circuit substrate 786 is a flexible substrate. Alternatively, the forked circuit substrate 786 is able to be a rigid substrate. The forked configuration of the substrate 786 provides the benefit of enabling the jacks 790 and the adaptors 792 (and thus the sensors 710) to be directly adjacent to each well 24 that needs to be sensed. In some embodiments, the sensor interconnect circuit 706 is able to further electrically couple with the lid 26 in order to control heating resistors of the lid 26 (and thus the temperature of the wells 24 under the lid 26).

In some embodiments, the sensor adaptor jacks 790 and the network interconnect 784 are encapsulated in epoxy, the forked circuit substrate 786 and sensor adaptors 792 have a conformal coating and/or dielectric grease (e.g. Dow Molykote) is applied to the connection between sensors 710 and the adaptors 792. In particular, these steps provide the advantage of providing an insulation resistance in excess of 1 G ohm in order to keep a leakage current of the sensors 710 and/or the sensor interconnect circuit 706 acceptably low. In some embodiments, the impedance PCB 708 is able to be incorporated into the sensor interconnect circuit 706 such that the sensor interconnect circuit 706 detected and/or controlled impedance measurements. In some embodiments, the sensor interconnect circuit 706 is able to comprise one or more temperature sensors to measure well 24 temperature from inside the well 24.

In some embodiments, all of the electrodes (and other electrical interconnections) of the sensors 710 or other components (e.g. inserts) within the wells 24 are able to be optically transparent and/or have similar or the same electrochemical potentials so as to not unintentionally drive additional (non-equilibrium) reactions within the fluid (e.g. electrolyte) within the wells 24. With this in mind, in some embodiments the electrodes are made of gold and/or platinum. In particular, because gold and platinum have very similar electrochemical potentials, proper electrical potential equilibrium within the well fluid is able to be maintained. Alternatively, other conductive materials are able to be used such as, but not limited to, poly(3,4-ethylenedioxythiophene): poly(styrenesulfonate) (PEDOT), polyphenylene oxides (PPOS), polyanilines, fluorine-doped tin oxides (FTO), carbon nanotubes or a conductive mesh. In some embodiments, all of the electrodes (and other electrical interconnections) of the sensors 710 or other components (e.g. inserts) within the wells 24 are able to be passivated against non-specific absorption of chemicals or cells by applying silicon dioxide paint on all electrical interconnections and traces.

FIG. 7S illustrates a perspective view of a sensor 710 according to some embodiments. As shown in FIG. 7S, the sensor 710 comprises a connection jack 794 for coupling with one of the adaptors 792 and a sensor tip 796 configured to fit through a sensor hole of the lid 26 and detect a target chemistry (e.g. glucose, O2, or other chemistry described herein) within one of the wells 24. In some embodiments, the tip 796 is sized to fit within a larger sensor hole of the lid 26. Alternatively, the tip 796 is able to be sized to fit within a smaller sensor hole of the lid 26.

FIGS. 7T, 7U, 7V, 7W and 7X illustrate top perspective, bottom perspective, top, front and side views of the lid 26, respectively, according to some embodiments. As shown in FIGS. 7T and 7U, the lid 26 comprises one or more large sensor holes 798 for each well 24, one or more small sensor holes 799 for each well 24, one or more angled needle holes 797 for each well 24, one or more sensor supports 795 for each well 24, a well rim guide 793 for each of the wells 24 and a temperature control circuit 791. As a result, the lid 26 is able to facilitate the removal or addition of sample and/or culture media from each of the wells 24 via the holes described above and/or additional channels or microchannels through the lid 26. Alternatively, one or more of the large sensor holes 798, the small sensor holes 799, the angled needle holes, the sensor supports 795, the well rim guides 793 and the temperature control circuit 791 are able to be omitted. Further, although as shown in FIGS. 7T and 7U, the lid 26 includes one small hole 799, one large hole 798, one angled hole 797, one sensor support 795 and one well rim guide 793 for each well 24 of the slide 22, more of one or more of the above components for each well is contemplated.

The large and/or small sensor holes 798, 799 are able to be positioned along an outer edge of the lid 26 such that they are positioned over an outward most facing portion of the inside of the well 24. As a result, any sensors 710 inserted into the holes 798, 799 protrude down a side or non-central portion of the well 24 and thus not interfere with the capture of images of the sample within a middle portion of the well 24. Further, by being adjacent to the outer edge of the lid 26, the sensor holes 798, 799 minimize the distance between the sensors 710 (when inserted in one of the holes 798, 799) and the sensor interconnect circuit 706. The sensor supports 795 are able to extend above the lid 26 adjacent to one or more of the holes 798, 799 in order to provide structural support to the sensors 710 inserted into the adjacent hole 798, 799. Indeed, by stabilizing the sensors 710, the sensor supports 795 reduce any shadows produced by the sensors 710 as well as keeping the sensors 710 out of the optical path (e.g. the central portion of the wells 24). In some embodiments. the sensors 710 are able to be coupled to the sensor supports 795 via one or more adhesives (e.g. epoxy).

The angled syringe holes 797 are able to be positioned along a central portion of the lid 26 such that they are positioned above an inward most facing portion of the inside of the well 24. In particular, the top opening of the holes 797 is able to be above a top inside corner of the inside of one of the wells 24 and the holes 797 are able to be angled toward the bottom inside corner of the inside of the one of the wells 24. As a result, a straight syringe inserted into one of the holes 797 will remain adjacent to the inside of a wall of the well 24 and extend from a top corner of that wall to the opposite bottom corner of the same wall. Indeed, this provides the benefit of both preventing the syringe from occluding a center of the well 24 and preventing the syringe from damaging any other sensors 710, sensor dots/indicators (e.g. printed on the well 24 or an insert), electrodes or other components that are positioned along the bottom and/or the opposite wall of the well 24 (and/or in the middle of the well 24). Further, by enabling a syringe to be inserted into each well 24, the angled needle holes 797 enable the withdrawing of samples, adding of cells or drugs and/or adding or replacing of liquid media. In some embodiments, the lid 26 further comprises one or more syringe hole plugs that are each able to plug one of the syringe holes 797 thereby preventing the condensation of water (while still allowing CO2 exchange). This would provide the benefit of preventing evaporation so that there does not need to be high humidity for the cell culture within the wells 24.

The well rim guides 793 are able to slightly protrude from a bottom of the lid 26 and have a profile shape that matches but is slightly larger or slightly smaller than the profile of the top of the wells 24. As a result, when the lid 26 is positioned on the slide 22 of wells 24, the top of each of the wells 24 is able to slide within one of the well rim guides 793 (if the rim guide 793 is slightly larger than the top of the wells 24) or each of the well rim guides 793 is able to slide within the top of one of the wells 24 (if the rim guide 793 is slightly smaller than the top of the wells 24). Thus, because in such a position the rim guides 793 surround the outside or inside of the top of the wells 24, the rim guides 793 help prevent the lid 26 from sliding or otherwise misaligning with the top of the wells 24 and/or the slide 22. The temperature control circuit 791 is able to comprise one or more resistors that when subject to an electric current provide heat to the lid 26 and thus the wells 24 below the lid 26. This temperature control provides the benefit of helping to prevent condensation on the lid and avoiding convection within the wells 24 which sometimes cause an uneven distribution of cells throughout a well 24. The temperature control circuit 791 is able to be electrically coupled to the impedance PCB 708, the sensor interconnect circuit 706, the substrate 12 and/or another electrical circuit for sending the electrical signals to control the temperature of the lid 26 and/or wells 24.

In some embodiments, the lid 26 is able to not have a lip such that the sides of the wells 24 are not occluded in any way by the lid 26. In some embodiments, the lid 26 is made of transparent plastic (e.g. zeonex) having low birefringeance, low loss, and repeatable properties. In some embodiments, the lid 26 is able to include one or more lenses or other optical components as described herein. In some embodiments, the lid 26 includes one or more grooves for gluing the lid 26 to the wells 24. In some embodiments, the bottom side of the lid 26 (with the rim guides 793) is able to be coated with a hydrophobic or hydrophilic layer to control condensation and its effects on image capture. In some embodiments, the portion of the bottom of the lid 26 within and/or including the rim guides 793 is able to be arched or concave (or non-flat) such that the perimeter of the guides 793 is lower than the bottom of the lid 26 within the guides 793. This provides the benefit of encouraging any condensation droplets to form or move to the perimeter of the cells 24 rather than the central portion where they can affect image capture. In some embodiments, the inside surface of the walls of the cells 24 (and/or the portion of the bottom of the lid 26 above the wells 24) is able to include one or more ridges that guide condensation along the ridge back into the main body of fluid within the well 26. This provides the advantage of reducing both occlusion and analyte concentration changes caused by condensation.

In some embodiments, the lid 26 comprises one or more light emitting diodes (LEDs) for directing light at the wells 24. In such embodiments, the LEDs are able to be positioned on a top of the lid 26, within the lid 26 or protruding through the lid 26 such that they are able to direct light into each of the wells 26. Such embodiments have the benefit of enabling illumination of each well 24 to be controlled independently, enabling the light to be turned off or adjusted when probe 704 measurements are being made, enabling the light to be flashed to “freeze motion,” enabling the light to double flash to measure particle velocity, enables light amplitude adjustment for each well 24 on a per image captured basis to improve image uniformity, enables brightness to be controlled by selecting smaller portions of each subfield (e.g. instead of normalizing the average brightness of a subfield, one can select areas that do not contain electrodes), and enables subfields near walls to be less effected by reflection, refraction from the walls.

FIG. 7V illustrates a method of implementing the SPA 702 according to some embodiments. As shown in FIG. 7V, the SPA 702 is moved to the open position at the step 701. The electronic interconnects substrate 12 is positioned within the substrate recess 748 at the step 703. The slide 22 is positioned on the electronics interconnects substrate 12 above the imaging holes 742 and/or electrodes 16 at the step 705. The lid 26 is positioned on the slide 22 at the step 707. In some embodiments, positioning the lid 26 on the slide 22 comprises sliding the top of each of the wells 24 into one of the well rim guides 793. In some embodiments, positioning the lid 26 on the slide 22 comprises gluing the lid 26 on the slide with an adhesive (e.g. silicone glue, epoxy). The SPA 702 is moved to the closed position at the step 709. In some embodiments, moving the SPA 702 to the closed position comprises the slide 22 moving through the slide window 760 of the slide holder 716 such that the prongs 726 of the guide plates 718a, 718b contact the outward facing walls of the wells 24 of the slide 22 and thereby guide the slide/wells into the correct position within the SPA 702 with each well 24 aligned with one of the imaging holes 742. In some embodiments, where in the outer walls of the wells 24 are inwardly angled such that the contact between the guides 718a, 718b and the outer walls causes the wells/slide to move toward the correct position. In some embodiments, moving the SPA 702 to the closed position comprises detachably coupling the lock bolt 720 to a lock bolt aperture 754 and/or compressing one or more of the push rods 720. In some embodiments, moving the SPA 702 to the closed position comprises detachably electrically coupling the impedance PCB 708 to the electrodes 16/contact pads 18 of the substrate 12 for controlling and/or detecting impedance measurements of the samples within the wells 24.

One or more optical probes 704 are coupled through probe passages 766 of the slide holder of the SPA 702 (and/or within the probe recess 762) such that they point toward indicators on walls of the wells 24 at the step 711. In some embodiments, coupling the probes 704 comprises extending a first portion of the probe 704 through the first larger diameter portion of the passage 766 and a second narrower portion of the probe 704 through a smaller diameter portion of the passage 766. In some embodiments, the narrower portion is one or more central optical fibers of the probe 704. One or more sensors 710 are positioned partially within one or more of the wells 24 at the step 713. In some embodiments, positioning the sensors 710 within the wells 24 comprises inserting the sensors 710 through one or more holes 798, 799 of the lid 26 such that they protrude down a side or non-central portion of the well 24. In some embodiments, positioning the sensors 710 within the wells 24 comprises gluing each of the sensors 710 to one of the sensor supports 795 (and/or sealing any space between the sensor 710 and the holes 798, 799). In some embodiments, the method further comprises inserting a syringe into one or more of the angled holes 797 of the lid 26 for adding or retrieving culture media from one or more of the wells 24. In some embodiments, the method further comprises irradiating the slide 22, the lid 26 and the sensors 710 with gamma radiation for sterilization. The sensor interconnect circuit 706 is coupled to the one or more sensors 710 at the step 715.

In some embodiments, the method further comprises detecting polydata from one or more of the samples within the wells 24 using the sensors 710, optical probes 704 (and indicators within wells 24), the imaging unit 36 capturing images through the imaging holes and the bottom of the wells 24 and/or impedance data from the substrate 12 and impedance PCB 708. For example, multiple different types of chemistries (e.g. O2, CO2, pH, glucose, temperature, image capture, impedance, secretome and/or the other types of sensors described herein) are able to be sensed and recorded in parallel from each of the samples within the wells 24 using the system 10. In particular, the same group of chemistries is able to be sensed and recorded in parallel for each well 24 in the slide, or different groups of chemistries are able to be sensed and recorded in parallel for different subsets of the wells 24 of the slide 22. Further, in some embodiments the method further comprises turning off one or more of the other sensors when using a particular sensor to reduce noise, interference or other undesired effects the other sensors have on the desired sensor. For example, the impedance sensors (e.g. electrodes 16) are able to be turned off when sensing glucose levels. In some embodiments, a multiplexer is able to be used so that only one electrical sensor 710, 16 is galvanically connected to a well 24 for taking a measurement at any one time. As another example, the illumination from the image capture 36 (and/or an LED of the lid 26) is able to be turned off while reading pH, O2 or other light sensor/guide/chemical dot based measurements (and vice versa). Indeed, any combination of the sensors are able to be turned off and on as desired such that only a desired subset is actively detecting measurements during a desired window of time.

Thus, the SPA 702 provides the advantage of allowing for repeatable positioning of optical probes relative to the wells 24/slide 22 for pH, O2 and/or other probe based chemistry measurements. In particular, when in the closed position, the guide plates in combination with the slide holder and the angled sides of the wells 24 enables the slide 22/wells 24 to consistently positioned as close as possible to and with a repeatable alignment with the optical probes (e.g. the proximate ends of the optical fibers thereof) as coupled through the fiber passages of the slide holder. Indeed, this is able to overcome molding tolerances of the slides 22/wells 24 thereby producing better optical path coupling and reduced wavelength tilting/tilt variations between the optical probes 704 and the sensors (e.g. sensor dots) on the wells 24 (and/or inserts within the wells 24).

### Slide and Well Design Variants

Variants of well 24 construction and coupling of an optical assembly (e.g. optical probe 704 and/or imaging unit 36) to the wells 24 are described herein. The objectives for these variants are to generate an assembly for optical sensing of the chemistry in the well 24, lower the cost of the consumable and shift complexity to the non-consumable portion of the instrumentation, increase the density of chemical sensing from the side-wall of the well 24, implement a “non-consumable” portion of the system that enables the above objectives, and avoid damaging the optical coating of the optical probes (e.g. optical probe 704 and/or imaging unit 36).

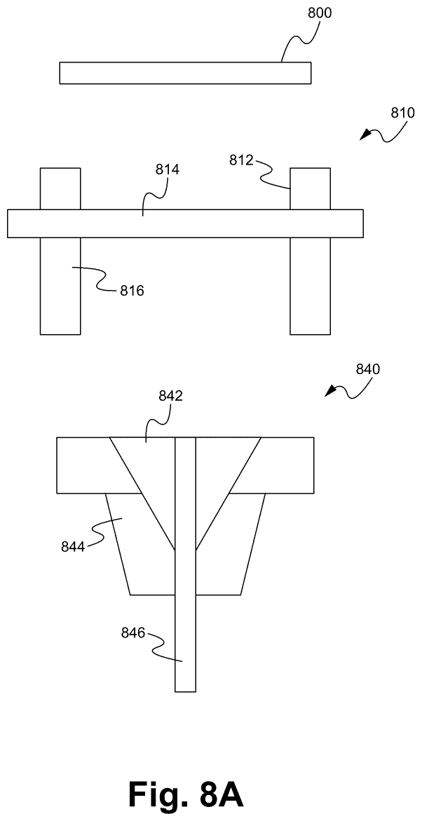

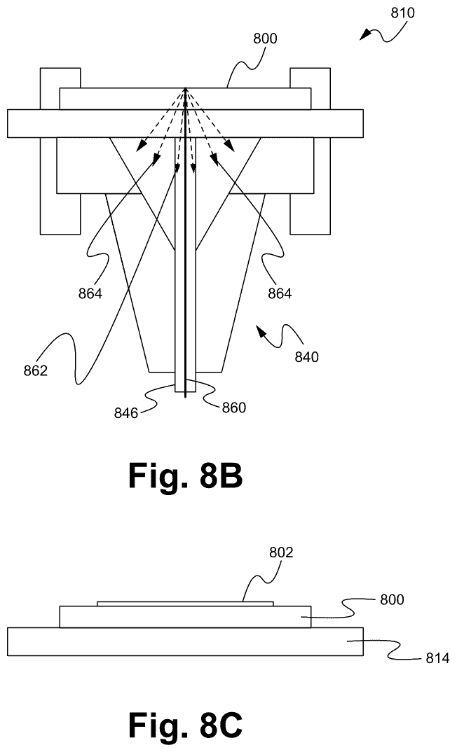

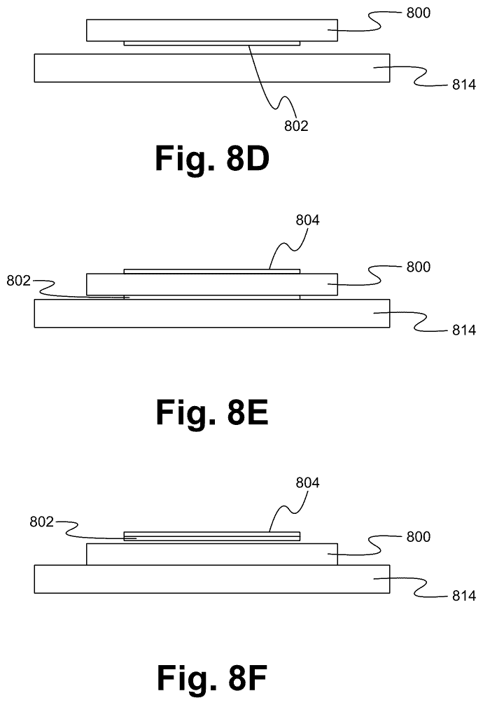

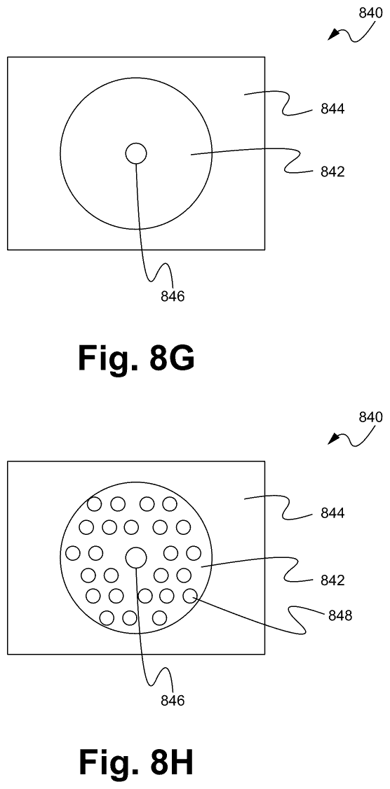

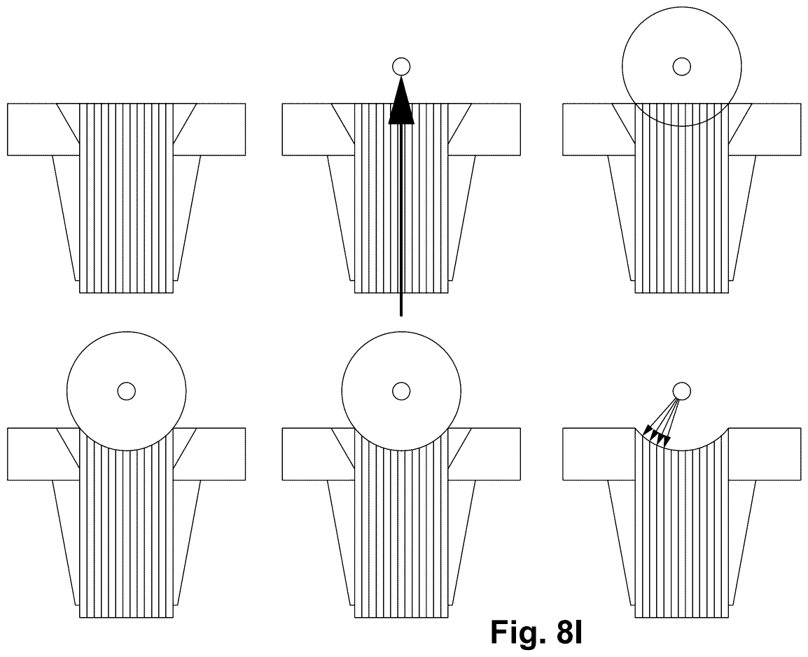

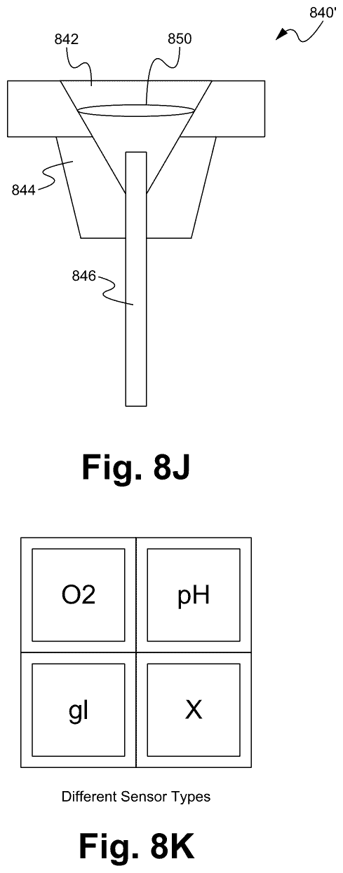

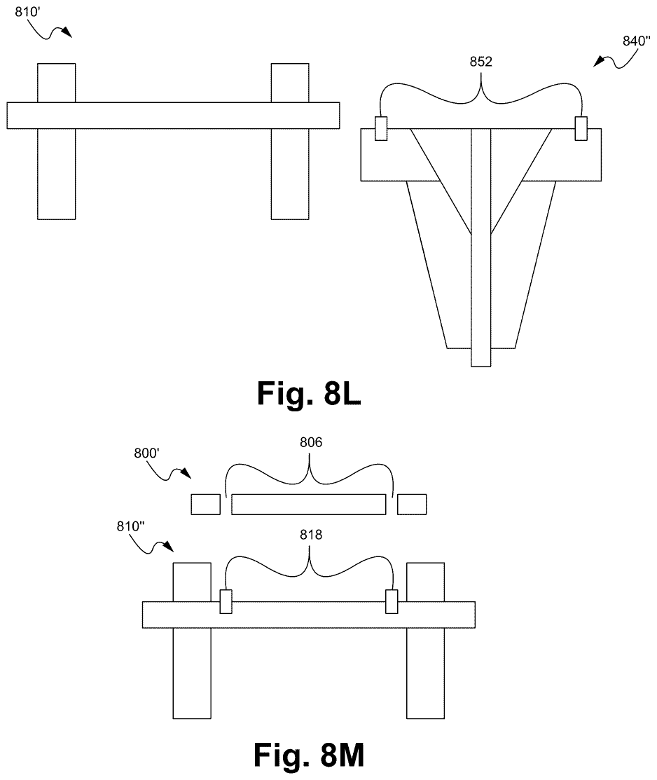

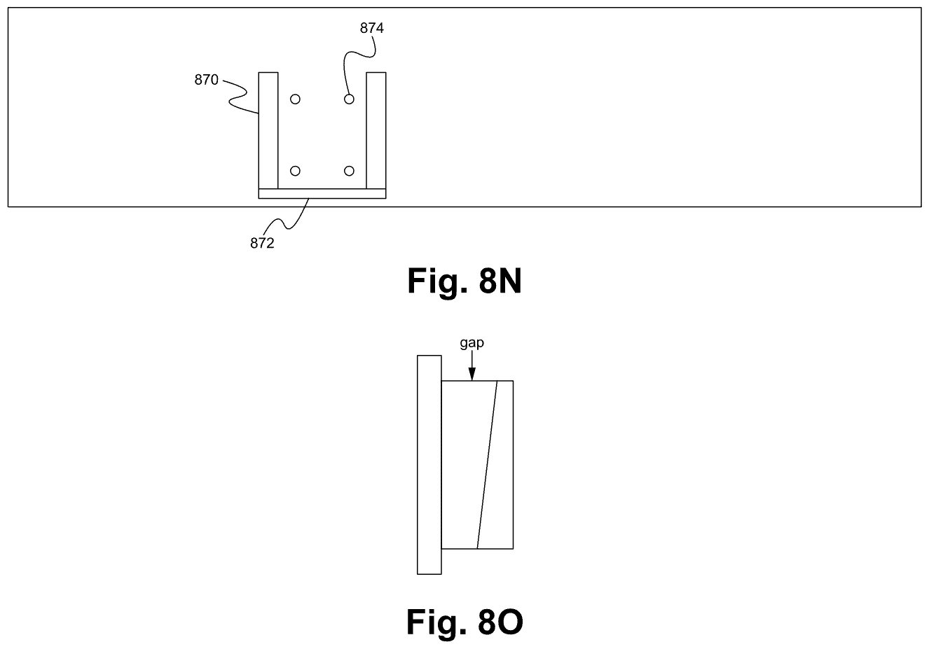

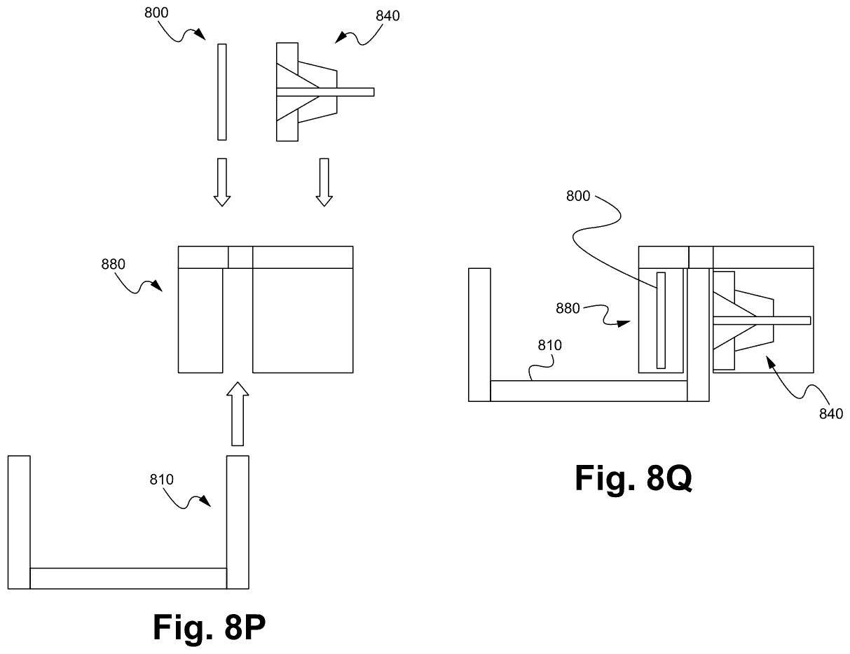

FIG. 8A illustrates a high-level view of the variant of well construction according to some embodiments. The features of the wells, slides, light guides, sensors, inserts and/or imaging units described with respect to FIGS. 8A-Q are able to be incorporated into the wells, slides, light guides, sensors, inserts and/or imaging units of the other figures described herein. A transparent slide 800 (e.g., plastic or glass) includes an optically active sensor (e.g. chemical sensor dot) which fluoresces and/or changes color based on chemistry of the environment of a well. A slide has chemical sensors on it. The slide is optically transparent and can be placed inside the well. The chemically active portion is on the upper side closest to the sample so that when it is slid into a modified well wall 810 the chemistry is not scraped off.

The modified well wall 810 has three distinct pieces: the upper piece 812 is a clamp on the inside of the well wall which is a holder for the slide 800. The middle portion 814 is the well wall. The bottom piece 816 is a mechanical structure/clamp on the outside of the well wall that is a holder for an optical assembly 840.

The well wall 810 is constructed in a manner to hold the slide 800 in close proximity to the inside of the well wall 810. The well wall 810 also has a mechanism to hold an optical assembly 840 in close proximity to the outside of the well wall 810 and also in a manner where the slide 800 and the optical assembly 840 are properly aligned for optimal performance and signal sensitivity.

The purpose of the construction in this fashion is that one is able to customize a slide 800 and make it disposable. The optical assembly 840 is a fixed port of the system that can be reused and is not intended to be disposable. The optical assembly 840 has the function of being mechanically coupled to the well wall. The portion 844 provides mechanical alignment. The optical fiber 846 reaches all the way to the well wall so that an excitation ray can be sent to the slide 800, and the resulting fluorescence can be received by the fiber. The triangular portion 842 has mechanical characteristics. Additional figures show potential optical characteristics and how the fiber and holder can be modified in order to collect more light. The rationale behind this construction is to allow for alignment between the slide 800 and the optical assembly accomplished through self-alignment with the wall using the modified well wall 810. In some embodiments, multiple sensors are utilized. The other rationale is that the optical fiber piece is expensive and is preferably reused, but the sensor piece which is exposed to the samples and chemically active is consumable.

FIG. 8B illustrates a combination of a slide, a well wall and an optical assembly according to some embodiments. Incident light 860 comes from an instrument through the fiber 846 (FIG. 8A). Collected fluorescent light 862 comes from the coating and makes its way back through the fiber and into the instrument for measurement. Uncollected fluorescent light 864 comes from the coating but is reflected onto the triangular portion 842 (FIG. 8A) and is therefore not included in the measurement. Some of the incident light 860 will reflect back as well. The intensity of the fluorescent light (e.g., collected) is dependent on the fluorescence changes resulting from the chemical environment and is also proportional to the intensity of the incident light 860.

FIG. 8C illustrates a combination of a slide and a well wall according to some embodiments. The slide 800 is shown with a chemical sensor element 802. The chemical sensor element 802 faces the inside of the well. The slide 800 is optically transparent. The chemical sensor element 802 is the layer that fluoresces with a chemical. The side wall of well 814 (also referred to as the middle portion) is also shown.

FIG. 8D illustrates an alternate construction of a slide and a well wall according to some embodiments. A chemical sensor element 802 is sandwiched between the slide 800 and the side of the well wall 814. Gaps between the sensor and the side-wall or holes in the slide 800 allow for enough diffusion to occur from the inside of the well to the chemical sensor element 802 and allow the chemical sensor element 802 to be closer to the fiber 846 (FIG. 8A). An indentation is intended to prevent the fluorescent layer from being scratched when inserted in to the holder.

FIG. 8E illustrates an alternate construction of a slide and a well wall according to some embodiments. A chemical sensor element 802 is sandwiched between the slide 800 and the side of the well wall 814. Gaps between the sensor and the side-wall or holes in the slide 800 allow for enough diffusion to occur from the inside of the well to the chemical sensor element 802. A reflective layer 804 reflects back the fluorescence from the chemical sensor element 802 toward the fiber 846 (FIG. 8A). This construction allows the chemical sensing element 802 to be closer to the fiber 846 (FIG. 8A). An indentation is intended to prevent the chemical sensor element 802 from being scratched when inserted. The reflective layer 804 reflects back the fluorescence from the chemical sensor element 802 that would normally escape into the well back toward the fiber 846 (FIG. 8A).

FIG. 8F illustrates an alternate construction of a slide and a well wall according to some embodiments. A chemical sensor element 802 is exposed to the inside of the well 814. A reflective coat 804 is placed on the chemical sensor element 802. This reflective layer has holes in it to allow enough chemical diffusion to occur from the inside of the well to the chemical sensor element 802. The reflective layer 804 reflects back the fluorescence from the chemical sensor element 802 that would normally escape into the well back toward the fiber.

FIG. 8G illustrates a front view of an optical assembly according to some embodiments. The triangular portion 842 is the optically transparent material which surrounds fiber. The circular shape can also be square or hexagonal or other shapes. The portion 844 is plastic or another material which provides physical robustness for handling the fiber 846 and also to mate to the clamp on the exterior wall of the well.

FIG. 8H illustrates a front view of an optical assembly with multiple fibers according to some embodiments. The optical assembly can have two or more optical fibers 846 and 848. The extra fibers 848 can be used for calibration purposes or for additional flexibility in sensing. The extra fibers 848 can also be used so that one fiber is optimized for providing a stimulus to the chemical sensor element, while the other fibers are optimized to receive the maximum response from the chemical sensor element.

The optical assembly 840 mates to the outside of the well wall. The optical assembly 840 allows for “attachment” to the well in a manner that is secure, and well aligned. This optical assembly 840 includes multiple components: a mechanical component to hold the various pieces together, an optical fiber or multiple optical fibers that are aligned to direct light at the slide 800 containing the chemical sensors and receive the fluorescent response from the chemical sensors. In an embodiment of a single fiber, it acts as providing the “stimulus” light to the chemical sensors as well as receiving the “response” light from the chemical sensors. In an embodiment of multiple fibers, one of the fiber elements can be optimized for delivering the “stimulus” light to the chemical sensors, while the other fibers can be optimized for receiving the “response” light from the chemical sensors. In some embodiments, the diameter of each fiber type can be different (for optimization purposes), and the optical properties of the different fiber types can be different (for optimization purposes). A mechanical component securely connects the optical assembly to the well side-wall. A separate fiber or bundle of fibers are used for collecting the (emitted) fluorescence signal. The bundle of fibers can be a large bundle to collect more emitted signal.

If the excitation fiber is located in the center of the emission collection bundle, the fibers can all be pointed in the same direction and the sensitivity to gap variability is reduced. All the receiving fibers are pointed toward the highest fluorescence emission point on the chemical sensor which is the intersection point of the excitation point of the chemical sensor.

In some embodiments, a composite assembly is implemented with curved fibers for higher effective NA collection. The fiber strands are aligned to point toward the point of emission from the chemical sensor element.

FIG. 8I illustrates a diagram of a composite assembly with parallel fibers and a semi-circular phase for higher effective NA collection according to some embodiments. The diagram shows an easier way to construct this composite assembly by polishing the fiber ends. In a first step, parallel fibers are generated in a bundle. In a second step, the location is determined from which the chemical sensor will fluoresce. In a third step, concentric circles are constructed around this point of emission. In a fourth step, the fibers and the assembly are polished to match the larger concentric circle. In a fifth step, the fibers and the assembly are polished to match this larger concentric circle. In a sixth step, the emission source radiates in the fashion shown in the last image. The fibers can have different endings. They can be budding to the well wall or further down.

In some embodiments, all fibers are parallel to each other and coplanar and touching the well wall. In another implementation, all fibers are parallel to each other but the surface of each individual fiber is pointing towards the source of emission.

In some embodiments, all fibers are pointing towards the source of emission. If the emission fiber or bundle of fibers is separate, the fibers are pointed at slight angles so their beams intersect at the sensor. This allows the use of more readily available fibers. This also permits the use of one emission fiber.

For those skilled in the art, it should be clear that there are many variables such as distance from the source of emission or combinations that are possible.

If the detector is a pixelated avalanche detector (e.g., Hamamatsu MPPC), the emission bundle can be “jumbled” to illuminate the detector more uniformly. It can also be square at the detector end.

Instead of an emission bundle, a single molded plastic light guide can be used to collect and transfer the light to the detector. It can be separate or co-molded with the excitation fiber.

The excitation fiber can be replaced by a laser diode or LED emitter located near the sensor, and co-molded with a light guide to provide an inexpensive, single piece solution. Excitation and/or emission filters can be coated onto the emitter and/or light guide, respectively.

FIG. 8J illustrates a variant of an optical assembly according to some embodiments. A lens 850 is inserted in the path of the incident and fluorescent light. In this implementation, the lens 850 is designed to collect more of the fluorescent light and focus it on the fiber, thereby enhancing the receive signal. The excitation signal from the fiber is generally unchanged. A lens 850 can be arranged to collect more of the emitted fluorescence and “routes” it to the optical fiber 846.

The optical assembly is able to be made and used in multiples (e.g., four). A slide is able to be made and used in multiples (e.g., four). Each slide is able to include a different chemical sensor.

The optical assembly or the well side-wall can have optical elements (such as lenses) that “gather” light that is emitted from the chemical sensor and focus this light onto the tip of the receiving fiber. This allows for a larger “received” signal to be gathered because the emission of the chemical sensor is “in all directions.” Using a lens allows us to collect “light rays” that would have otherwise missed the “entry point” for the fiber. It can be seen that this mechanism can increase the collected signal. It can also be combined with reflective surfaces (within the well that allow for the diffusion of chemicals to the sensors) that collect and redirect the emission from the chemical sensor into the well, back into the fiber.

FIG. 8K illustrates a multi-sensor element according to some embodiments. The multi-sensor element is able to include identical sensors or different sensors. For example, one sensor is an O2 sensor, another is a pH sensor, a third is a glucose sensor and finally an inert sensor (or another sensor). In another example, there are three sensors that are O2, and one that is inert by placing a barrier so that the chemicals are not activated and that can be used for calibration.

In some embodiments, each of the sensors or sensor types has its own optical fiber. This leads to a multiplicity of optical fibers coming from the well structure. In some embodiments, multiple fibers have been “brought together” in a tightly packed form factor. This allows these fibers to then be “bundled” together in a much more compact form because they all emanate from one assembly.

The other end of the optical assembly can have a similar construction where the individual fibers are “routed” to the appropriate instrumentation for measurement. Alternatively, because the fibers will be arranged in a pre-determined form, the optical sources and sensors in the instrument can benefit from a significant simplification. The simplification can be in the form of “multiplexing” between different fibers in a mechanical manner or through photonics mechanisms (used for multiplexing or routing optical signals) or through MEMS micro-mirrors. This allows each sensor/fiber to be used in a time multiplexed manner in order to reduce the amount of electronics and optical components in the measurement instrument.

FIG. 8L illustrates a diagram of an optical assembly and a well side-wall using pin/protrusions according to some embodiments. A mechanism is able to be used to align the optical assembly 840″ to the well side-wall 810′ using pins/protrusions 852 that will provide lateral mechanical alignment. Notches in the wall of the well mate to the optical assembly 840″. Four of these notches can fix the optical assembly 840″ to the well side-wall 810′. Retractable pogo pins are able to protrude from the optical assembly 840″. As the density of the sensor elements increases, the alignment between the sensor element to the well-wall and the alignment of the optical assembly become more and more important. This mechanism locks it in so that movement and vibrations will not affect the alignment to the well wall.

A mechanism is able to be used to align the optical assembly to the well side-wall using pins/protrusions that will provide mechanical alignment. As the density of the sensor elements increases, the alignment between the sensor element to the well-wall and the alignment of the optical assembly to the well wall become more and more important. FIG. 8L shows the alignment between the well wall and the optical assembly. The mechanism to align the optical assembly to the well side-wall using pins/protrusions that will provide mechanical alignment is shown.

FIG. 8M illustrates a mechanism to align the slide/sensor element to the well side-wall using pins/protrusions provides lateral mechanical alignment according to some embodiments. The notches 806 in the slide 800′ mate to the well wall 810″. Nubs 818 and notches (e.g., four) can affix the slide 800′ to the well wall 810″.

FIG. 8N illustrates a side view of well wall exterior according to some embodiments. Clamp 870 is on the exterior wall of the wall. Notches 874 on the well wall enable mating with an optical assembly. A stopper 872 at the bottom of the side-clamps is outside the well.

FIG. 8O illustrates a side view of a gap between the well wall and an optical assembly according to some embodiments. In some embodiments, the gap between a bottom piece 816 and a middle piece of the well wall 814 is constant from the top to the bottom of the bottom piece 816. In order to make the optical assembly 840 and the bottom piece 816 mate together properly, the operator would have to expend a lot of care in order to make sure that the two are inserted into each other with precision and care. In order to simplify the task of the operator, the construction of the bottom piece 816 can be modified such that the gap between the bottom piece 816 and the middle piece of the well wall 814 reduces the top portion and the bottom portion. The optical assembly 840 would have a corresponding change in thickness (top is thicker than the bottom at the point of mating with the bottom piece 816).

Mechanisms that allow the easy insertion of the sensor slide and the optical assembly into their respective holders on the side-wall of the well (inside the well and outside the well) are shown. In some embodiments, one can use tapering in order to insert these objects into the side-wall. Different types of tapering are possible. One type of tapering is done on the areal aspect where if one views the slide or the optical assembly at a perpendicular angle to the side-wall, one would see a trapezoidal shape. This trapezoidal shape allows the object (slide or optical assembly) to be initially inserted first using the narrow side of the object into the wide side of the holder. As the object is pushed further in, the holder also narrows-such that once fully in, the object and the holder have a tight fit. This allows for an easy insertion and placement of the objects into their respective holders. The other type of tapering is observed when one views the objects in the plane of the side-wall. Again, having the entry point of the object being thinner that the entry point of the holder allows for easy insertion. The side view for both the object and the holder will appear as trapezoids.

In some embodiments, a scratch protection mechanism is implemented. An indent is generated on an outer wall of the well. The scratch protection mechanism protects the optical assembly's surface from being scratched when it is slid into place. A soft optically transparent layer/coating is generated to protect the optical assembly from being scratched. Properties include: optically transparent, mechanically soft, plus other optical properties. The layer/coating is able to be replaced after every use. In some embodiments, the film can be peeled off, or it can be a film that is applied chemically and removed chemically, or it can be a form of lubricant that can be dissolved away. All of these operations are designed to be prevent damage to the non-consumable part of the optical assembly. The wall of the well that is in contact with optical assembly is optimized for its optical properties, and minimizing damage to the surface of the optical assembly.

FIG. 8P illustrates a side view of a diagram of a clip-on attachment according to some embodiments. The figure shows how sensing elements fit on the clip-on insert 880 which fits on the well wall. A slide 800, a well wall 810 and an optical sensor 840 are shown.

FIG. 8Q illustrates a side view of a diagram of a clip-on attachment according to some embodiments. showing how the clip-on insert 880 fits on the well wall 810. The Figure shows how the full assembly fits together. This design has the benefit of flexibility because people do not have to buy special wells. These clip-on inserts can be used on any well and are sterilizable. All prior implementations up until this point have been “built-into” the well structure.

This allows the use of a lid for the well structure to protect it from external contaminants during the incubation process. An alternative is envisioned where a “standard” well structure is used in conjunction with an add-on apparatus. One implementation is to “clip on” the optical assembly onto the top of the well wall. The clip-like structure is able to fit over the side wall of the well.

Another implementation is to connect the optical assembly through a screw structure and sealing O-ring through a hole in the well wall.

A sensor assembly can be calibrated by exposing the sensors in the well to predetermined concentrations of different chemicals.

The cross-talk between different sensor types can also be measured and calibrated. For example, it is possible that a sensor that is used for glucose measurement might have a dependency on pH or on O2 levels. If so, then a “calibration” curve can be generated to compensate for any such dependency or cross-talk.

Prior invention disclosures have described how a well can be filled or emptied using fluidic channels in the side-walls or elsewhere. Even if such fluidic channels are not present, a calibration can be done by other means of filling or emptying the wells.

### Polybiosensing-Imaging System having Collapsible Wall

A sensing structure for wells 24 is described herein. Nominal dimensions are in the 20 um-100 um range (e.g. 20×20×20 um cube sized well) for the well size or for the cell clusters, although other range sizes are possible. These ideas can be applied to monitoring the following categories: a single cell, a cell cluster, an organoid, and a spheroid. The goal is to measure the relevant chemistry in the well 24 with the size constraints that have been stated above. The chemicals of interest are able to be pH, glucose, O2, CO2, or any other chemistry of interest which can be measured. This could be done through optical means-where special chemical sensors fluoresce once exposed to each of the above chemicals (e.g. chemical sensor dots). A fiber optic cable provides the stimulus, and the same or other bundles of cable pick up the fluorescent response (e.g. optical probes 704). In some embodiments, the fiber optic cables (e.g. probes 704) are on the outside of the well wall, and the chemical sensors are on the inside of the well wall. As the well size decreases (e.g., Genius Wells™ gets miniaturized), alternative arrangements can be advantageous.

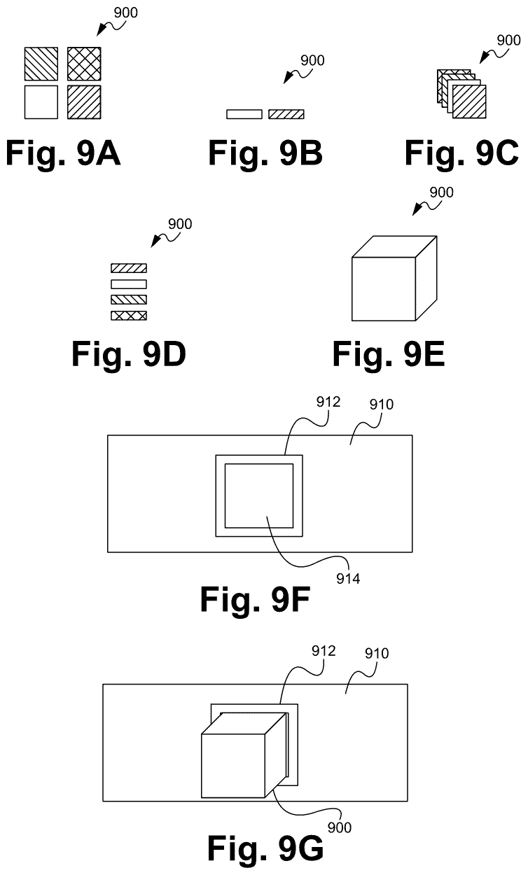

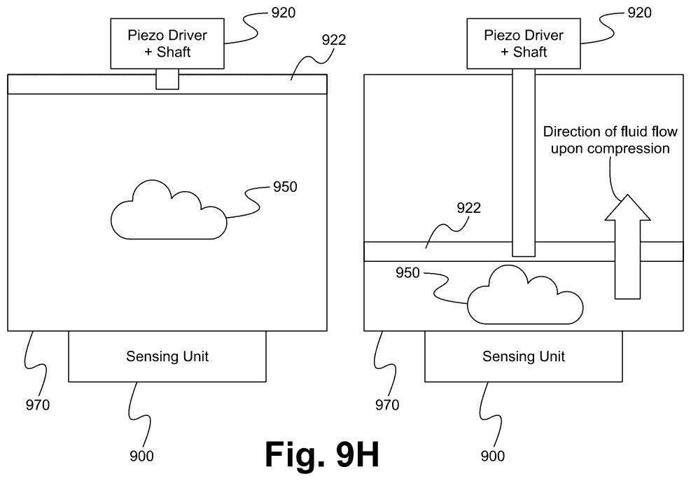

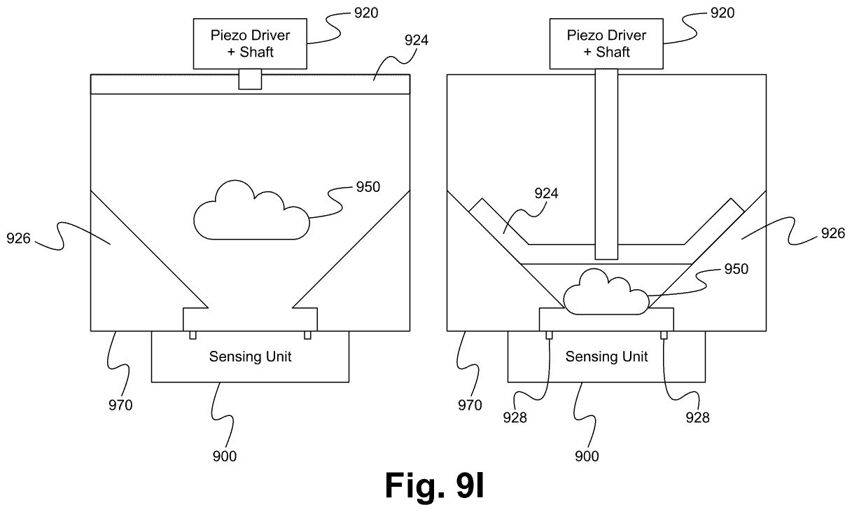

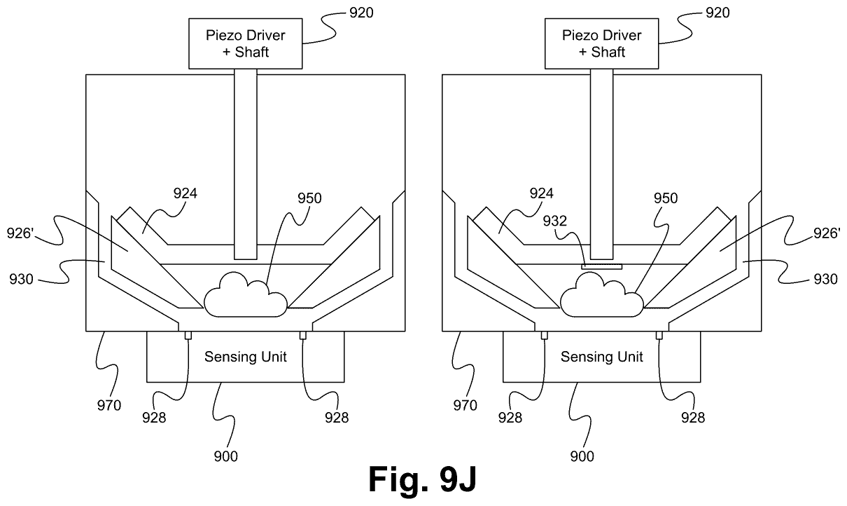

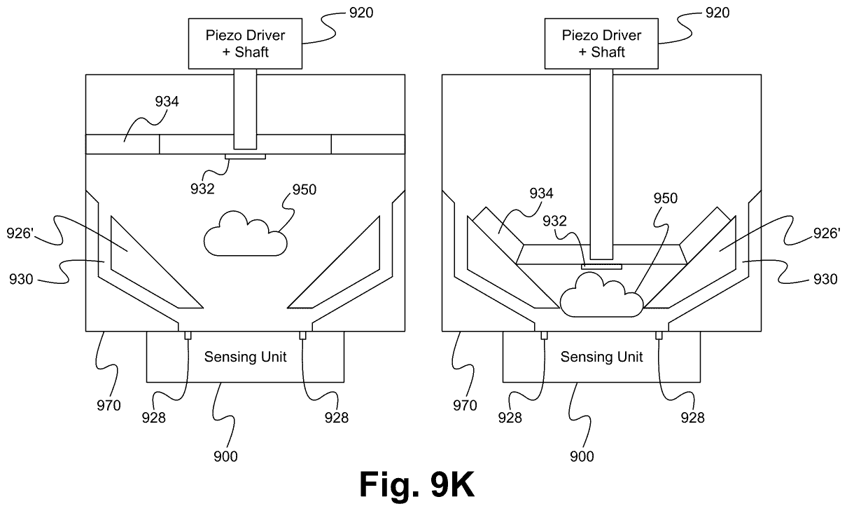

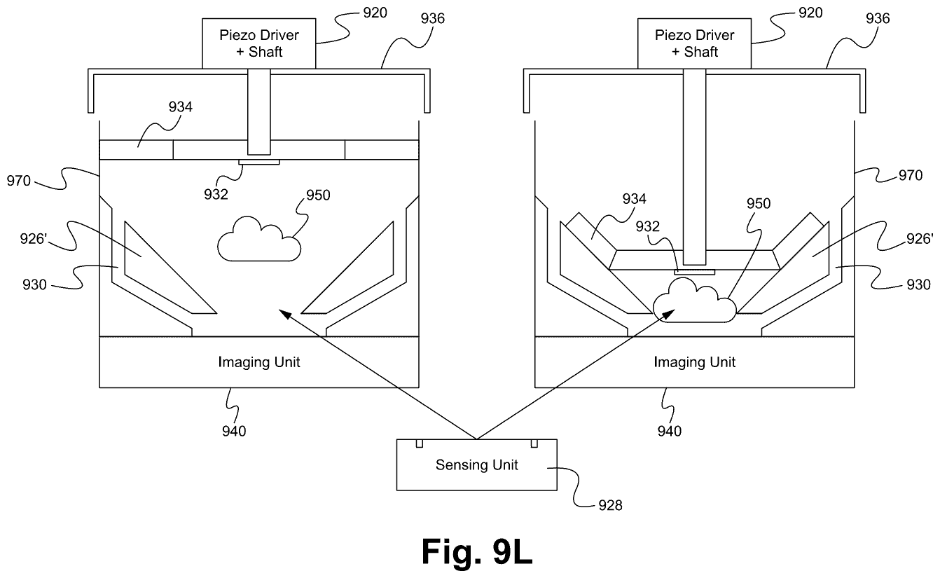

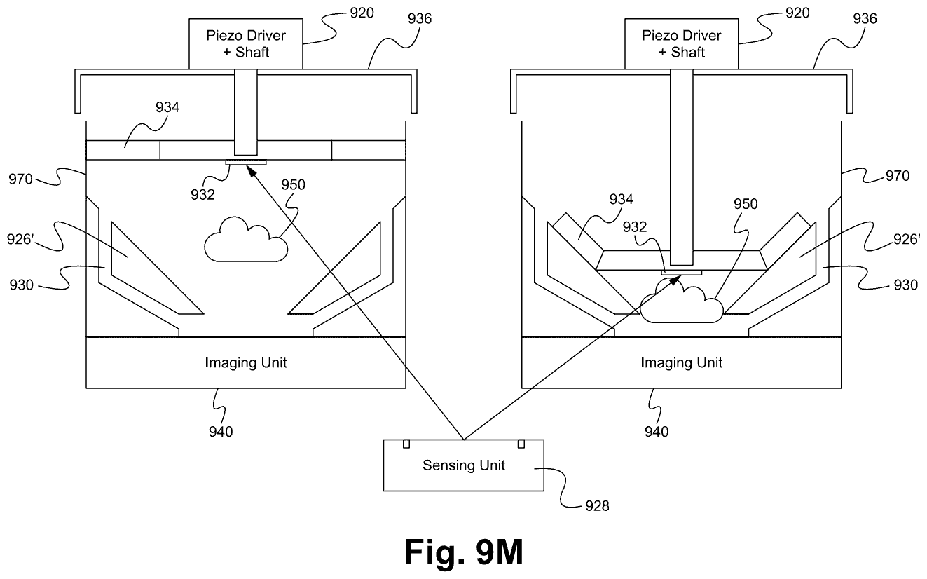

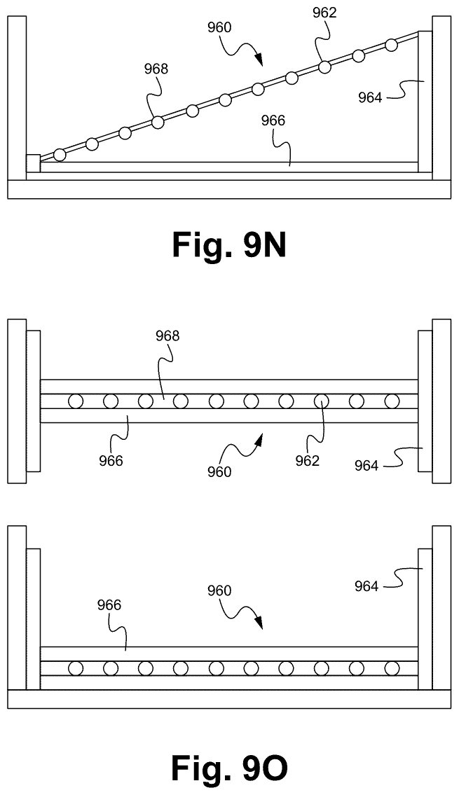

The fiber optic and chemical sensors can be miniaturized to a certain level which is dictated by the available technology. As the well 24 size decreases, the thickness of the well wall can become a significant factor in the ability to accurately measure the chemistry in the well. 24 The sensor size decreases, the width of the fiber optic cable may decrease, and one should avoid hitting the cell cluster with the excitation laser pulse from the optical fibers. If one uses other sensor types (ISFETs or other solid-state sensors), the size of these sensors should be kept small. Given the desire to obtain optical images from the bottom of the well 24, there are a limited number of places where these sensors can be inserted: the sensor group can be inserted from above into the well, the sensor group can be placed on the well 24 using a clip-on mechanism, and the sensor group can be inserted into the well 24 through a hole in the well wall. Incorporating sensors through the well wall: the solid state sensors can be arranged on a plane which minimizes the depth of the assembly, or the solid state sensors can be arranged in a stacked fashion where there is a gap between the sensors to allow the medium from the well to flow and to provide a pathway for the chemicals to diffuse to the sensors, which minimizes the areal footprint of the assembly, but increases the depth of the assembly. A combination of the above can also be implemented. In all cases, the chemical sensing surface is exposed to the media, but the other surfaces are coated with material that is biocompatible with the cell clusters being tested. The features of the wells, slides, light guides, sensors, inserts and/or imaging units described with respect to FIGS. 9A-O are able to be incorporated into the wells, slides, light guides, sensors, inserts and/or imaging units of the other figures described herein.

FIG. 9A illustrates a top view of an assembly with solid state sensors arranged on a plane according to some embodiments. Arranging the solid state sensors 900 on a plane minimizes the depth of the assembly.

FIG. 9B illustrates a side view of an assembly with solid state sensors arranged on a plane according to some embodiments.

FIG. 9C illustrates a top view of an assembly with solid state sensors arranged in a stacked fashion according to some embodiments. There is a gap between the solid state sensors 900 to allow the medium from the well to flow and to provide a pathway for the chemicals to diffuse to the sensors, which minimizes the areal footprint of the assembly, but increases the depth of the assembly.

FIG. 9D illustrates a side view of an assembly with solid state sensors arranged in a stacked fashion according to some embodiments.

FIG. 9E illustrates a view of a generalized shape of an assembly with solid state sensors according to some embodiments. Although an exemplary shape is shown, any shape is able to be implemented.

FIG. 9F illustrates a diagram of an opening within a well wall according to some embodiments. The well wall 910 includes an opening 914 (e.g., hole) with a gasket 912. The opening 914 can be in the form of a hole or a cut-out in the wall 910 which extends to the top of the well wall 910.

FIG. 9G illustrates a diagram of a sensor assembly within an opening within a well wall according to some embodiments. The sensor assembly 900 is coupled to the opening 914 in the well wall 910. The gasket 912 between the wall 910 and the sensor assembly 900 generates a seal against liquid or gas exchange between the inside of the well and the outside of the well. In some embodiments, the sensor assembly 900, gasket 912 and well are clamped or glued together to achieve the above. The rear side of the sensor assembly 900 is also sealed such that the inside of the well is connected to the sensors inside the sensor assembly 900. The well is extended by a finite volume that houses the various sensors. This allows incorporation of an array of sensors for various measurements and allows the sensors to get close to the cell cluster. In cases where the well size is small and the sensor assembly 900 occupies a volume that is substantial, this technique can be used. The sensor assembly 900 can be re-used with the appropriate amount of cleansing or it can be disposable.

Another method for sensing in small wells is to extract selected cells through a micro-fluidic channel to isolate them from the remaining population. These cells can be placed in a “miniaturized genius WellsTM” such as a micro-well, whose volume is very small.

Another method for sensing in small wells is to generate a collapsible well whose volume can be adjusted electronically. This moves away from the paradigm of a fixed well size for making measurements. In this method, it is assumed that there is only one cell cluster within the well. This assumption is made for the purpose of simplicity. In a further modification of this method, this assumption will be removed. With this collapsible well, the following applies: preserve the imaging capability from the bottom, and preserve the impedance measurement capability where the electrodes are on the bottom of the well. Additionally, because the well is collapsible, impedance measurement capability can be added to the sensor array that resides on the side-wall of the well. This can be in any of the implementations that have been described herein (optical measurement methods through the well wall or a measurement unit that is inserted through a hole in the well wall so that the measurement unit is in direct contact with the media).

There are multiple ways to implement the collapsible well.

FIG. 9H illustrates top views of a collapsible well according to some embodiments. The collapsible well includes a rectilinear well structure 970. One or more sensor units 900 are arranged proximate to one of the well walls. The well wall area is made as small as possible to allow the sensors to make measurements without compromising measurement accuracy. The moveable well wall 922 that is opposite the sensor arrangement 900 is electronically moveable as shown. A piezo driver and shaft 920 are coupled to the well through a hole 914 (FIG. 9F) and a gasket 912 (FIG. 9F) that allows the well to be sealed. The Figure shows the piezo driver and shaft 920 in a retracted position and in an extended position, where the moveable well wall 922 reduces the amount of open space the cell 950 is in.

The piezo element 920 (also referred to as piezo driver and shaft) is controlled electronically while the cell 950 is monitored optically by the camera system. The piezo element 920 is coupled to a moveable wall 922 which moves to reduce the volume of media that is proximate to the cell 950 and the sensors 900. The moveable wall 922 does not have to be completely sealing on its sides. This will allow media to flow from one side of the well to the other. The moveable wall 922 can be rigid or semi-rigid. The moveable wall 922 may also be perforated to allow easier fluid flow.

FIG. 9I illustrates top views of a collapsible well according to some embodiments. A moveable wall 924 is made of a flexible material that will bend to conform to the contours of the well walls. The collapsible well includes constricting well walls 926 to further enable reducing volume. By doing so, the moveable wall 924 will allow the minimization of the volume proximate to the cell 950 and the sensing unit 900. If the aperture 928 of the sensing unit 900 is larger than the smallest dimension of constricting walls 926, then a gap is provided so that the sensing unit 900 can still be in contact with the fluid from the media.

The constricting walls 926 can be vertical—thereby constricting in the x-dimension. The constricting walls 926 can also constrict in the x and z dimensions—in which case, they will be tapered in the z dimension. For example, z1 is near the bottom of the well, z2 is higher than z1, z3 is higher than z2 and so on. This shows that there is a ramp that allows further reduction of the volume within which the cell 950 resides. The values of z1, z2, z3 are chosen to guide the cluster to a desirable location in the well.

A shape that can be formed by this two dimensional constriction can be a conical shape. Alternatively, a pyramidal shape is able to be formed for the volume that the cell occupies. The ramp structure in the z-dimension can occur in various combinations: a ramp at the bottom of the well, a ramp at the top of the well-guides the cell cluster to the sensing unit aperture, a ramp both at the top and bottom of the well. Given that the top of the well is a function of how much media has been placed in the well, there are advantages to guiding the cell cluster to the bottom of the well. Guiding the cluster to the bottom also improves the quality of images one can obtain. The sensing unit position is able to be adjusted accordingly (for example when doing impedance measurements).

FIG. 9J illustrates top views of a collapsible well according to some embodiments.

Fluidic channels 930 can be added to the constricting walls 926′ to allow for fluid exchange between one side of the chamber and its other side. An electrode 932 is able to be added to the semi-rigid wall 924. A fluidic channel 930 of a certain diameter allows fluid exchange between one side of the chamber and its other side. If the fluidic channel 930 is sufficiently large, it will allow expelling cells that are smaller than a certain size from the measurement side of the well to the inactive side of the well. An electrode 932 allows an impedance measurement to take place between the electrode 932 and the sensing unit 900 which can have a corresponding electrode for the other terminal that is needed for this type of measurement. The electrodes are non-occluding for a camera, so they do not have to be transparent. The electrode 932 on one side (semi-rigid wall 924) and the electrode on the other side (sensing unit 900) can be composed of many individually addressable electrodes. This allows a multi-directional impedance measurement process on the cell cluster. Chemical sensors can be arranged horizontally and vertically to provide measurements of diffusion and gradients.

The moveable wall 924 has been shown as a flexible membrane/wall/curtain. This allows it to adapt to the shape of the constricting zones 926′ and allows better capability to guide the cell cluster 950 to an optimal zone. If there was a rigid structure, it would limit the flexibility of adjusting the final micro-well size and shape. One can still have a fixed shape for the brown membrane/wall/curtain. This limits the flexibility of the final shape and size of the micro-well. A combination of flexible and rigid membrane/wall/curtain is shown.

FIG. 9K illustrates top views of a collapsible well according to some embodiments. A hybrid of a rigid structure and a flexible structure for the moveable wall 934 is shown. The hybrid moveable wall 934 is able to fit within the constricting walls 926′, where the flexible aspect is able to bend with the constricting walls 926′ while the rigid structure maintains its form.

The following applies for variants of the collapsible well shown above. The mechanism to change the well size is implemented through a piezo element 920 that also has a driver and a mechanism to couple the piezo element's change in size to a shaft structure. The coupling between the piezo element and the shaft structure can go through a mechanical amplification as well. Alternative implementations using classical electromechanical methods are also applicable. The camera system under the well can act as a feedback system that provides instructions to the piezo driver or the electro-mechanical element as to how far to advance the well wall. A user interface can be generated that can do any combination of the following: adjust the progression rate of the wall so as to not disturb the cell cluster, and adjust the final position of the wall so that it conforms to user specified parameters (e.g., distance of one edge of the cluster from the sensor, distance between the cluster and the wall, well size, and others). This allows for optimization and flexibility in adjusting the well size. In some embodiments, the chemical sensors are on the side-wall, an imaging system is on the bottom of the well, a collapsible well wall emerges from the side wall, and impedance measurement electronics are on the face of the collapsible well wall.

FIG. 9L illustrates top views of a collapsible well according to some embodiments. The moveable/collapsible wall 934 can be implemented on the lid 936 of the well array-the electro-mechanical unit and shaft system 920 comes from the top of the well and can be part of the well lid 936. The chemical sensors 900 can be on the well wall.

FIG. 9M illustrates top views of a collapsible well according to some embodiments. The chemical sensors 928 can be attached to the moveable wall 934 that comes from the top, and an imaging unit 940 is able to be included in a wall.

The constricting walls 926′ that allow the formation of the micro-well will then have a large opening near the top of the well and a small opening near the bottom of the well. This allows the guiding of the cell cluster 950 to a smaller volume near the bottom. The microchannels 930 that allow fluid to escape from the micro-well can be part of the constricting structure 926′. The collapsible wall 934 that comes from the top can be made of transparent material to allow lighting for the imaging system 940 to come from the top. The electrode structure for measuring impedance can be on the bottom of the well and a complementary electrode structure can be attached to the shaft that moves the collapsible wall 934.

This approach may consider using multiple data and/or information that are captured from Genius Wells™ and/or an instrument to improve optical imaging or image processing, including, but not limited to, electrical (such as impedance), biochemical, or optical modalities in order to improve sensitivity of motion detection or tracking motion. A method of implementing a collapsible well is able to comprise positioning a sample in the collapsible well. Selectively engaging a piezo element 930 causing a collapsible wall 934 to collapse a size of the well. In some embodiments, engaging the piezo element 930 comprises sending an electrical signal to a piezo motor that causes a piezo shaft to push the collapsible wall 934 away from the well wall. One or more characteristics of the sample within the collapsed well are able to be detected using one or more sensors in and/or around the well. The piezo element 930 is selectively engaged to cause the collapsible wall 934 to increase/restore a size of the well. In some embodiments, engaging the piezo element 930 comprises sending an electrical signal to a piezo motor that causes a piezo shaft to pull the collapsible wall 934 toward the well wall.

### Depth Calibration Mechanism for Optical Imaging

FIG. 9N illustrates a side view of a mechanism for more accurate depth calibration for the imaging system according to some embodiments. A mechanism 960 for more accurate depth calibration for the imaging system is shown. The mechanism 960 includes calibration spheres 962. Knowing the z-position of any given cell is useful for poly-sensing applications. Currently, the x and y positions are relatively easy to determine based on the optical imaging system that looks through the bottom of a transparent well.

Components 964 and 966 are added for multiple purposes: to give the structure a pre-determined incline angle so a lateral position can be matched to a vertical position, and to provide mechanical stability for insertion into the well.

Component 964 can be lined up to one of the well walls. Component 964 can have a wide rectangular cross-section because it does not generate any optical occlusion and can prevent the insert from tilting over-so it provides mechanical stability in the well. Component 966 has a minimal depth (enough to provide mechanical stability and also to minimize optical occlusion even though it can be made of optically transparent material). Component 968 is of sufficient width to give the spheres 962 mechanical stability, but not too large as to generate an optical occlusion for cells that may be floating above it. The components 964, 966 and 968 can all be optically transparent but should be non-conductive in order to not disturb the impedance measurements.

FIG. 9O illustrates a top view of a mechanism for more accurate depth calibration for the imaging system according to some embodiments. The mechanism 960 for more accurate depth calibration for the imaging system is shown.

One can generate an insert for the well which has the following characteristics. It has features that are of similar size to a cell. For example, a feature is a sphere that has a 20 um diameter. Other sphere sizes can be used as well. These spheres will have similar optical properties to the cells being imaged. The intention is to have a proxy for the cells that are being imaged (similar optical characteristics from an imaging perspective, and similar in size range).

The calibration spheres 962 can be placed inside a well (maybe adjacent to a wall of the well to minimize occlusion of the actual cells that are being imaged).

The calibration spheres 962 are arranged on an insert where their z position is known. The spheres 962 are arranged on an incline where by determining the y position of the sphere, and the z position is known. This allows the imaging system to know the exact z position of a calibration sphere. It further allows the imaging system to acquire pictures of this known z-position sphere using different focal depths. For example, as the focal depth of the imaging system is being adjusted, the sphere of known z position will produce different images. This information can be used as a reference for determining the position of cells in the media.

Example constructions of such an insert are presented. The variables that are available in such a construct are: a dimension of the incline insert (can be modified for different well sizes), the number of calibration spheres per insert, the diameters of calibration spheres per insert, the permutations of different sphere sizes on a given insert, a further permutation is that the depth calibration marks can be added to the insert where they come into focus at a known value of z.

There are many variations for the insert. The structure can be a stand-alone insert or it can be part of the lid structure for the well (attached to the bottom surface of the lid). If attached to the lid, the insert can be attached through a simple spring that forces the calibration structure to the bottom surface of the well while allowing the lid to be closed adequately. The insert is shown as a linear ramp that goes from one side of the well to the other side. The insert can still be a linear ramp but with a smaller lateral dimension-so it does not have to extend from one side of the well to the other. This will allow for easier insertion into the well, it will reduce the optical occlusion, and it will make it applicable to a larger range of well sizes. The insert can also be constructed in the form of a spiral whose top view will form a circle or semi-circle. The advantage of this type of structure is that it can be more easily tucked into a corner of the well. It may also provide a more robust mechanical construction. In general, any shape that has a unique association between the z dimension and a unique x, y coordinate will serve the intended purpose. One constraint is that the spheres cannot be located above one another for a given (x, y) coordinate. Therefore, the shape is not constrained to ramps or spirals. Other contours that satisfy the above conditions will also work.

More generally, the calibration objects can be spheres of varying (but known) sizes. They can even be other solid shapes (cubes, inverted pyramids, others). The calibration objects should be similar in optical properties to the cells. Additionally, they could have markings that further help the calibration process. This mechanism allows for interpolation between the spheres to achieve better estimation of depth between the different levels of the spheres. In the transition from focus from the bottom of the sphere to the equator of the sphere, further information can be gathered about depth from the amount of departure from sharp focus.

### High Throughput Polybiosensing-Imaging System

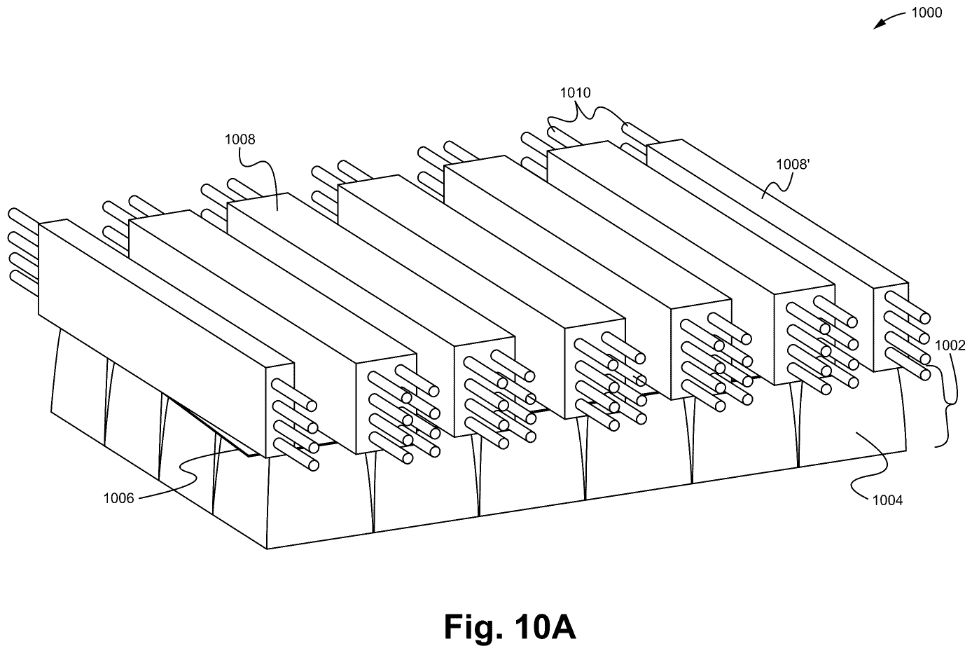

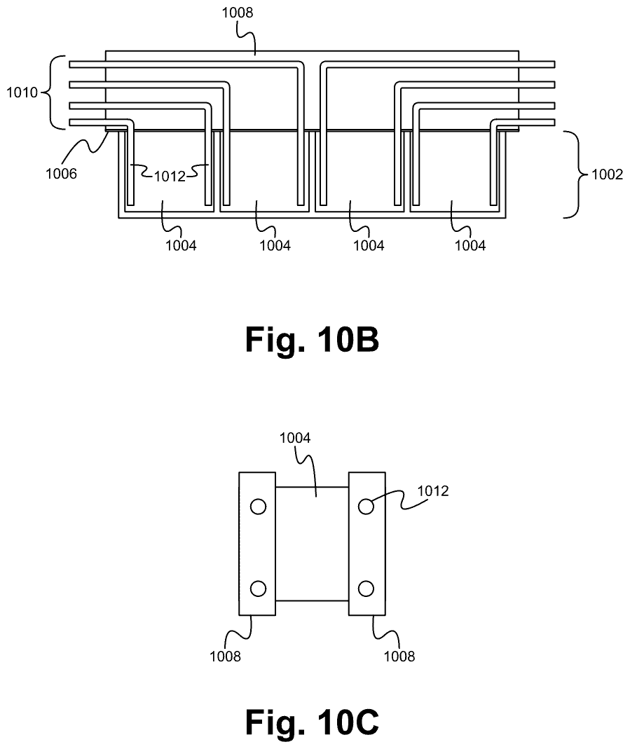

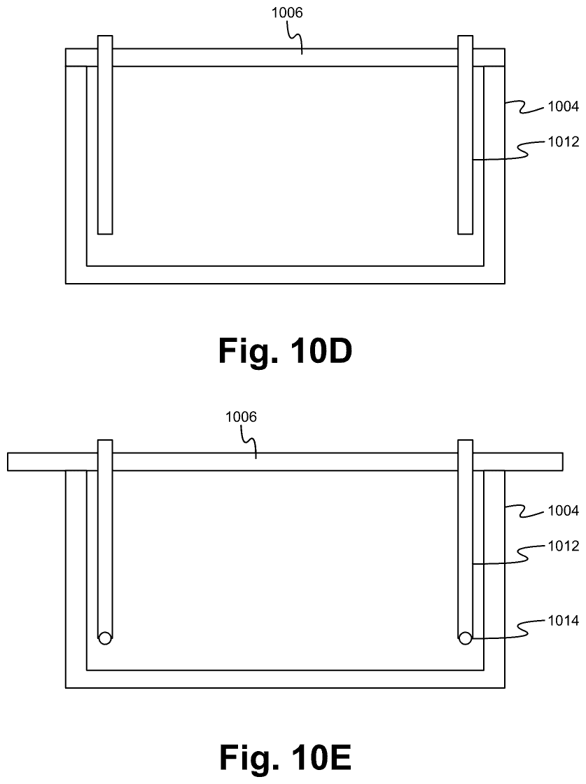

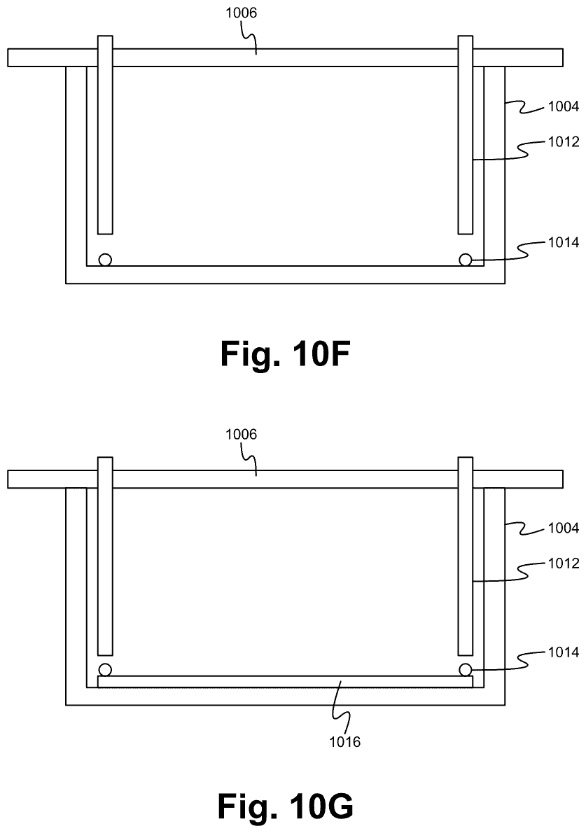

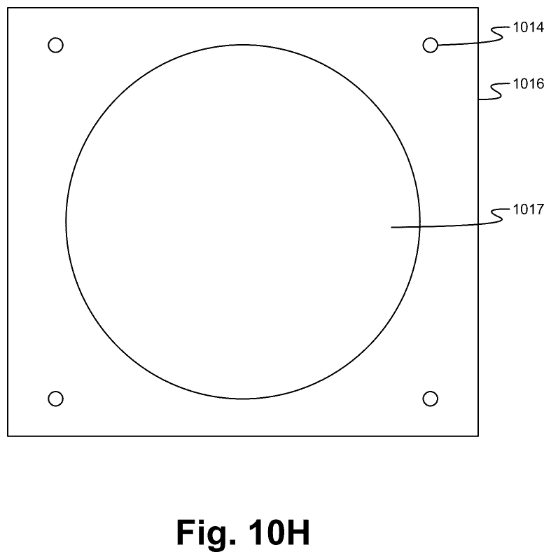

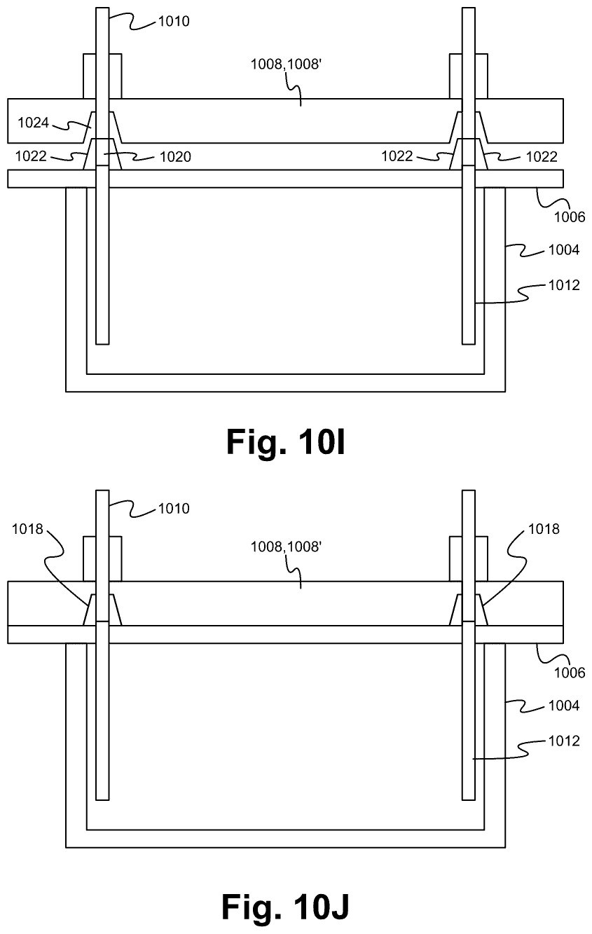

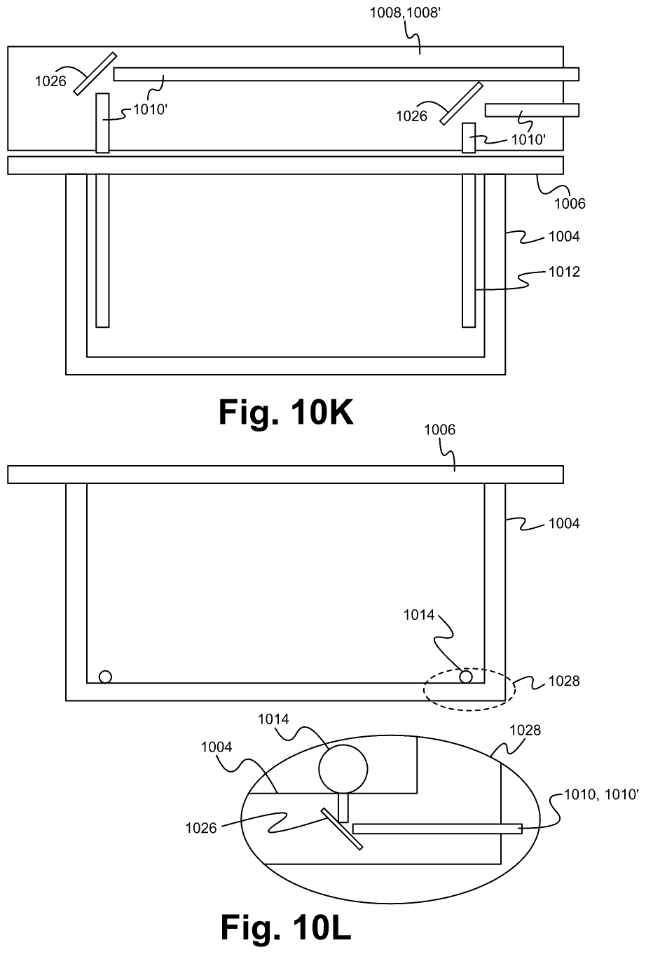

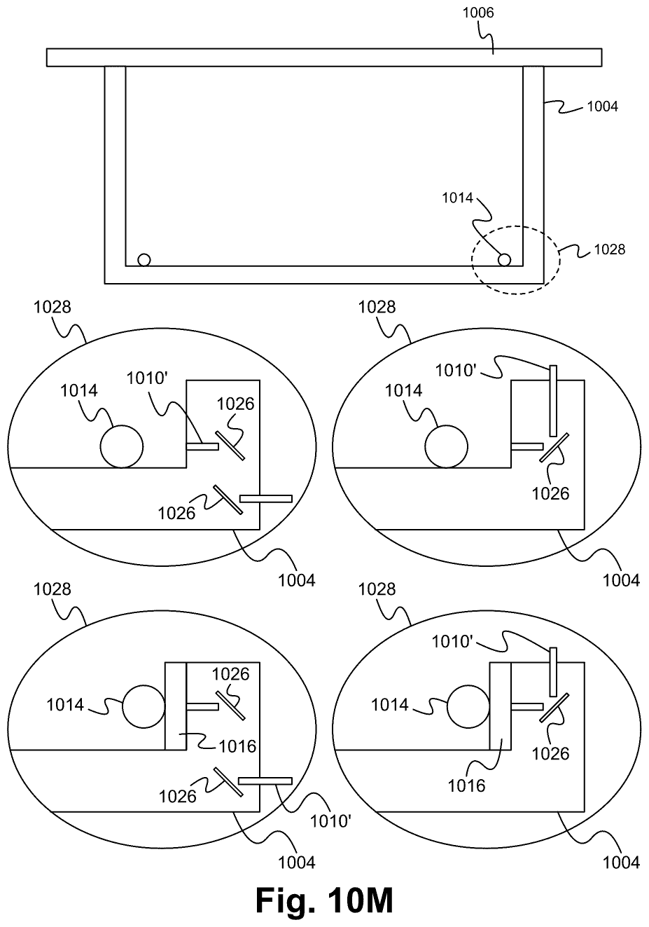

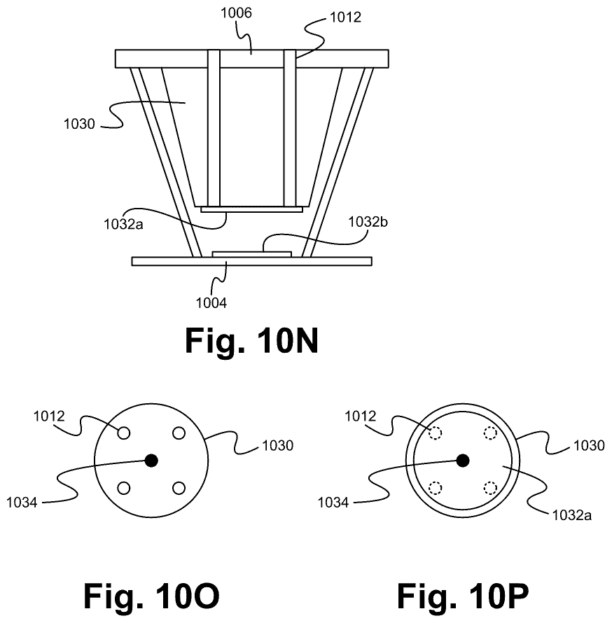

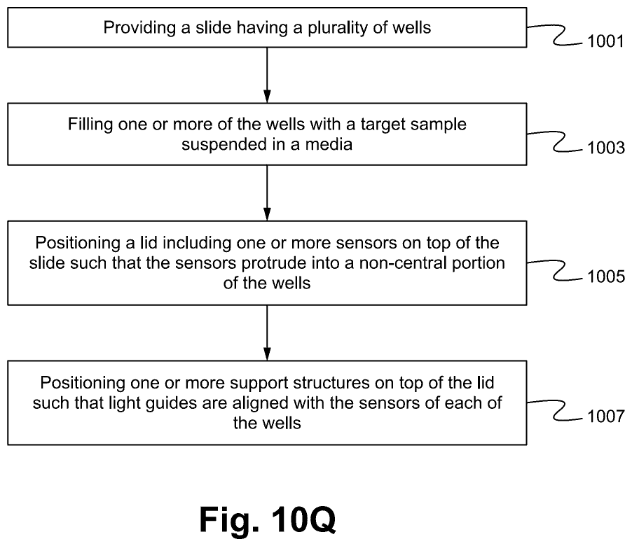

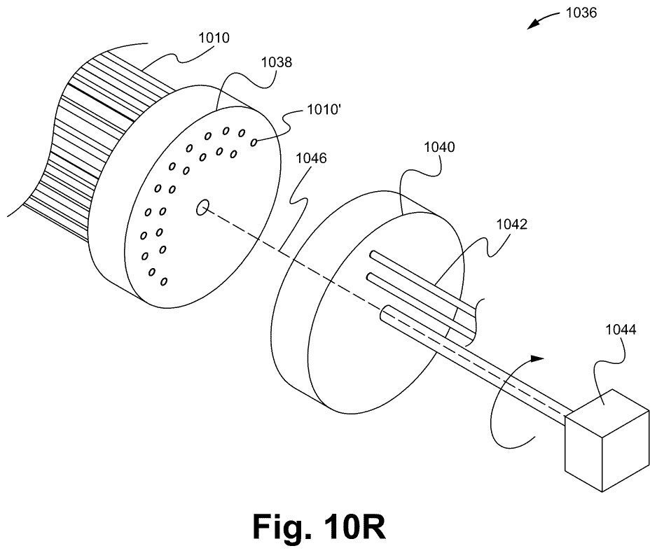

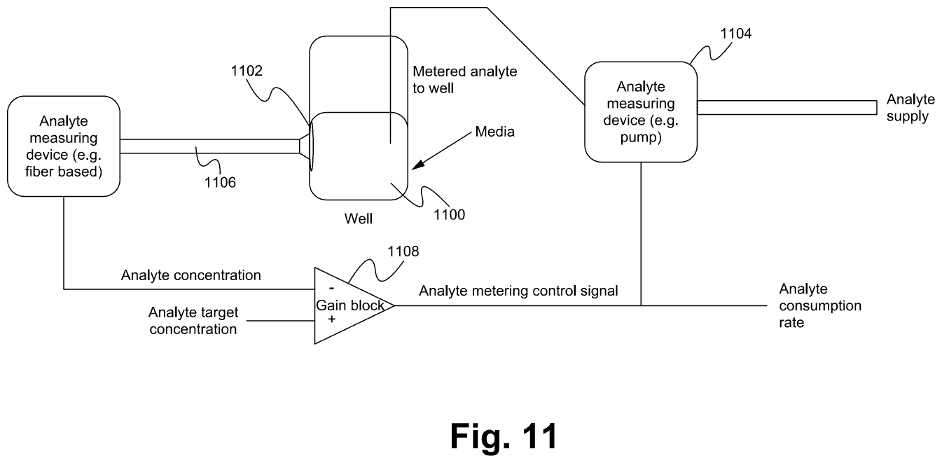

FIGS. 10A, 10B and 10C illustrate perspective, side cross-sectional and top cross-sectional views, respectively, of a polybiosensing-imaging system 1000 for high throughput according to some embodiments. The polybiosensing-imaging system 1000 of FIGS. 10A-10R is able to be substantially similar to the system 10 except for the differences described herein. For example, the features of the wells, slides, light guides, sensors, inserts and/or imaging units described with respect to FIGS. 10A-R are able to be incorporated into the wells, slides, light guides, sensors, inserts and/or imaging units of the other figures described herein. As shown in FIGS. 10A-10R, the system 1000 comprises a well slide 1002 including a plurality of wells 1004, a lid 1006 including a plurality of sensors 1012 and a plurality of sensor support structures 1008 including one or more light guides 1010. The lid 1006 is able to be detachably coupled to the top of the wells/well slid 1002 (e.g. via one or more clamps (not shown)). The support structures 1008 are able to be coupled to a top of the lid 1006 (e.g. no air gap). Alternatively, the support structures 1008 and the lid 1006 are able to form a single integrated lid. In some embodiments, the bottom of the support structures 1008, 1008′ is able to include a flexible circuit for electrical connections (e.g. impedance measurements), heating elements, light emitting diodes (e.g. as an illuminator), temperature sensors and/or other types of sensors. In such embodiments, these sensors are able to be positioned above the wells 1004 along with the structures 1008, 1008′ in order to access and/or detect characteristics of the media within the wells 1004.

The well slide 1002 provides side wall and bottom wall structure for a plurality of wells 1004. The well slide 1002 is able to be made of an optically transparent material, such as glass or plastic. In some embodiments, as shown in FIG. 10A, the well slide 1002 has 6×4 array of individual wells 1004. Alternatively, the well slide 1002 is able to have any size array of wells 1004. The wells 1004 are able to be arranged in a variety of patterns and/or have a variety of widths, lengths and heights. For example, in some embodiments each well 1004 has a width and length of 16.3 mm. Although the configuration shown in FIG. 10 shows wells 1004 having the same size and spacing in a grid pattern, it is understood that slides 1002 are able to be configured with wells 1004 having different sizes and patterns. Additionally, although the wells 1004 are illustrated as having a square bottom profile, they are able to have other shaped bottom profiles including circular, oval, triangular or any other shape. Although not shown in FIGS. 10A-C for the sake of clarity, in some embodiments the bottom of the slide and/or wells is coupled with/adjacent to an electrical interconnect substrate 12. Alternatively, an electrical interconnect substrate 12 is able to be omitted or coupled with/adjacent to the side or top of slide and/or one or more of the wells. Alternatively, electrical interconnects are able to be integrated within the wells 1004 or otherwise operatively coupled with the wells 1004 as described herein.

As shown in FIGS. 10A-C, the system 1000 includes two edge sensor support structures 1008′ and N-1 interior sensor support structures 1008 (where N is the number of columns of wells 1004 within the slide 1002). Thus, as the number of columns of wells 1004 increases, the number of interior sensor support structures 1008 increases, but the number of edge sensor support structures 1008′ remains the same. Further, both the middle sensor support structures 1008 and the edge sensor support structures 1008′ include at least one light guide 1010 for each well 1004 they are positioned over. Thus, due to the four wells 1004 in each row of the slide 1002, the interior sensory support structures 1008 (which straddle two rows) include at least eight light guides 1010, and the edge sensor support structure 1008′ include at least four light guides 1010. As also shown in FIGS. 10A-C, for both the interior sensor support structures 1008 and the edge sensor support structures 1008′, the light guides 1010 for each row of wells 1002 are vertically stacked within structures 1008, 1008′. As a result, as the number of wells 1002 in each row (and/or the number of light guides 1010/sensors 1012 needed for each well 1002) increases, a height of the support structures 1008, 1008′ is able to increase in order to accommodate the greater number (and higher stack) of light guides 1010.

The light guides 1010 are able to co-molded with the support structures 1008, 1008′ and/or have less or equal to a maximum curvature in order to minimize signal loss of optical signals transmitted through the light guides 1010. As shown in FIGS. 10B and 10C, the light guides 1010 are accessible from a perimeter of the structures 1008, 1008′ (for transmitting and/or receiving signals to/from the external electronic devices 8), travel through the structures 1008, 1008′ and then extend downward to a bottom surface of the structures 1008, 1008′ such that they are each aligned with a different one of the sensors 1012 of the lid 1006. The portion of the light guides 1010 that is accessible from the perimeter of the support structures 1008, 1008′ is able to be configured to couple with an optical measurement unit of the devices 8 via one or more fiber cables.

The position of the sensors 1012 within the lid 1006 and the position of the light guides 1010 within the structures 1008, 1008′ is able to be configured such that the light guides 1010/sensors 1012 are aligned with/extend into a perimeter or non-central portion of the wells 1004 (e.g. the corners, edges). In particular, this non-central positioning prevents the light guides 1010 and sensors 1012 from obscuring view of target sample within the central portions of the wells 1002. Although as shown in FIG. 10C, the sensors 1012 are evenly or symmetrically spaced along the perimeter of the well 1004 in each of the corners, it is understood that the sensors 1012 are able to be unevenly, asymmetrically or otherwise positioned along the perimeter of the well 1004 (in or out of one or more of the corners).

As shown in FIG. 10C, each of the sensors 1012 (in combination with sensor dots described below) is able to be associated with a type of sensing including one or more of pH, oxygen (O2), glucose, carbon dioxide (CO2), secretome, lactate (or other metabolites), mechanical sensing (e.g. via acoustic signals), temperature and/or any other chemistry that is able to be detected via optical fibers or amperometric/solid state sensors. For example, as shown in FIG. 10C, the four sensors 1012 (in combination with sensor dots described below) are configured to sense pH, O2, glucose and one of the other types (XX).

FIG. 10D illustrates a close up side cross-sectional view of the lid 1006 and one of the wells 1004 according to some embodiments. As shown in FIG. 10D, the sensors 1012 are able to extend through apertures of the lid 1006 into the perimeter of the well 1004. In some embodiments, the sensors 1012 at least partially protrude above the lid 1006. Alternatively, one or more of the sensors 1012 are able to be flush with a top of the lid 1006. In some embodiments where sensor dots are positioned near the bottom of the well 1004, each of the sensors 1012 are able to have a length such that a bottom of the sensor 1012 is adjacent to the bottom of the well 1004 where an associated sensor dot is located. Alternatively, where the sensor dot is coupled to a bottom of the sensor 1012, the sensors 1012 are able to be shorter in length (e.g. extend to a middle depth of the well 1004).

The sensors 1012 are each able to be a gradient index rod lens (GRIN rod). Alternatively, one or more of the sensors 1012 are able to be an optical fiber and/or a combination of a plurality of optical fibers. Further, in some embodiments the optical fiber and/or combination of a plurality of optical fibers is able to be protected by an outer jacket that surrounds a perimeter of the fiber(s) such that only the ends of the fiber(s) are exposed. In some embodiments, the same sensor 1012 is able to server as both the excitation source (e.g. transmit light to a corresponding sensor dot) and the receiver of the fluorescence signal from the sensor dot (e.g. receive light from the corresponding sensor dot). Alternatively, two or more sensors 1012 are able to be operably grouped together, wherein a first number of sensors 1012 of the sensor group operate as the excitation source for a corresponding sensor dot, and remaining number of the sensors 1012 of the sensor group operate as the signal receiver for the corresponding sensor dot. In such embodiments, the group of sensors are able to be positioned adjacent to each other (e.g. as close as possible) and/or angled in a manner such that all are pointing toward the same chemical sensor dot. Thus, although not illustrated for the sake of brevity, in such embodiments the figures described herein showing a single sensor 1012 would be replaced with a sensor group as described. In some embodiments, one or more of the sensors 1012 are able to include a lens coupled to a bottom tip (e.g. aligned with the optical fiber(s)) focused on the corresponding sensor dot (when the sensor 1012 is fully positioned within the well 1004). Indeed, in such embodiments this feature enables a more efficient collection of signals from the chemical sensor dots that are at a fixed distance from the end of the sensors 1012.

In some embodiments, the top of the light guides 1010 where they exit the structures 1008, 1008′ away from the well 1004/lid 1006, is able to have a shape different than a bottom of the sensors 1012. For example, a cross-section of the top of the light guides 1010 (about their central axis) is able to be circular, whereas a cross-section of the bottom of the sensors 1012 (about their central axis) is able to be square or other two-dimensional shape (e.g. to match the shape of the chemical sensor dots 1014). This transition in cross-sectional shape is able to take place along the length of the light guides 1010, the sensors 1012 or both. For example, the bottom of the light guides 1010 is able to match the cross-sectional shape of the bottom of the sensors 1012 such that the sensors 1012 do not need to change their cross-sectional shape from top to bottom. Alternatively, the bottom of the light guides 1010 is able to wholly or partially not match the cross-sectional shape of the bottom of the sensors 1012 such that the top of the sensors 1012 needs to match the cross-sectional shape of the bottom of the light guides 1012 and then at least partially change in cross-sectional shape from top to bottom to match the shape of the sensor dots 1014.

FIG. 10E illustrates a close up side cross-sectional view of the lid 1006 and one of the wells 1004 with sensor-attached chemical sensor dots 1014 according to some embodiments. As shown in FIG. 10E, the sensors 1012 are each able to have a chemical sensor dot 1014 coupled to a bottom of the sensor 1012. As a result, the sensors 1012 are able to automatically be adjacent to the chemical sensor dot 1014 for providing excitation and signal reception from the dot 1014. Thus, in such embodiments the sensors 1012 do not need to be positioned adjacent to the bottom of the well 1004. If separate sensors 1012 are used for excitement and signal reception (as described above), the dot 1014 only needs to couple to the bottom of one of the sensors 1012.

FIG. 10F illustrates a close up side cross-sectional view of the lid 1006 and one of the wells 1004 with well-attached chemical sensor dots 1014 according to some embodiments. As shown in FIG. 10F, the well 1004 is able to have one or more chemical sensor dots 1014 printed, placed or otherwise coupled to a bottom wall of the well 1004 (e.g. non-centrally positioned). FIG. 10G illustrates a close up side cross-sectional view of the lid 1006 and one of the wells 1004 including an insert 1016 having indicators or chemical sensor dots 1014 according to some embodiments. As shown in FIG. 10G, the insert 1016 is able to be positioned on the bottom of the well 1004 and is able to have one or more chemical sensor dots 1014 printed, placed or otherwise coupled to a top of the insert 1016 (e.g. non-centrally positioned). The insert 1016 is able to be optically transparent, have dimensions that match a profile of the bottom of the well 1004 (so that it cannot change position once within the well 1004) and/or be made of plastic. As shown in FIG. 10H, in some embodiments the insert 1016 is able to have one or more holes 1019 in a middle portion to avoid occluding optical imaging of the target sample within the well 1004 from the bottom of the well 1004.

The insert 1016 fits within the well 1004 so as to position sensing components in physical contact with the well 1004 contents, such as a fluid and/or culture media. The inserts 1016 can have various sizes depending on the well size, and can include various types of sensors described herein. The inserts 1014 can be made from a transparent material. The insert 1014 form factors and sizes are modular. The insert 1014 can be designed according to many different dimensions, and multiple sensor modalities can be implemented. The surface of inserts 1014 can be functionalized with biocompatible chemical moieties to enhance long-term stability and biocompatibility. It is understood that the inserts 1014 can be configured to use different sensor types or to include additional different sensor types. The variety of different sensor types can be coupled to the inserts 1014, and if desired, to the well 1004 side walls and bottom wall for 2D and 3D continuous sensing of multiple different modalities including all of the different modalities described herein.

FIGS. 10I and 10J illustrate separated and coupled close up side cross-sectional views, respectively, of the lid 1006, the support structure 1008, 1008′, one of the wells 1004 and alignment protrusions 1018 according to some embodiments. As shown in FIGS. 10I and 10J, one or more alignment protrusions 1018 are able to be positioned on top of each of the sensors 1012. In some embodiments, the protrusions 1018 are coupled to and/or integrated into the top of the lid 1006. Alternatively, the protrusions 1018 are able to be coupled to the top of the sensors 1012 (and positioned flush with the top of the lid 1006 when the sensors 1012 are positioned fully within the apertures of the lid 1006). The alignment protrusions 1018 are able to have a central channel 1020 that surrounds and provides aligned access to the associated sensor 1012 and tapered sides 1022. In some embodiments, the protrusions 1018 are conical as the tapered sides 1022 extend radially around the channel 1020/sensor 1012. Alternatively, the tapered sides 1022 are able to have one or more edges such that the protrusions 1018 have a pyramid or other shape.

The bottom of the support structures 1008, 1008′ are able to have recesses 1024 that surround the bottom of each of the light guides 1010. Further, the recesses 1024 are able to have a complementary shape to that of the protrusions 1018 such that the protrusions 1018 precisely fit within the recesses 1024 when the bottom of the light guides 1010 fully slide into the channels 1020 and abut the top of the sensors 1012 as shown in FIG. 10J. Indeed, the tapered sides 1022 and complimentary recesses 1024 provide the advantage of facilitating easy alignment and coupling of the support structures 1008, 1008′ onto the lid 1006 such that each of the light guides 1010 is aligned with a corresponding one of the sensors 1012.

FIG. 10K illustrates a close up side cross-sectional view of the lid 1006, the support structure 1008, 1008′ having discontinuous light guides 1010′, mirrors 1026 and one of the wells 1004 according to some embodiments. As shown in FIG. 10K, a mirror (or other light reflective structure) 1026 is able to be positioned within the support structures 1008, 1008′ to reflect and thereby transmit light between two sections (e.g. vertical and horizontal sections) of the light guides 1010′. Alternatively, the mirrors 1026 are able to be replace or supplemented with one or more lenses that transmit or refract the light between the two sections of the light guides 1010′. In such embodiments, the support structures 1008, 1008′ are able to be transparent to light in between the two sections of the light guides 1010′ and the mirrors 1026. Alternatively, the support structures 1008, 1008′ are able to have a cavity between the two sections of the light guides 1010′ and the mirrors 1026 to enable the passage of light. In some embodiments, the vertical section of the light guides 1010′ are able to be omitted and the support structures 1008, 1008′ are able to be transparent to light or have cavity in between the horizontal sections of the light guides 1010′, the mirrors 1026 and the top of the sensors 1012.

FIG. 10L illustrates a close up side cross-sectional view of the wells 1004 including different well light guide structures according to some embodiments. As shown in FIG. 10L, the bottom and/or side walls of one or more wells 1004 of the slides 1002 are able to have one or more well light guides structures 1028. In particular, the well light guides structures 1028 are able to be substantially similar to the light guides 1010, 1010′ and the support structures 1008, 1008′ except that the support structures 1008, 1008′ are replaced with the walls of the wells 1004 and the light guides 1010, 1010′ (and associated mirrors if used) are aligned with the sensor dots 1014 (as printed on the inside of the well or on an insert positioned in the well) from the bottom or side of the well 1004 instead of with the sensors 1012 from the top. If approaching from a side of the well 1004, the light guides 1010, 1010′ are able to curve to a vertical direction under the sensor dots 1014 or utilize mirrors 1026 to receive signals from the sensor dots 1014. In such embodiments, the sensors 1012 are able to be omitted or incorporated into the end of the light guides 1010, 1010′ that is proximate the sensor dot 1014. Like with the support structures 1008, 1008′, the ends of the light guides 1010, 1010′ that extend away from the sensor dots 1014 are able to be accessible from a side or bottom of the well 1004 (e.g. in stacked formations) for operably coupling with the optical measurement unit. Alternatively, they are able to be routed to the top of the well 1004 (through a side wall) and/or couple with the lid 1006/support structure 1008, 1008′ like the sensors 1012 described above. Also like with the sensors 1012 and dots 1014, the light guides 1010, 1010′ are able to be positioned along a perimeter (e.g. non-central) portion of the wells 1004 so as to not occlude view of the sample within the wells 1004.

FIG. 10M illustrates alternate embodiments of the well light guide structures 1028 according to some embodiments. As shown in FIG. 10M, the structures 1028 are able to utilize multiple mirrors (and/or lenses) 1026 (see top left and bottom left embodiments), sensor dots 1714 printed on a side wall or printed on an insert 1016 on the side wall (see bottom left and bottom right embodiments), light guides 1010, 1010′ routed vertically up one of the side walls (see top right and bottom right embodiments), or a combination thereof. Like in FIG. 10L, the well light guides structures 1028 are able to be substantially similar to the light guides 1010, 1010′ and the support structures 1008, 1008′ except that the support structures 1008, 1008′ are replaced with the walls of the wells 1004 and the light guides 1010, 1010′ (and associated mirrors if used) are aligned with the sensor dots 1014 (as printed on the inside of the well or on an insert positioned in the well) from the bottom or side of the well 1004 instead of with the sensors 1012 from the top. Additionally, in each of the structures 1028, the mirrors 1026 are able to be replaced or supplemented with lenses and/or curves of a continuous light guide 1010, 1010′ and/or the support structures 1008, 1008′ are able to be transparent to light in between the two sections of the light guides 1010′, the mirrors 1026 and/or the dots 1014. Again, the ends of the light guides 1010, 1010′ that extend away from the sensor dots 1014 are able to be accessible from a side or bottom of the well 1004 (e.g. in stacked formations) for operably coupling with the optical measurement unit. Alternatively, they are able to be routed to the top of the well 1004 (through a side wall as shown) and/or couple with the lid 1006/support structure 1008, 1008′ like the sensors 1012 described above.

FIG. 10N illustrates a side cross-sectional view of the lid 1006 and one of the wells 1004 with a lid protrusion 1030 according to some embodiments. FIGS. 10O and 10P illustrate a top and bottom view, respectively, of a lid protrusion 1030 according to some embodiments. As shown in FIGS. 10N-P, the lid 1006 is able to have one or more lid protrusions 1030 that extend downward from the lid 1006 into the well 1004. The lid protrusions 1030 are able to be similar in shape to the alignment protrusions 1018 except without a central channel. For example, the protrusions 1030 are able to have conical shape or pyramid shape created by angled sides. Alternatively, the lid protrusions 1030 are able to have parallel sides such that they have a cylindrical, square, rectangular or other parallel sided shape. In some embodiments, the side walls of the wells 1004 are also able to be angled (instead of parallel) to each other. As a result, like the lid protrusions 1030, the wells 1004 are able to form a conical, pyramid or other angle-sided shape. In such embodiments, where both the lid protrusions 1030 and the well walls are similarly angled, they are able to facilitate insertion and/or alignment of the lid protrusions 1030 within the wells 1004.

The sensors 1012 are able to be positioned through the lid protrusions 1030 and the lid 1006 above corresponding sensor dots 1014 (not shown) within the well 1004. Alternatively, the sensors 1012 are able to be omitted and a well light guide structure 1028 is able to be incorporated into the well 1004 in order to detect signals from and deliver excitation to the chemical sensor dots 1014 in the well 1004. As also shown in FIGS. 10N-P, a first electrical terminal 1032a is able to be coupled to the bottom of the lid protrusion 1030 and a second electrical terminal 1032b is able to be coupled to a bottom of the well 1004. Alternatively, the first electrical terminal 1032a is able to be embedded within the lid protrusion 1030. Alternatively, both the first and second terminals 1032a, 1032b are able to be in the bottom of the well 1004. The benefit of having the first terminal 1032a on the outside of the protrusion 1030 is that it is in direct contact with the well fluid. The down side is that it can rub against the well side walls when inserted. The benefit of having the first terminal 1032a inside of the protrusion 1030 is that it will be better protected mechanically, but the electrical fields will have to go through the protrusion holes 1034. In any case, the first and second electrical terminals 1032a, 1032b are configured to measure an electrical impedance of the sample between the two terminals. In particular, the terminals 1032a, 1032b are able to be electrically coupled with the devices 8 (e.g. via the flexible circuitry described above) for providing the impedance values for the well 1004. Further, in some embodiments the terminals 1032a, 1032b are able to be gold and/or optically transparent so as to not occlude the middle of the well 1004.

In some embodiments, a bottom of the lid protrusion 1030 includes a hole 1034 that allows fluid displacement and provides a conduction path for the impedance measurement. Alternatively or in addition, the lid protrusion 1030 is able to include a plurality of holes 1034 on the bottom and/or sides of the protrusion 1030. In some embodiments, the holes 1032 are positioned on a side of the protrusion 1030 proximate the lid 1006. In some embodiments, the holes 1034 have the same size. Alternatively, one or more of the holes 1034 are able to be different sizes. In some embodiments, a size of the holes 1034 increases the closer the hole 1034 is to the lid 1006 to control fluid exchange and/or diffusion paths. In some embodiments, the lid protrusion 1030 is able to include one or more LEDs that protrude from a bottom of the protrusion 1030 (e.g. instead of or adjacent to the hole 1034) for providing light to the wells 24. The lid protrusion 1030 displaces fluid in the well 1004 to reduce the volume of the culture media/analyte/fluid thereby increasing the ratio of cell to liquid volume. The excess fluid is able to flow around the lid protrusion 1030 and into a collection well. Specifically, the excess fluid is able to flow into the lid protrusion 1030 through the holes 1034. As a result, by modulating the number and size of the holes 1034 and the size of the lid protrusion 1030, the system is able to control the sample to fluid ratio. Accordingly, the lid protrusions 1030 provide the advantage of enabling control of the ratio of sample (e.g. cells) to fluid/culture media as well as enabling conductive electrodes to be positioned in close proximity to each other. Indeed, for assays with a required latency to achieve a certain density of cells, smaller wells produced by the protrusions help to achieve that density sooner.

As a result, the system 1000 provides the advantage of parallelizing the system with the sensor support structures 1008 enabling more simultaneous analyses and efficient upscaling. Further by providing each well with a plurality of different types of sensors, the system enables one assay with “n” separate sensors leading to fewer and shorter number of assays that currently must be performed.

In some embodiments, the system 1000 includes an imaging stage (e.g. imaging unit 36 and/or devices 8) that is configured to optically interrogate (e.g. excite via transmitted optical signals and/or read via receiving optical signals from) the chemical sensors (sensor dots 1014) of each of the wells 1004. In some embodiments, the imaging stage is able to be optically coupled with each of the light guides 1010 in parallel and thereby transmit light to and receive the light fluoresced from each of the sensor dots 1014 for measuring the chemistry within the wells 1004 that the sensor dots 1014 are associated with. For example, a bundle of optical fibers each in optical communication with one or more light sources and/or readers are able to be arranged in an array format that matches the well geometry on the slide 22 and thus is aligned to direct light to and receive light from each sensor 1014 within/on each well 24 of the slide 22. As described herein the optical fibers are able to access the sensors/wells via the top (e.g. through the lid), bottom and/or sides of the wells 24. In some embodiments, the optical fibers are able to be replaced with one or more optical lenses for directing light to and receiving light from the sensors 1014 (e.g. with bright field or fluorescent light). For example, the lenses are able to be a part of the imaging unit 36. Alternatively, the optical fibers are able to be omitted and the light sources and/or light readers are able to directly access (e.g. be in optical communication with) the sensors 1014.

Alternatively, the imaging stage is able to have a movable interrogator portion such that the interrogator optically interrogates only a subset of the light guides 1010/sensor dots 1014 at a time and sequentially moves through each of the subsets until all of the light guides 1010/sensor dots 1014 have been interrogated. For example, as shown in FIG. 10R, the interrogator 1036 is able to comprise a fixed light guide wall 1038 that receives a distal end 1010′ of the light guides 1010, a rotatable reader plate 1040 including a plurality of interrogator optical fibers 1042, and a rotation mechanism 1044 that is able to rotate the reader plate 1040 about an axis 1046. In particular, the distal ends 1010′ of light guides 1010 that are optically operable with a first same type of sensor 1014 (e.g. pH) are all able to be positioned around the point where the axis 1046 intersects the wall 1038 at a first radial distance r, the distal ends 1010′ of light guides 1010 that are optically operable with a second same type of sensor 1014 (e.g. O2) are all able to be positioned around the point where the axis 1046 intersects the wall 1038 at a second radial distance R, and so on for each different type of sensor dot 1014 in the wells 1004 (with the radial distance for each type being different). Similarly, the rotatable reader plate 1040 is able to comprise at least one of the interrogator optical fibers 1042 at the same radial distances from the point where the axis 1046 intersects the plate 1040.

As a result, the rotation mechanism 1044 is able to selectively rotate the reader plate 1040 about the axis 1046 such that one or more of the interrogator optical fibers 1042 is in optical communication/alignment with one of the distal ends 1010′ thereby enabling the imaging stage to excite and/or receive fluoresced light from the optically coupled sensor dots 1014, and repeat the rotation process until all of the sensor dots 1014/distal ends 1010′ have been interrogated. Further, if both the distal ends 1010′ corresponding to sensor dots 1014 in the same well 1004 and the corresponding interrogator optical fibers 1042 are positioned on the same radius line (i.e. the same straight line from the point where the axis 1046 intersects the wall 1038/plate 1040), then the imaging stage will be able to access all the chemical dots 1014 of each well 1004 simultaneously when the radial line upon which the associated distal ends 1010′ are positioned is aligned with the radial line of the interrogator optical fibers 1042 (by the rotation mechanism 1044). Further, multiple radial lines of the interrogator optical fibers 1042 are able to be used to enable interrogation of a plurality (but not all) of the wells 1004 at the same time. Alternatively, the distal ends 1010′ are able to be positioned in non-radial formations (e.g. a grid or matrix) where the rotation mechanism 1044 is able to be replaced with a translation mechanism that is able to selectively move the reader plate 1040 along the lines of the non-radial formation such that one or more of the interrogator optical fibers 1042 are in optical communication/alignment with one of the distal ends 1010′ thereby enabling the imaging stage to excite and/or receive fluoresced light from the optically coupled sensor dots 1014, and repeat the translation process until all of the sensor dots 1014/distal ends 1010′ have been interrogated. Alternatively, the interrogating components are able to be stationary and the wells/slides 22, 24 are able to move to facilitate the optical alignment.

Thus, in each embodiment, the interrogator 1036 provides the benefit of not having to have each light guide 1010 be coupled with the imaging stage at all times, but rather only needing a subset of interrogator optical fibers 1042 to be used and selectively optically coupled with a desired subset of the sensor dots 1014/wells 1004. This enables both a single light source to be used to excite multiple sensors 1014 and a single reader and/or interrogator 1042 to receive fluoresced signals from multiple sensors 1014.

In some embodiments, the imaging stage is able to both interrogate the chemical sensors and to capture images of the samples (e.g. through a bottom wall of the wells 1004) at the same time. In some embodiments, the imaging stage is able to move from well to well, taking both chemical measurements and image captures at each well 1004 and then time multiplexing the data between different well via hardware. An epimicroscope is able to be used to excite the chemical sensor dots 1014 (e.g. fluorescent sensors) and collect the emitted light/signals from them. In some embodiments, this is done either in the same objective that is doing the optical microscopy (e.g. if the field of view is large enough to cover the dots 1014 and the cells/sample) or it can be done in a parallel one. There are objectives available that can cover the field of view that would be required to cover the whole well. An example of this would be histology microscopes that are compatible with brightfield as well as fluorescent imaging. A telecentric lens would allow for imaging more than one well at a time with acceptable resolution. A high pixel cameras (e.g. 60 megapixel camera) would allow simultaneous imaging of the well (including fluorescent sensors) with submicron resolution. Bioluminescence is able to be collected by having a cooled camera and/or high numerical aperture lens.

Alternatively, the system is able to utilize n pieces of hardware that do the same thing as the time multiplexed single imaging stage by increasing the number of the hardware to have n simultaneous measurements. In some embodiments, one imaging unit is able to have one interrogation for the optical channel, and these are separated from one another by a number of wells. The advantage of this is that optical chemical measurements are able to not be combined to avoid optical cross-talk. Alternatively, the imaging is able to be decoupled from the chemical sensing. For example, having 2*n sensors coming for n wells (e.g. one for oxygen, one for pH), wherein the sensors are able to be connected to a multiplexer to be interrogated. This method would require a long distance lens such as: an epimicroscope above or below for imaging and fibers below or above respectively for exciting and collecting the sensors; wells with molded optics that the sensors/light guides are attached to (These could be used for either sensing from the side, top or bottom of wells). The molded optics could also be a separate piece from the well and attached during manufacturing. In some embodiments, a whole well or multiple wells at once are able to be imaged (e.g. using a large field of view lens and a higher resolution camera and/or multiple lenses and camera combinations in parallel).

In a moving plate system the stage acceleration and hence velocity is limited by liquid sloshing in the wells. If it accelerates to the point that the meniscus moves the imaging will be affected. In contrast, in a moving microscope system there is no intrinsic limit to the acceleration. In some embodiments, the optical body is able to be immersed into the well so that there is no meniscus. This allows for limitless acceleration and higher sensing resolution due to decreased well volume for the same number of cells. If there is no airgap the fluorescent sensing spots/chemical dots are able to be on the bottom of the lid. In some embodiments, the z dimension of each well is as small as possible and the sensor(s) are on the top or bottom of the well. In some embodiments, there can be a tube that allows for pressure relief and culture media replenishment.

FIG. 10Q illustrates a method of implementing a high throughput polybiosensing-imaging system 1000 according to some embodiments. As shown in FIG. 10Q, a slide 1002 having a plurality of wells 1004 is provided at the step 1001. One or more of the wells 1004 are filled with a target sample suspended in a culture media at the step 1003. A lid 1006 including one or more sensors 1012 is positioned on top of the slide 1002 such that the sensors 1012 protrude into a non-central portion of the wells 1004 at the step 1005. In some embodiments, the method further comprises one or more sensor dots 1014 being printed on the wells 1004, coupled to a tip of the sensors 1012, or printed on an insert 1016 that is inserted into the wells 1004. In some embodiments, the insert 1016 has a central aperture 1018.

One or more support structures 1008, 1008′ are positioned on top of the lid 1006 such that light guides 1010 are aligned with the sensors 1012 of each of the wells 1004 at the step 1007. In some embodiments, alignment protrusions 1018 are able to be positioned on top of each of the sensors 1012. In some embodiments, the protrusions 1018 are coupled to and/or integrated into the top of the lid 1006. Alternatively, the protrusions 1018 are able to be coupled to the top of the sensors 1012 (and positioned flush with the top of the lid 1006 when the sensors 1012 are positioned fully within the apertures of the lid 1006). In some embodiments, the bottoms of the support structures 1008, 1008′ are able to have recesses 1024 that surround the bottom of each of the light guides 1010. In some embodiments, positioning the support structures 1008, 1008′ on top of the lid 1006 further comprises moving the protrusions 1018 into the recesses 1024 such that the bottom of the light guides 1010 fully slide into the channels 1020 and abut/optically align with the top of the sensors 1012.

In some embodiments, the lid 1006 is able to have one or more lid protrusions 1030 that extend downward from the lid 1006 into the well 1004. In some embodiments, positioning the lid 1006 on the slide 1002/wells 1004 further comprises extending the lid protrusions 1030 downward into the wells 1004. In some embodiments, a mirror (or other light reflective structure) 1026 is able to be positioned within the support structures 1008, 1008′ to reflect and thereby transmit light between two sections (e.g. vertical and horizontal sections) of the light guides 1010′. Alternatively, the mirrors 1026 are able to be replace or supplemented with one or more lenses that transmit or refract the light between the two sections of the light guides 1010′. In some embodiments, the support structures 1008, 1008′ are able to be transparent to light in between the two sections of the light guides 1010′ and the mirrors 1026. Alternatively, the support structures 1008, 1008′ are able to have a cavity between the two sections of the light guides 1010′ and the mirrors 1026 to enable the passage of light. In some embodiments, the vertical section of the light guides 1010′ are able to be omitted and the support structures 1008, 1008′ are able to be transparent to light or have cavity in between the horizontal sections of the light guides 1010′, the mirrors 1026 and the top of the sensors 1012. In some embodiments, the bottom and/or side walls of one or more wells 1004 of the slides 1002 are able to have one or more well light guides structures 1028.

### Analyte Measurement at Controlled Concentrations

FIG. 11 illustrates a diagram of an implementation to accurately measure analyte consumption at controlled concentrations according to some embodiments.

An analyte can be any chemistry of interest that can be measured and controlled. An example for the analyte of oxygen is described herein but the method is not limited to oxygen.

The present system measures oxygen concentration in media of an enclosed well. The looser the lid (or more oxygen that is permitted to diffuse in through or by other means), the smaller the change in measured concentration will be for a given consumption of oxygen by the cells in the well.

Conversely, the lower the diffusivity of the oxygen barrier, the larger the measured change for a given amount of consumption. If the change is large, however, it risks starving the cells for oxygen. This will either change their metabolism or in extreme cases, kill the cells.

A well 1100 that is well-sealed against oxygen permeation is used. The well 1100 includes a fluorescent sensor 1102 for measuring analyte concentration. Oxygen is provided using a metering device 1104 (e.g., a pump). Feedback is used from measured oxygen level by the analyte measuring device 1106 (e.g., fiber-based) to control the metering device 1104 and keep measured oxygen level constant. The metering device 1104 is signaled to indicate rate of oxygen consumption. The analyte concentration and target analyte concentration are able to be utilized by a gain block 1108 to affect the analyte metering control signal.

The implementation described herein has many advantages. Cells are cultured at the desired level of oxygen. Cells can be cultured at different levels of oxygen to measure effects on metabolism (e.g., shifts to other metabolic pathways). It can thus be determined at what oxygen concentration these metabolic shifts occur, if they are reversible, and more. Cells can be cultured at higher or lower oxygen levels than ambient (e.g., hypoxia experiments for cancer research). In many experimental cases, the oxygen sensor will be operated at a constant oxygen concentration. Linearity is thus no longer an issue. Undesired coupling between cell growth and oxygen level in media is eliminated. Step response becomes possible. Cross coupling of different sensors becomes negligible if analytes are kept constant. The approach can be used for any media property that can be measured and controlled. Examples include: glucose, CO2, pH (acid or base generation by cells) and others.

### Software and Analytics

The external electronic device 8 is able to comprise an analytics engine that is able to pre-process raw timed data received from the sensors (e.g. O2, pH, glucose, impedance, etc.). For example, the engine is able to pre-process the data from raw data to decimated data to raw unified data to calibrated unified data to filtered unified data to normalized unified data. Further, the engine is able to process the image data into image features data and the normalized unified data into model ready data. For example, the normalizing is able to comprise normalizing based on cell number, size, growth stage, etc. (e.g. using a number of cells from imaging to normalize changes in O2 and/or pH per cell; using fluorescent markers of different states of cells and quantifying the fluorescent signal of interest to normalize another measurement; using environmental sensor data (e.g. CO2 levels) to determine when the incubator door was open and removing the associated tainted data). Finally, the engine is able to perform modeling and analysis on the data to derive insights using pairwise correlations (such as correlation matrices per Genius well™), data clustering, regression analysis, etc. The correlation matrices are able to provide measured maps for polymodal cellular signals per Genius wells™. This leads to more informed decision-making because maps are functions of intrinsic and/or extrinsic heterogeneity caused by the different genetics, metabolomics, and environments of the samples over time and space. For example, in some embodiments the analytics engine is able to generate a cell health index that indicates a health value or metric of the cell or cells within one or more of the wells 24.

Data signals corresponding to sensed measurements from each of the different sensor types are transmitted to the electronic device(s) 8, such as a computer, and can be compiled within a unified common user interface that enables operation of the for different measurement modes under one software interface. As previously described, multiple different sensing modes can be implemented and executed within each well, where the different sensing can be performed by different sensor types. Example sensor types include, but are not limited to, impedance sensors, temperature sensors, pH sensors, O2 sensors, lactose, lactate, selective ion, glucose sensors, and secretomes. These sensors are able to be light based, electrical based and/or electrochemical based. The data sensed by each sensor is transmitted by corresponding transmitters (e.g. optical fibers, circuitry, networks) to corresponding measurement devices, such as measurement meters, the results of which are transmitted to an electronic device for analysis. Data analytics is performed by the electronic device for data fusion and polymodal analysis using the received measured data. Data analytics algorithms enable data annotation, data normalization to prepare model ready data, data viewing, data integration and querying new information. In some embodiments, as described above, well/sample sensor data is able to be corrected using incubator 2 environment sensor data. Specifically, as described above, the incubator 2 is able to comprise an environmental sensor board or other sensor apparatus that is able to detect a temperature, pressure, humidity, O2 and/or CO2 concentration within the incubator 2 (e.g. around the wells/slide). In particular, the temperature values are able to be used to correct sensed pH, lactate, glucose, O2 or other temperature dependent values detected within the media/wells 24. Similarly, the atmospheric pressure and relative humidity values within the incubator 2 are able to be used to adjust O2 levels detected within the media/wells 24. In some embodiments, the system 10 is able to determine whether the incubator 2 door was opened (such that values during that time may be inaccurate) based on measured relative humidity and CO2 levels. As a result, these corrected values are able to be used in the by the analytics engine to produce more accurate processed data and models and thus determine more accurate and powerful insights about the samples/data.

Thus, the system 10 provides the benefit of providing a data analytics engine stored on the devices 8 that, along with polysensing data capturing capabilities, enables combining data from several sources in order to form a unified picture. Specifically, the engine connects multi dimensional datasets, queries relationships, and processes orthogonal features to create distinct, otherwise unattainable insights enabling a new comprehensive view of cellular behavior. Indeed, by polysensing (i.e. sensing multiple different chemistries/properties of the same sample of the same well at the same time) the same live cells, the system 10 is able to generate connected datasets without perturbing the environment of the sample. This leads to lower noise in the data because of the ability to make internal validations of readouts from multiple angles and new correlations that can be complementary, orthogonal or synergic. For example, sensors that measure the same attribute from different wells at the same time help to normalize out external influences for more accurate statistical analysis and hypothesis testing, wherein lack of expected correlation can be used to discover hardware or experimental errors. As another example, sensors that measure independent (complementary) types of information at the same time are able to indicate correlation between the values/measurements due to biological effects being measured (e.g. normal vs. malignant cell behavior). Finally, using optical or impedance data to count cells and normalize metabolic measurements provides data that is normalized per well such that it is more repeatable between experiments. Thus, in sum, the simultaneous measurements produced by the described polysensing not only improves the quality of each sensing modality, but can be used to normalize the readings of each other and internally calibrate one modality against another one. Moreover, the imaging can be used to track the cell cycle and match over time with changes in metabolites and the fluorescent labeling of proteins is able to be used to track changes and compare correlations.

The present application has been described in terms of specific embodiments incorporating details to facilitate the understanding of the principles of construction and operation of the polybiosensing-imaging system. Many of the components shown and described in the various figures can be interchanged to achieve the results necessary, and this description should be read to encompass such interchange as well. As such, references herein to specific embodiments and details thereof are not intended to limit the scope of the claims appended hereto. It will be apparent to those skilled in the art that modifications can be made to the embodiments chosen for illustration without departing from the spirit and scope of the application. For example, the light guides, wave guides and/or optical fibers described herein are able to be replaced and/or supplemented with lenses and/or mirrors that direct the light in the same manner as the optical fibers. Further, it is noted that although the light sources are generally described herein as light emitting diodes, other types of light sources or combinations thereof are able to be used.

Although the sensing capabilities described above are generally directed to chemical dot sensors (sensing mechanisms) positioned in and/or around the well, it is understood that the polybiosensing-imaging system can be configured to include any combination of a variety of different sensor types including, but not limited to, electrically conductive sensors (e.g. for measuring impedance, pH, glucose, O2, lactate and/or other chemistries), immersion probes, chemically sensitive films/foils/dots, pass-through sensors, or a hybrid system. As described herein, the indicators, foil or chemical sensitive dots are able to comprise analyte-sensitive optically signaling material (e.g. optical fluorescence). However, sensing modes are not restricted to optical fluorescence and are able to include one or more of enzymatic, amperometric, solid-state and/or electrochemical sensors. In some embodiments, these sensors are implemented as immersion probes (e.g. sensor 710).

In regard to immersion probes, a probe-configuration sensor (long thin >syringe/tube/needle=) can be chosen for each measurement mode. Cables/wires are connected, as appropriate, to base stations/readers/data loggers. The immersion probes (e.g. 710) are selected so that each, including any larger heads/bases, can be physically put into the well at the same time. Well lids are configured to cover the rest of the well during extended measurement time periods. In regard to chemically sensitive films/foils/dots, consumable sensor films/foils or semi-durable sensor dots can be attached to well bottom or sides as sensors. Films/foils/dots are selected so that they fit on select areas of well bottom and/or sides, and/or film/foil can be cut to fit to target areas on well bottom and/or sides, or placed in separate wells in case of high cross-talk in wells of smaller sizes. In this case, polysensing and imaging is done per plate (that holds multiple wells). In some embodiments, the system 10 is able to measure pH, O2 and/or glucose using printed circuits and/or sensors that have been biochemically or enzymatically functionalized for detecting the analyte. In the case of each of the types of sensors described herein, the sensors are able to be constructed/positioned on the side walls and/or bottom of each well, within the lid, and/or within/on an insert that is positioned within the wells and/or connected to the lid.

The terms biosensing-imaging system or multi-sensing imaging, multi-modal sensing and imaging, polybiosensing and bioimaging, 2D or 3D or 4D polysensing and imaging, and polysensing and imaging, are used interchangeably in this document.

1. A hybrid polybiosensing-imaging system comprising:
a sensor positioning apparatus comprising:
a base having a well recess and a plurality of imaging holes within the well recess; and
a slide holder having a central slide window and one or more optical passages that extend from the central slide window to a perimeter of the slide holder opposite the central slide window; and
a well slide including a plurality of wells, the well slide positioned within the well recess such that each of the wells is above one of the imaging holes;
wherein the slide holder is able to pivot with respect to the base between a closed position where the slide holder is substantially parallel to the base and the well slide is within the central slide window and an open position where the slide holder is angled away from the base.

2. The system of claim 1, further comprising a lid covering each of the wells and including a separate sensor hole and syringe hole corresponding to each of the wells.

3. The system of claim 2, wherein the lid further comprises a separate sensor support tab corresponding to each of the wells, wherein for each of the wells, the sensor support tab protrudes upward from the lid adjacent to a top opening of the sensor hole corresponding to the well.

4. The system of claim 2, wherein the lid further comprises a separate rim guide corresponding to each of the wells, wherein for each of the wells, the rim guide protrudes downward from a bottom of the lid such that the rim guide abuts a top rim of the well.

5. The system of claim 2, wherein for each of the wells, the sensor hole corresponding to the well is positioned adjacent an outer wall of the well and the syringe hole corresponding to the well is positioned adjacent to an inner wall of the well opposite the outer wall.

6. The system of claim 5, wherein for each of the wells, a central axis of the syringe hole corresponding to the well is angled such that the central axis points from a top right corner of the inner wall of the well to a bottom left corner of the inner wall of the well.

7. The system of claim 1, wherein the optical passages become narrower such that an outer opening of each of the optical passages facing the perimeter of the slide holder is larger than an inner opening of each of the optical passages facing the central slide window.

8. The system of claim 7, further comprising one or more optical probes that each fit within the outer opening of one of the optical passages and having one or more optical fibers that fit within the inner opening of the one of the optical passages.

9. The system of claim 1, further comprising one or more guide plates coupled to the slide holder such that the guide plates extend over the central slide window, wherein when the slide holder is pivoted from the open position to the closed position, the guide plates contact angled outside-facing walls of the wells of the well slide causing the well slide to align with a center of the central slide window.

10. The system of claim 2, further comprising a flexible forked circuit including a network interface at a base of the circuit, one or more first electrical couplers at a first finger of the forked circuit and one or more second electrical couplers at a second finger of the forked circuit, wherein the first and second electrical couplers are configured to electrically couple the network interface with one or more sensors positioned within the sensor holes of the lid.

11. The system of claim 1, further comprising an electrical interconnect substrate including a plurality of electrodes positioned under the wells of the slide and an impedance printed circuit board configured to electrically couple with the electrodes when the slide holder is in the closed position.

12. The system of claim 11, wherein the impedance printed circuit board is positioned on a printed circuit board platform at an end of the slide holder and has one or more electrical coupling pins that protrude through an impedance window of the end of the slide holder to electrically couple with the electrodes when the slide holder is in the closed position.

13-60. (canceled)
